(19) United States
(12) Patent Application Publication
(10) Pub. No.: US 2003/0219839 A1
(43) Pub. Date: Nov. 27, 2003

(54) Anti-PDGF antibodies and methods for producing engineered antibodies

(76) Inventors: Katherine Bowdish, Del Mark, CA (US); Shana Frederickson, Solana Beach, CA (US); Anke Kretz-Rommel, San Diego, CA (US)

Correspondence Address: Mark Farber, Esq., c/o Alexion Pharmaceuticals, Inc., 352 Knotter Drive, Cheshire, CT, 06410, US

(21) Appl. No.: 10/251,215

(22) Filed: Sep. 20, 2002

Related U.S. Application Data
(60) Provisional application No. 60/323,537, filed on Sep. 20, 2001.
(60) Provisional application No. 60/323,544, filed on Sep. 20, 2001.
(60) Provisional application No. 60/379,980, filed on May 13, 2002.

(51) Int. Cl. G01N 33/53; G01N 33/542; C07H 21/04; C12P 21/02; C12N 5/06; C07K 16/00

(52) U.S. Cl. 435/7.9; 435/69.1; 435/320.1; 435/326; 530/387.1; 536/23.53

(57) ABSTRACT

Methods of making and selecting engineered antibodies and/or antibody fragments provide maximized binding affinity for a predetermined target and minimized immunogenicity when such antibodies are administered to a target species. Libraries containing variants of the engineered antibodies are also provided. In particularly useful embodiments, anti-PGDF antibodies and compositions are produced which are useful in the treatment of various cancers.

### RELATED APPLICATIONS

This application claims priority to U.S. Provisional Application No. 60/323,537 filed on Sep. 20, 2001; U.S. Provisional Application No. 60/323,544 filed on Sep. 20, 2001; and U.S. Provisional Application No. 60/379,994 filed on May 13, 2002.

### BACKGROUND

1. Technical Field

The present description relates to antibodies and, more specifically, to engineered antibodies and antibody fragments derived from one species which preferentially bind a target object and which have reduced immunogenicity in a different species. More particularly, anti-PDGF antibodies and engineered anti-PDGF antibodies are provided.

2. Background of Related Art

Platelet-derived growth factor (PDGF) was originally discovered as a platelet α-granule release product, but subsequently a number of different PDGF-secreting cell types have been identified. PDGF is a homo- or heterodimeric protein composed of at least two of four homologous chains: A, B, C and D, joined by disulfide bonds. The PDGF signal is mediated via 2 high affinity receptors termed PDGFR-α and -β. Whereas PDGF-BB and -AB can signal through both receptors, PDGF-M and PDGF-CC can only bind to the α-receptor, PGDF-DD binds and signals exclusively through the β receptor. PDGF acts as a potent mitogen, chemoattractant and survival factor for mesenchymal cells. It plays a critical role in physiological repair mechanism, but is also involved in the pathogenesis of various proliferative diseases. The finding that the v-sis oncogene of simian sarcoma virus is a retroviral homolog of the PDGF B-chain gene (Doolittle, et al. Science Jul. 15, 1983; 221(4607): 275-277, Deuel, et al. Science Sep. 30, 1983; 221 (4618): 1348-1350) suggested a role of PDGF in oncogenesis.

Initial evidence for the involvement of PDGF in brain tumor development resulted from the observation that intracerebral injection of simian sarcoma virus carrying v-sis can induce brain tumor formation in monkeys Deinhardt, et al. (ed) Viral Oncology 357-398, New York, Raven Press, 1980. Only cell lines that express PDGF receptors could be transformed in an autocrine fashion by transfection with v-sis. Expression of PDGF BB and PDGF DD and their receptors have been demonstrated in human neuroblastoma, neuroepithelioma, meningiomas, Ewing's sarcoma, astrocytoma, glioblastoma, Kaposi's sarcoma, mesothelioma and mesothelioma cell lines, choriocarcinoma, pancreatic carcinoma, gastric carcinoma, osteosarcoma, esophageal cancer, fibrosarcoma, malignant epithelial cells in primary human lung carcinoma, leiomyosarcoma, liposarcoma, paraganglioma, angiosarcoma, hemangiopericytoma, sarcoma NOS, synovial sarcoma, chondrosarcoma, and uterine stromal sarcoma, mammary carcinoma, colorectal cancer, small-cell lung carcinoma, non-small cell lung cancer, malignant fibrous histiocytoma, smooth muscle cell tumor, and prostrate cancer, suggesting the existence of functional autocrine and paracrine loops.

A number of studies involving brain tumor cells have been performed using inhibitors of PDGF. For example, Chin et al, Clin Cancer Res May 1997; 3(5): 771-776, used the inhibitor K252a together with Glioma cell lines U87 and T98G. K252a treatment blocks receptor autophosphorylation in response to PDGF, and leads to reduced proliferation in the cell lines. Moreover, apoptosis is induced in U87 cells.

Johnson et al, Nature Oct. 3, 1985; 317 (6036): 438-440 used antibodies against PDGF to inhibit acute transformation by simian sarcoma virus. Fibroblasts were transformed by SSV/SSAV such that foci could be detected after a few days. Anti-PDGF antibody suppresses focus formation early after infection (8-12 days). However at 20 days, there was no difference in the number of foci between anti-PDGF and control antibodies.

Todo et al., (1996) J. Neurosurg 84(5):852-8 used anti-PDGF antisera to inhibit conditioned-medium stimulated DNA synthesis in meningioma cells, suggesting a role for PDGF-B related molecules in meningioma cell proliferation.

Two PDGF antagonists, trapidil and suramin, have been shown to inhibit growth-factor-induced glioma and meningioma cell proliferation. Suramin is a nonspecific purinergic receptor antagonist that inhibits EGF, IFG-1 and PDGF-BB induced proliferation of meningioma cells by up to 40-70% (Schrell et al., Neurosurg Apr. 1995; 82(4): 600-607). Trapidil has been shown to preferentially inhibit PDGF-positive than PDGF-negative cell lines. However, both antagonists are not specific for PDGF, but also act on other tyrosine kinase receptors. Other types of receptor antagonists are selective PDGF-receptor phosphorylation blockers and dominant negative receptors. These treatments have the disadvantage that they also could effect normal brain tissue since the receptor is present in normal as well as malignant brain cells.

Antibodies are proteins produced by lymphocytes known as B cells in vertebrates in response to stimulation by antigens. The basic structural unit of an antibody (a.k.a. immunoglobulin (Ig)) molecule consists of four polypeptide chains which come together in the shape of a capital letter “Y”. Two of the four chains are identical light (L) chains and two are identical heavy (H) chains. There are five different kinds (isotypes) of heavy chains which divide antibodies into five classes, namely, IgA, IgD, IgE, IgG and IgM. In addition, there are two different isotypes of light chains designated K and A. Each class of heavy chains can combine with either of the light chains. The heavy and light chains each contain a variable region (VH and VL, respectively) that is involved in antigen binding and a constant (C) region. The antigen binding site is composed of six hypervariable regions (a.k.a. complementarity determining regions (CDRs)). Three CDRs from the heavy chain and three CDRs from the light chain are respectively positioned between four relatively conserved anti-parallel 8-sheets which are called framework regions (FR1, FR2, FR3 and FR4), on each chain. By convention, numbering systems have been utilized to designate the location of the component parts of VH and VL chains. The Kabat definition is based on sequence variability and the Chothia definition is based on the location of structural loop regions.

Antibodies have become an object of intense research and development in the continuing quest for therapeutic treatment of various diseases and conditions. This is due to the natural ability of antibodies to seek out and bind to specific targets in vivo. The ability to manipulate antibodies took a great leap forward when Kohler and Milstein (Kohler et al., Nature 256:495-497 (1976)) developed the hybridoma technique of producing monoclonal antibodies (“mAb”) based on mouse antibodies. This generally involves immunizing a mouse with an antigen, fusing spleen cells from the immunized mouse with myeloma cells to create a hybridoma. Individual cells are selected from the hybridoma which secrete a single or so-called “monoclonal antibody” specific for the target antigen. It was thought that monoclonal antibodies could be raised against a variety of targets and then administered to humans to achieve considerable therapeutic or diagnostic effect. Unfortunately, in many instances, monoclonal antibodies are recognized by the human immune system as being a foreign substance not ordinarily occurring in the human body. This is referred to as immunogenicity or antigenicity in humans. For this reason, when antibodies of non-human origin are administered to humans, anti-non-human antibody antibodies are generated which result in enhanced clearance of the non-human antibodies from the body, thus reducing or completely blocking their therapeutic or diagnostic effects. Hypersensitivity reactions may also occur. As a result, much research has been directed to manipulating the sequence and structure of antibodies to make them more human-like and therefore avoid immunogenicity in humans leading to the development of genetic engineering technologies known as “humanization.”

The first humanization strategies were based on the knowledge that heavy and light chain variable domains are responsible for binding to antigen, and the constant domains for effector function. Chimaeric antibodies were created, for example, by transplanting the variable domains of a rodent mAb to the constant domains of human antibodies (e.g. Neuberger M S, et al., Nature 314, 268-70, 1985 and Takeda, et al., Nature 314, 452-4,1985). Although these chimaeric antibodies induce better effector functions in humans and exhibit reduced immunogenicity, the rodent variable region still poses the risk of inducing an immune response. When it was recognized that the variable domains consist of a beta sheet framework surmounted by antigen-binding loops (complementarity determining regions or CDR's), humanized antibodies were designed to contain the rodent CDR's grafted onto a human framework. Several different antigen-binding sites were successfully transferred to the same human framework, often using the human framework with the closest homology to the rodent sequence (e.g. Jones P T, et al., Nature 321, 522-5, 1986; Riechmann L. et al., Nature 332, 323-327, 1988; and Sato K. et al., Mol. Immunol. 31, 371-8, 1994). Alternatively, consensus human frameworks were built based on several human heavy chains, (e.g., Carter P. et al., Proc. Nat. Acad. Sci. USA 89, 487-99, 1992). However, simple CDR grafting often resulted in loss of antigen affinity. Other possible interactions between the β-sheet framework and the loops had to be considered to recreate the antigen binding site (Chothia C, et al., Mol. Biol. 196, 901-917,1987).

Comparison of the essential framework residues required in humanization of several antibodies, as well as computer modeling based on antibody crystal structures revealed a set of framework residues termed as “Vernier zone residues” (Foote J., et al., Mol Biol 224, 487-99, 1992) that most likely contributes to the integrity of the binding site. In addition, several residues in the VH-VL interface zone might be important in maintaining affinity for the antigen (Santos A D, et al., Prog. Nucleic Acid Res Mol Biol 60, 169-94 1998). Initially, framework residues were stepwise mutated back to the rodent sequence (Kettleborough C A, et al. Protein Engin. 4, 773-783, 1991). However, this mutation approach is very time-consuming and cannot cover every important residue.

For any particular antibody a small set of changes may suffice to optimize binding, yet it is difficult to select from the set of Vernier and VHNL residues. Combinatorial library approaches combined with selection technologies (such as phage display) revolutionized humanization technologies by creating a library of humanized molecules that represents alternatives between rodent and human sequence in all important framework residues and allows for simultaneous determination of binding activity of all humanized forms (e.g. Rosok M J, J Biol Chem, 271, 22611-8,1996 and Baca M, et al. J Biol Chem 272, 10678-84, 1997).

The above approaches graft each of the 6 rodent CDR's into a human framework thereby introducing a considerable number of potentially problematic rodent residues. Accordingly, it would be advantageous to provide engineered antibodies based on antibodies from an originating species which exhibit reduced immunogenicity while maintaining an optimum binding profile that can be administered to a target species for therapeutic and diagnostic purposes. It would also be advantageous to provide engineered PDGF antibodies which exhibit reduced immunogenicity while maintaining an optimum binding profile that can be administered to a target species for therapeutic and diagnostic purposes.

### SUMMARY

Highly selective, therapeutically useful anti-PDGF-BB antibodies acting as PDGF antagonists are provided. A high affinity PDGF-BB antibody derived from murine spleen and bone marrow has been isolated by phage display. The antibody recognizes human, but not rat PDGF-BB. Also provided is a humanized anti-PGDF-BB antibody, generated by selecting either murine or human CDRs and altering certain framework amino acids in the human framework most closely related to the mouse framework. Also provided is a humanized anti-PGDF-BB antibody, generated by selecting either rabbit or human CDRs and altering certain framework amino acids in the rabbit framework. The mouse antibody (C1), rabbit antibody (F3), as well as the humanized antibodies (E1 and B1) compete effectively with the PDGFβ receptor for PDGF-BB. The mouse C1 inhibits proliferation of the neuroblastoma cell lines HTB11 (IMR 32) and CCL127 (NB41A3), the neuroepithelioma cell line HTB10 (SKNMC), Ewing's sarcoma cell line HTB166 (RDES), astrocytoma cell line U87, glioblastoma cell line T98G, human lung carcinoma cell lines NCIH1651, NCI1876, NCIH2228, and lung adenocarcinoma cell line NCIH23 indicating that the antibody is therapeutically useful for the treatment of various cancers.

Also provided is an antibody light chain including at least one CDR derived from a CDR selected from the group consisting of CDR1, CDR2 and CDR3 of antibody C1 as set forth in FIG. 14a.

Moreover, an antibody heavy chain is provided which includes at least one CDR derived from a CDR selected from the group consisting of CDR1, CDR2 and CDR3 of antibody C1 as set forth in FIG. 14b.

Optionally, two or more CDRs are selected from the said groups; in one embodiment, all three CDRs may be selected from said groups. The CDRs may be modified by amino acid substitution, addition or deletion according to methods known in the art for antibody engineering and discussed in more detail herein.

Preferred heavy and light chains are set forth in FIGS. 14a and 14b respectively.

Framework regions (FRs) are preferably derived from or identical to the FRs set forth in FIGS. 15a and 15b in respect of humanized antibodies B1 and E1.

Light and heavy chains may be combined to form entire antibodies. Antibodies may be full length immunoglobulins, comprising constant domains, or antigen-binding immunoglobulin fragments, including Fab, F(ab′)₂, Fv and scFv. Suitable antibodies are antibodies B1 and E1, as set forth in FIGS. 15a and 15b.

In another embodiment, there is provided a method for treating tumors selected from the group consisting of neuroblastoma, neuroepithelioma, meningiomas, Ewing's sarcoma, astrocytoma, glioblastoma, Kaposi's sarcoma, mesothelioma and mesothelioma cell lines, choriocarcinoma, pancreatic carcinoma, gastric carcinoma, osteosarcoma, esophageal cancer, fibrosarcoma, malignant epithelial cells in primary human lung carcinoma, leiomyosarcoma, liposarcoma, paraganglioma, angiosarcoma, hemangiopericytoma, sarcoma NOS, synovial sarcoma, chondrosarcoma, and uterine stromal sarcoma, mammary carcinoma, colorectal cancer, small-cell lung carcinoma, non-small cell lung cancer, malignant fibrous histiocytoma, smooth muscle cell tumor, prostrate cancer the method including comprising administering to a subject in need thereof an effective amount of an anti-PDGF antibody. The antibody is preferably an anti-PDGF BB antibody. The antibody may be used in combination therapy with other chemotherapeutic agents in the treatment of various cancers.

In another aspect, a method is described herein for providing an optimized engineered antibody or antibody fragment includes providing a first antibody having specificity for a target; determining the sequence of at least a portion of the first antibody; comparing the sequence of at least a portion of the first antibody to a reference library of antibody sequences or antibody fragment sequences from a target species; selecting at least one sequence from the library which demonstrates a high degree of homology to the at least a portion of the first antibody and which contains a CDR3 region; obtaining the CDR3 region from the first antibody; and incorporating the CDR3 region from the first antibody into the position previously occupied by the CDR3 region of the at least one sequence from the library to form an optimized engineered antibody or antibody fragment. The sequences referred to above may be amino acid sequences or nucleic acid sequences. The antibody fragment referred to may be selected from the group consisting of scFv, Fab, F(ab′)₂, Fd, diabodies, antibody light chains and antibody heavy chains. The target species referred to above may be human. The sequence of at least a portion of the first antibody referred to above may be a variable region sequence and the antibody fragment sequences from a target species may be variable regions.

In addition, a variable region of the first antibody consisting of FR1, CDR1, FR2, CDR2 and FR3 may be aligned with germline or rearranged sequences of the target species to determine degree of homology. In a further aspect, the method of the above paragraph also includes determining non-homologous positions between the at least a portion of the first antibody and the at least one sequence from the library which demonstrates a high degree of homology and constructing variants of optimized engineered antibodies or antibody fragments which contain either the original first antibody residue at a non-homologous position or the original library sequence residue at the same non-homologous position. In addition, a phagemid or phage library may be generated which displays the variants of optimized engineered antibodies or antibody fragments. Moreover, the phagemid or phage library may be panned for activity against the target object and the phage or phagemid particles which preferentially bind to the target object are isolated.

Moreover, the CDR1 region from the first antibody may optionally be obtained and incorporated into the position previously occupied by the CDR1 region of the at least a portion of the at least one sequence from the library. Similarly, the CDR2 region from the first antibody may optionally be obtained and incorporated into the position previously occupied by the CDR2 region of the at least a portion of the at least one sequence from the library. In addition, a choice between either the CDR1 region or the CDR2 region or both of the first antibody and those of the target species may be made. The reference library referred to above may contain human rearranged antibody sequences. In one aspect, the target object is PDGF.

Also provided is a library of engineered antibodies or antibody fragments for selecting optimized antibodies or antibody fragments, the library including variant engineered antibodies or fragments, the variants having framework regions derived from an antibody native to a target species, the framework regions exhibiting a high degree of homology to the framework region of a first antibody having specificity for a predetermined target, a CDR3 region derived from the first antibody having specificity for a predetermined target, a combination of CDR1 and CDR2 regions from either the first antibody or the antibody native to the target species, and a combination of amino acids in the VHNL interface and/or Vernier zone, the combination of amino acids representing a choice between those present in the antibody native to the target species and those present in the first antibody.

In yet another aspect, a humanized composite antibody or functional fragment of a humanized composite antibody in accordance with this disclosure includes framework regions from one or more human antibody sequences and CDR regions from two different non-human sources. In another aspect, a humanized composite antibody or functional fragment of a humanized composite antibody in accordance with this disclosure contains framework regions from both germline and re-arranged human antibody sequences and CDR regions from two different sources, at least one of which is non-human. In yet another embodiment, a humanized composite antibody or functional fragment of a humanized composite antibody in accordance with this disclosure includes framework regions from one or more human antibody sequences, a non-human CDR3 and at least one of CDR1 or CDR2 derived from a consensus of non-human antibody sequences.

Selecting the components for the humanized composite antibodies is achieved by a method that includes the steps of: (i) establishing an antibody consensus sequence for a plurality of members from a panel of non-human antibodies that bind to a target; (ii) substituting a CDR3 from an individual member of the panel of non-human antibodies for the CDR3 of the consensus sequence to form a composite sequence; and (iii) comparing the composite sequence to a database of human antibody sequences and selecting at least one human antibody sequence based on homology to the composite sequence.

The human antibody sequence(s) selected in this manner provide the framework regions for the humanized composite antibodies. At least the CDR3 for the humanized composite antibodies or functional fragment of a humanized composite antibody is selected from the panel of non-human antibodies that bind to an antigen. The CDR1 and CDR2 for the humanized composite antibodies or functional fragment of a humanized composite antibody can be selected from the panel of non-human antibodies that bind to an antigen, from the consensus sequence or from the human sequence(s) selected based on homology to the composite sequence. In particularly useful embodiments, the CDR3 used in the humanized composite antibodies or functional fragment of a humanized composite antibody is the CDR3 substituted into the consensus sequence to form the composite sequence and the CDR1 and CDR2 used in the humanized composite antibodies are selected from one or more different members of the panel or are from the consensus sequence.

In one embodiment, the composite sequence is compared to a database of sequences for re-arranged antibodies (such as, for example, the Kabat database) and a single human antibody sequence is selected based on homology to the composite sequence. In this embodiment, framework regions from a single antibody are employed to make the humanized composite antibodies or functional fragment of a humanized composite antibody.

In another embodiment, a portion of the composite sequence is compared to a database of germline sequences (such as, for example, the V-base database), and a second portion of the composite sequence is compared to a database of sequences for re-arranged antibodies (such as, for example, the Kabat database). At least one human sequence is selected from each database based on homology to at least a portion of the composite sequence. In this embodiment, framework regions from two antibody sequences are employed to make the humanized composite antibodies or functional fragment of a humanized composite antibody, with one or more framework regions being selected from germline sequences and one or more framework regions being selected from re-arranged antibody sequences.

A further point of diversity may be introduced with respect to the above method which includes determining non-homologous (such as the Vernier or V_{H}/V_{L} interface) positions between the composite sequence and the selected human sequence(s) and constructing variants of optimized engineered antibodies or antibody fragments which contain either the original composite sequence residue at a non-homologous position or the original residue from the framework of the selected human sequence(s).

The sequences referred to above may be scFv, Fab, F(ab′)₂, Fd, diabodies, antibody light chains and antibody heavy chains.

### BRIEF DESCRIPTION OF THE DRAWINGS

FIG. 1 is a schematic diagram depicting phagemid pRL4.

FIG. 2 is a schematic diagram depicting phagemid pRL5.

FIG. 3a depicts a set of human germline gene sequences having a high degree of homology to rabbit antibody F3 light chain variable sequences. VBASE nucleotide sequence alignment is shown between F3 light chain and human germline sequences. Dashes indicate nucleotides which are identical to F3. Framework positions selected for diversification are underlined.

FIG. 3b depicts a set of human germline gene sequences having a high degree of homology to rabbit antibody F3 heavy chain variable sequences. VBASE nucleotide sequence alignment is shown between F3 heavy chain and human germline sequences. Dashes indicate nucleotides which are identical to F3. Framework positions selected for diversification are underlined.

FIG. 4a depicts oligonucleotides having overlapping regions for assembly of a humanized F3 light chain.

FIG. 4b depicts oligonucleotides having overlapping regions for assembly of a humanized F3 heavy chain.

FIG. 5 depicts Key framework residues diversified in humanization of the F3 antibody. The asterisks identify linked positions (VH:48-49 and VL: 80-83) and indicate a coupled diversification that limits the selection to other human or both rabbit sequences. ¥ indicates amino acid choices that were neither human nor mouse that resulted from degenerate coding used for diversification at that position.

FIGS. 6a-6e depict a schematic of the synthetic assembly of humanized F3 library by PCR using overlapping degenerate nucleotides. FIG. 6a represents assembly of the F3 light chain. FIG. 6b represents assembly of the F3 heavy chain. FIG. 6c represents assembly of the complete humanized F3 library. FIGS. 6d and 6e show the sequences of the degenerate overlapping oligonucleotides. FIGS. 6f and 6g show the assembled degenerate oligonucleotides and corresponding amino acid sequence.

FIG. 7 depicts the results of screening of humanized F3 clones after four rounds of panning in solid phase PDGF ELISA. Clones C11, C12 and A12 were selected for sequencing and binding analysis.

FIG. 8a depicts a schematic comparison of a humanized light chain with rabbit and desired human sequence. Human CDRs are underlined. All CDRs are bold.

FIG. 8b depicts a schematic comparison of a humanized heavy chain with rabbit and desired human sequence. Human CDRs are underlined. All CDRs are bold.

FIG. 9 is a graph depicting the ability of the humanized F3 antibodies to compete with the PDGF-β receptor for the cytokine PDGF-BB was determined in a competition assay. Binding of the humanized anti-PDGF F3 antibody variants (C11, C12, A12) to their target epitope on the PDGF-BB cytokine prevents PDGF-BB binding to the PDGF receptors coated on the plates. All three humanized F3 variants displayed similar binding profiles to the profile of the original non-humanized F3 antibody.

FIG. 10 shows the summary of tritiated thymidine proliferation assays.

FIG. 11 shows the results of angiogenesis assays.

FIG. 12 shows the induction of apoptosis by Anti-PDGF antibody in A172 and T98G cells after 3 days.

FIG. 13 shows the induction of apoptosis in the presence of the pan-caspase inhibitor z-vad.

FIG. 14a shows the sequence of C1 mouse anti-PDGF Fab light chain indicating frameworks and CDRs.

FIG. 14b shows the sequence of C1 mouse anti-PDGF Fab heavy chain indicating frameworks and CDRs.

FIG. 15a illustrates the sequence of humanized clones E1 and B1 indicating human or murine framework positions and CDRs where there is a choice. Overlapping regions of sequence are indicated in similar colors. Mouse residues are shown in purple and human residues are shown in blue.

FIG. 15b illustrates the sequence of humanized clones E1 and B1 indicating human or murine framework positions and CDRs where there is a choice. Overlapping regions of sequence are indicated in similar colors. Mouse residues are shown in purple and human residues are shown in blue.

FIG. 16a illustrates assembly of oligonucleotides for humanization of light chains.

FIG. 16b illustrates assembly of oligonucleotides for humanization of heavy chains.

FIG. 16c is a list of oligonucleotide primers used for assembly of humanized light chain regions which depicts oligonucleotides corresponding to human and murine CDR regions. Oligonucleotides designated with a B or D are human CDRs and the others are mouse CDRs.

FIG. 16d is a list of oligonucleotide primers used for assembly of humanized heavy chain regions with depicts oligonucleotides corresponding to human and murine CDR regions. Oligonucleotides designated with a B are human CDRs and the others are mouse.

FIG. 17 is a graph illustrating reactivity of mouse C1 to various sources of PDGF. Binding of C1 to various forms of PDGF was determined in solid phase ELISA.

FIG. 18 is a graph illustrating the results of competition ELISA. Mouse C1 and rabbit A8 anti-PDGF antibodies compete with PDGF-beta receptor for binding to PDGF-BB.

FIG. 19 is a bar graph illustrating the results of a Luciferase assay. C1 competes with PDGF-beta receptor for binding to PDGF-BB thereby preventing signal induction by PDGF-BB as measured by luciferase activity.

FIG. 20 is a graph illustrating the results of competition ELISA. Mouse C1 and humanized E1 and B1 anti-PDGF antibodies compete with PDGF-beta receptor for binding to PDGF-BB.

FIG. 21 is a graph illustrating the results of a proliferation assay based on LDH using HTB11 cells. The results indicate inhibition of cell proliferation by C1 anti-PDGF antibody, but not by the control 57-38.1 anti-F protein antibody. Experiments were done in duplicate and repeated 3 times. P-values were determined using 2-tailed student's t-test.

FIG. 22 is a graph illustrating the results of a proliferation assay based on LDH using CCL127 cells.

FIG. 23 is a graph illustrating the results of a proliferation assay based on LDH using HTB10 cells.

FIG. 24 is a graph illustrating the results of a proliferation assay based on LDH using HTB166 cells.

FIG. 25 is a graph illustrating the results of a proliferation assay based on LDH using U87 cells.

FIG. 26 is a graph illustrating the results of a proliferation assay based on LDH using A172 cells.

FIG. 27 is a graph illustrating the results of a proliferation assay based on LDH using T98G cells.

FIG. 28 is a graph illustrating the results of a proliferation assay based on LDH using CCL147 murine cells.

### DETAILED DESCRIPTION OF PREFERRED EMBODIMENTS

The techniques described herein provide engineered antibodies, especially optimized engineered antibodies which are highly active against PDGF and which reduce the risk of immunogenicity in humans. It has been surprisingly found that incorporation of a CDR3 region derived from one species in place of the CDR3 region of a variable region from an antibody derived from a target species which has been manipulated in accordance with the present disclosure is sufficient to maintain a high degree of affinity to the target object while reducing the risk of an adverse immune response when administered to the target species. In particularly useful embodiments, the engineered antibodies are humanized.

Technical and scientific terms used herein have the meanings commonly understood by one of ordinary skill in the art to which the present teachings pertain, unless otherwise defined herein. Reference is made herein to various methodologies known to those of skill in the art.

Publications and other materials setting forth such known methodologies to which reference is made are incorporated herein by reference in their entireties as though set forth in full. Practice of the methods described herein will employ, unless otherwise indicated, conventional techniques of chemistry, molecular biology, microbiology, recombinant DNA, and immunology, which are within the skill of the art. Such conventional techniques are explained fully in the literature. See, e.g., Sambrook, Fritsch, and Maniatis, Molecular Cloning; Laboratory Manual 2^{nd} ed. (1989); DNA Cloning, Volumes 1 and 11 (D. N Glover ed. 1985); Oligonucleotide Synthesis (M. J. Gait ed, 1984); Nucleic Acid Hybridization (B. D. Hames & S. J. Higgins eds. 1984); the series, Methods in Enzymology (Academic Press, Inc.), particularly Vol. 154 and Vol. 155 (Wu and Grossman, eds.); PCR-A Practical Approach (McPherson, Quirke, and Taylor, eds., 1991); Immunology, 2d Edition, 1989, Roitt et al., C. V. Mosby Company, and New York; Advanced Immunology, 2d Edition, 1991, Male et al., Grower Medical Publishing, New York; DNA Cloning: A Practical Approach, Volumes I and II, 1985 (D. N. Glover ed.); Oligonucleotide Synthesis, 1984, (M. L. Gait ed); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; and Gene Transfer Vectors for Mammalian Cells, 1987 (J. H. Miller and M. P. Calos eds., Cold Spring Harbor Laboratory); WO97/08320; U.S. Pat. Nos. 5,427,908; 5,885,793; 5,969,108; 5,565,332; 5,837,500; 5,223,409; 5,403,484; 5,643,756; 5,723,287; 5,952,474; Knappik et al., 2000, J. Mol. Biol. 296:57-86; Barbas et al., 1991, Proc. Natl. Acad. Sci. USA 88:7978-7982; Schaffitzel et al. 1999, J. Immunol. Meth. 10:119-135; Kitamura, 1998, Int. J. Hematol., 67:351-359; Georgiou et al., 1997, Nat. Biotechnol. 15:29-34; Little, et al., 1995, J. Biotech. 41:187-195; Chauthaiwale et al., 1992, Microbiol. Rev., 56:577-591; Aruffo, 1991, Curr. Opin. Biotechnol. 2:735-741; McCafferty (Editor) et al., 1996, Antibody Engineering: A Practical Approach, the contents of which are incorporated herein by reference.

Any suitable materials and/or methods known to those of skill can be utilized in carrying out the methods described herein; however, preferred materials and/or methods are described. Materials, reagents and the like to which reference is made in the following description and examples are obtainable from commercial sources, unless otherwise noted. It should be understood that the terms “including”, “included”, “includes” and “include” are used in their broadest sense, i.e., they are open ended and mean, e.g., including but not limited to, included but limited to, includes but not limited to, and include but not limited to.

The engineered antibodies and antibody fragments include complete antibody molecules having full length heavy and light chains, or any fragment thereof, such as Fab, F(ab′)₂, Fd, scFv, diabodies, antibody light chains and antibody heavy chains.

As an initial matter, a predetermined target object is chosen to which an antibody is raised. Techniques for generating polyclonal and monoclonal antibodies directed to target objects are well known to those skilled in the art. Target objects include any substance which is capable of exhibiting antigenicity and are usually proteins or protein polysaccharides. Examples include receptors, enzymes, hormones, growth factors, peptides and the like. It should be understood that not only are naturally occurring antibodies suitable for use in accordance with the present disclosure, but engineered antibodies and antibody fragments which are directed to a predetermined object are also suitable.

Antibodies (Abs) that can be subjected to the techniques set forth herein include monoclonal Abs, and antibody fragments such as Fab, Fab′, F(ab′)₂, Fd, scFv, diabodies, antibody light chains, antibody heavy chains and/or antibody fragments derived from phage or phagemid display technologies. Functional antibody fragments are those fragments of antibodies which are capable of binding to an antigen notwithstanding the absence of regions normally found in whole antibodies. Single chain antibodies (scFv) are included in functional antibody fragments.

Once antibodies to a particular target are identified, they can be engineered to have desirable characteristics. In one embodiment, the antibodies are engineered to include at least a CDR3 from a non-target species, as described below. This embodiment is referred to hereinafter as optimized engineered antibodies. In certain aspects the optimized antibodies may be humanized antibodies. In another embodiment, the antibodies are engineered to include CDRs from at least two sources, which are incorporated into a human framework that is selected using a consensus sequence, as described below. The engineered antibodies of this embodiment are referred to as “humanized antibodies”.

Optimized Antibodies

To begin the optimization process, the DNA sequence of the variable portion of the light and heavy chain genes of an originating species antibody having specificity for a target antigen is needed. The originating species is any species which was used to generate the antibodies, e.g., mice, rabbit, chicken, monkey, etc. Techniques for generating polyclonal and monoclonal antibodies are well known to those skilled in the art. Once that has been obtained, the selection of an appropriate target species framework is necessary. One embodiment involves alignment of the first antibody sequence with germline variable genes from the target species. For example, if the target species is human, a source of such gene sequences may be found in any suitable library such as VBASE, a database of human antibody genes (http://www.mrc-cpe.cam.ac.uk/imt-doc) or translated products thereof. Alternatively, when the target species is again human and the first antibody is non-human one can align the non-human genes to human rearranged antibody sequences, such as those found in the Kabat database of immunoglobulins (http://www.immuno.bme.nwu.edu). If the alignments are done based on the nucleotide sequences, then the selected genes should be analyzed to determine which genes of that subset have the closest amino acid homology to the first antibody. It is contemplated that genes which approach a higher degree homology as compared to other genes in the library can be utilized and manipulated in accordance with the procedures described herein. Moreover, genes which have lesser homology can be utilized when they encode products which, when manipulated and selected in accordance with the procedures described herein, exhibit specificity for the predetermined target object. In certain embodiments, an acceptable range of homology is greater than about 50%. An alternate approach is to take the set of homologous human genes as determined from, e.g., VBASE, the Kabat database and/or the translated products of such databases, and use the consensus amino acid sequence as the human framework. Alignment strategies for the framework 4 region (J gene) can likewise be done using either germline or rearranged antibody sequences. It should be understood that target species may be other than human.

CDRs being incorporated into the target species framework include non-target species CDR3 and a choice in the CDR2 and/or CDR1 between the target species and originating species sequences. The framework residues are generally assigned as target species sequence except at particular positions. For example, non-homologous amino acid residues at either VH/VL interface or Vernier zone positions are maintained as a choice between target species and originating species in the construction of an optimized Fab library. Additional framework positions where a residue choice may be desirable include those highly conserved among the originating species antibodies. In general, it is desirable to keep the surface residues from the target species to further avoid potential immunogenicity of the optimized antibody. However, some surface exposed residues are also designated as VHNL interface or Vernier zones. In that case, choice of either originating species or target species is still given.

Assembly of an optimized antibody or antibody fragment can be accomplished using conventional methods known to those skilled in the art. For example, DNA sequences encoding the altered variable domains described herein may be produced by oligonucleotide synthesis. Alternatively, nucleic acid encoding altered variable domains as described herein may be constructed by primer directed oligonucleotide site-directed mutagenesis, i.e., hybridizing an oligonucleotide coding for a desired mutation with a single nucleic acid strand containing the region to be mutated and using the single strand as a template for extension of the oligonucleotide to produce a strand containing the mutation. The oligonucleotides used for site directed mutagenesis may be prepared by oligonucleotide synthesis or may be isolated from nucleic acid encoding the target species framework by use of suitable restriction enzymes. The nucleic acid encoding CDR regions may be isolated from the originating species antibodies using suitable restriction enzymes and ligated into the target species framework by ligating with suitable ligation enzymes.

Assembly of the antibody fragment library is preferably accomplished using synthetic oligonucleotides. In one example, oligonucleotides were designed to have overlapping regions so that they could anneal and be filled in by a polymerase, such as with polymerase chain reaction (PCR). Multiple steps of overlap extension were performed in order to generate the VL and VH gene inserts. Those fragments were designed with regions of overlap with human constant domains so that they could be fused by overlap extension to produce full length light chains and Fd heavy chain fragments. The light and heavy Fd genes were then fused together by overlap extension to create a single Fab library insert to be cloned into a display vector. Alternative methods for the assembly of the humanized library genes can also be used. For example, the library may be assembled from overlapping oligonucleotides using a Ligase Chain Reaction (LCR) approach. See, e.g., Chalmers and Curnow, Biotechniques (2001) 30-2, p249-252. This LCR technique involves annealing a series (up to 12 optimally) of overlapping oligonucleotides together in such a way as to provide a double stranded gene with only nicks between the oligos (no gaps). Oligonucleotides are ligated together by a thermostable ligase using a thermocycling regiment. Following gene assembly, a round of PCR is performed as there will be redundancy designed into the oligonucleotides and the PCR ensures the production of clonal transformants. A gene could also be assembled from multiple initial LCR fragments by joining them together using PCR overlap extension. Lastly, it is possible to develop antibody variable gene library fragments with different combinations of either murine or human CDR1s and CDR2s with the LCR protocol, by combining different CDR encoding oligos in separate synthesis mixtures.

Various forms of antibody fragments may be generated and cloned into an appropriate vector to create an optimized antibody library. For example variable genes can be cloned into a vector that contains, in-frame, the remaining portion of the necessary constant domain. Examples of additional fragments that can be cloned include whole light chains, the Fd portion of heavy chains, or fragments that contain both light chain and heavy chain Fd coding sequence. Alternatively, the antibody fragments used for humanization may be single chain antibodies (scFv).

Any selection display system may be used in conjunction with a library according to the present disclosure. Selection protocols for isolating desired members of large libraries are known in the art, as typified by phage display techniques. Such systems, in which diverse peptide sequences are displayed on the surface of filamentous bacteriophage (Scott and Smith (1990) Science, 249: 386), have proven useful for creating libraries of antibody fragments (and the nucleotide sequences that encode them) for the in vitro selection and amplification of specific antibody fragments that bind a target antigen. The nucleotide sequences encoding the V_{H} and V_{L} regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage-based display systems is that, because they are biological systems, selected library members can be amplified simply by growing the phage containing the selected library member in bacterial cells. Furthermore, since the nucleotide sequence that encode the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al (1990) Nature, 348: 552; Kang et al. (1991) Proc. Natl. Acad. Sci U.S.A., 88: 4363; Clackson et al. (1991) Nature, 352: 624; Lowman et al., (1991) Biochemistry, 30: 10832; Burton et al. (1991) Proc. Natl. Acad. Sci U.S.A., 88:10134; Hoogenboom et al. (1991) Nucleic Acids Res., 19:4133; Chang et al. (1991) J. Immunol., 147: 3610; Breitling et al. (1991) Gene, 104:147; Marks et al (1991) J. Mol. Biol., 222: 581; Barbas et al. (1992) Proc. Natl. Acad. Sci USA, 89: 4457; Hawkins and Winter (1992) J. Immunol., 22: 867; Marks et al., 1992, J. Biol. Chem., 267:16007; Lerner et al. (1992) Science, 258: 1313, incorporated herein by reference).

One particularly advantageous approach has been the use of scFv phage-libraries (Huston et al., 1988, Proc. Natl. Acad. Sci U.S.A., 85: 5879-5883; Chaudhary et al. (1990) Proc. Natl. Acad. Sci U.S.A., 87:1066-1070; McCafferty et al. (1990) supra; Clackson et al. (1991) supra; Marks et al. (1991) supra; Chiswell et al. (1992) Trends Biotech., 10: 80; Marks et al. (1992) J. Biol. Chem., 267:16007). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys), which are incorporated herein by reference. The display of Fab libraries is also known, for instance as described in WO92/01047 (CAT/MRC) and WO91/17271 (Affymax).

Other systems for generating libraries of antibodies or polynucleotides involve the use of cell-free enzymatic machinery for the in vitro synthesis of the library members. In one method, RNA molecules are selected by alternate rounds of selection against a target ligand and PCR amplification (Tuerk and Gold (1990) Science, 249: 505; Ellington and Szostak (1990) Nature, 346:818). A similar technique may be used to identify DNA sequences which bind a predetermined human transcription factor (Thiesen and Bach (1990) Nucleic Acids Res., 18: 3203; Beaudry and Joyce (1992) Science, 257: 635; WO92/05258 and WO92/14843). In a similar way, in vitro translation can be used to synthesize antibody molecules as a method for generating large libraries. These methods which generally comprise stabilized polysome complexes, are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax) use polysomes to display antibody molecules for selection. These and all the foregoing documents also are incorporated herein by reference.

Humanized Antibodies

In another aspect, the techniques described herein provide humanized composite engineered antibodies which are highly active against predetermined targets and which reduce the risk of immunogenicity in humans. As more fully described below, the humanized antibodies are derived by first determining the sequences of two or more antibodies from a panel of non-human antibodies which demonstrate high binding affinity to a particular target as determined, for example, by panning techniques. An antibody consensus sequence for the two or more members from the panel of non-human antibodies is established by comparing the sequences. A CDR3 from an individual member of the panel of non-human antibodies is substituted for the CDR3 of the consensus sequence to form a composite sequence. This composite sequence is used as the “query” in a comparison (normally achieved by a computer) of the composite sequence to a database of human antibody sequences. At least one human antibody sequence is selected based on homology to the composite sequence. The human antibody sequence or sequences selected provide the framework regions for the engineered composite humanized antibody prepared in accordance with this disclosure. The CDRs for the composite humanized antibody prepared in accordance with this disclosure are selected from at least two sources. Specifically, the CDR3 is selected from an individual member of the panel of non-human antibodies and the CDR1 and CDR2 or both are selected from one or more different sources including other members of the panel, the consensus sequence or the human sequence or sequences identified base on homology to the composite sequence. The resulting composite humanized antibody is thus engineered to have high binding affinity to the target and reduced immunogenicity in humans.

To begin with, a plurality of initial antibodies is obtained from one or more originating non-human species. More particularly, the nucleic acid or amino acid sequences of the variable portion of the light chain, heavy chain or both, of at least two antibodies having specificity for a target antigen are needed. The originating species is any species which was used to generate the antibodies or antibody libraries, e.g., rat, mice, rabbit, chicken, sheep, monkey, human, etc. Techniques for generating and cloning monoclonal antibodies are well known to those skilled in the art. After two or more desired antibodies are obtained, the sequence of each of the antibodies is determined, i.e., the variable regions (VH and VL) may be identified by component parts (i.e., frameworks (FRs), CDRs, Vernier zone regions and VHNL interface regions) using any possible definition of CDRs (e.g., Kabat alone, Chothia alone, Kabat and Chothia combined, and any others known to those skilled in the art) and thus identified.

According to the present humanization methods, at least two antibodies, and preferably more, from one or more originating species are chosen based on a number of criteria including high affinity, specificity and/or activity for the target and high expression. Collectively, the selected antibodies having the desired characteristics are referred to herein as a panel of antibodies. Screening methods for isolating antibodies with high and higher affinity for a target are well-known in the art. For example, the expression of polypeptides fused to the surface of filamentous bacteriophage provides a powerful method for recovering a particular sequence from a large ensemble of clones (Smith et al., Science, 228:1315-1517, 1985). Antibodies binding to peptides or proteins have been selected from large libraries by relatively simple panning methods, e.g., Scott et al., Science, 249:386-290,1990; Devlin et al., Science, 249:404-406, 1990; Cwirla et al., Proc. Natl. Acad. Sci. U.S.A, 87:6378-6382, 1990; McCafferty et al., Nature, 348:552-554,1990; Lowman et al., Biochemistry, 30:10832-10838,1992; and Kang et al., Proc. Natl. Acad. Sci. U.S.A., 88:4363-4366,1991. A variety of techniques are known for display of antibody libraries including phage display, phagemid display, ribosomal display and cell surface display. In panning methods useful to screen antibodies, the target ligand can be immobilized, e.g., on plates, beads, such as magnetic beads, sepharose, etc., beads used in columns. In particular embodiments, the target ligand can be “tagged”, e.g., using such as biotin, 2-fluorochrome, e.g., for FACS sorting.

Screening a library of phage or phagemid expressing antibodies utilizes phage and phagemid vectors where antibodies are fused to a gene encoding a phage coat protein. Target ligands are conjugated to magnetic beads according to manufacturers' instructions. To block non-specific binding to the beads and any unreacted groups, the beads may be incubated with excess BSA. The beads are then washed with numerous cycles of suspension in PBS-0.05% Tween 20 and recovered with a strong magnet along the sides of a plastic tube. The beads are then stored with refrigeration until needed. In the screening experiments, an aliquot of the library may be mixed with a sample of resuspended beads. The tube contents are tumbled at cold temperatures (e.g., 4-5° C.) for a sufficient period of time (e.g., 1-2 hours). The magnetic beads are then recovered with a strong magnet and the liquid is removed by aspiration. The beads are then washed by adding PBS-0.05% Tween 20, inverting the tube several times to resuspend the beads, and then drawing the beads to the tube wall with the magnet. The contents are then removed and washing is repeated 5-10 additional times. 50 mM glycine-HCl (pH 2.2), 100 μg/ml BSA solution are added to the washed beads to denature proteins and release bound phage. After a short incubation time, the beads are pulled to the side of the tubes with a strong magnet and the liquid contents are then transferred to clean tubes. 1 M Tris-HCl (pH 7.5) or 1 M NaH₂PO₄ (pH 7) is added to the tubes to neutralize the pH of the phage sample. The phage are then diluted, e.g., 10⁻³ to 10⁻⁶, and aliquots plated with E. coli cells to determine the number of plaque forming units of the sample. In certain cases, the platings are done in the presence of XGal and IPTG for color discrimination of plaques (i.e., lacZ+ plaques are blue, lacZ-plaques are white). The titer of the input samples is also determined for comparison (dilutions are generally 10⁻⁶ to 10⁻⁹).

Alternatively, screening a library of phage expressing antibodies can be achieved, e.g., as follows using microtiter plates. Target ligand is diluted, e.g., in 100 mM NaHCO₃, pH 8.5 and a small aliquot of ligand solution is adsorbed onto wells of microtiter plates (e.g. by incubation overnight at 4° C.). An aliquot of BSA solution (1 mg/ml, in 100 mM NaHCO₃, pH 8.5) is added and the plate incubated at room temperature for 1 hr. The contents of the microtiter plate are removed and the wells washed carefully with PBS-0.05% Tween 20. The plates are washed free of unbound targets repeatedly. A small aliquot of phage solution is introduced into each well and the wells are incubated at room temperature for 1-2 hrs. The contents of microtiter plates are removed and washed repeatedly. The plates are incubated with wash solution in each well for 20 minutes at room temperature to allow bound phage with rapid dissociation constants to be released. The wells are then washed multiple, e.g., 5, times to remove all unbound phage. To recover the phage bound to the wells, a pH change may be used. An aliquot of 50 mM glycine-HCl (pH 2.2), 100 μ/ml BSA solution is added to washed wells to denature proteins and release bound phage. After 5-10 minutes, the contents are then transferred into clean tubes, and a small aliquot of 1 M Tris-HCl (pH 7.5) or 1 M NaH₂PO₄ (pH 7) is added to neutralize the pH of the phage sample. The phage are then diluted, e.g., 10⁻³ to 10⁻⁶, and aliquots plated with E. coli cells to determine the number of the plaque forming units of the sample. In certain cases, the platings are done in the presence of XGal and IPTG for color discrimination of plaques (i.e., lacz+ plaques are blue, lacZ− plaques are white). The titer of the input samples is also determined for comparison (dilutions are generally 10⁻⁶ to 10⁻⁹).

According to another alternative method, screening a library of antibodies can be achieved using a method comprising a first “enrichment” step and a second filter lift step as follows. Antibodies from an expressed combinatorial library (e.g., in phage) capable of binding to a given ligand (“positives”) are initially enriched by one or two cycles of affinity chromatography. A microtiter well is passively coated with the ligand of choice (e.g., about 10 μg in 100 μl). The well is then blocked with a solution of BSA to prevent non-specific adherence of antibodies to the plastic surface. About 10¹¹ particles expressing antibodies are then added to the well and incubated for several hours. Unbound antibodies are removed by repeated washing of the plate, and specifically bound antibodies are eluted using an acidic glycine-HCl solution or other elution buffer. The eluted antibody phage solution is neutralized with alkali, and amplified, e.g., by infection of E. coli and plating on large petri dishes containing broth in agar. Amplified cultures expressing the antibodies are then titered and the process repeated. Alternatively, the ligand can be covalently coupled to agarose or acrylamide beads using commercially available activated bead reagents. The antibody solution is then simply passed over a small column containing the coupled bead matrix which is then washed extensively and eluted with acid or other eluant. In either case, the goal is to enrich the positives to a frequency of about >1/10⁵. Following enrichment, a filter lift assay is conducted. For example, when antibodies are expressed in phage, approximately 1-2×10⁵ phage are added to 500 μl of log phase E. coli and plated on a large LB-agarose plate with 0.7% agarose in broth. The agarose is allowed to solidify, and a nitrocellulose filter (e.g., 0.45μ) is placed on the agarose surface. A series of registration marks is made with a sterile needle to allow re-alignment of the filter and plate following development as described below. Phage plaques are allowed to develop by overnight incubation at 37° C. (the presence of the filter does not inhibit this process). The filter is then removed from the plate with phage from each individual plaque adhered in situ. The filter is then exposed to a solution of BSA or other blocking agent for 1-2 hours to prevent non-specific binding of the ligand (or “probe”). The probe itself is labeled, for example, either by biotinylation (using commercial NHS-biotin) or direct enzyme labeling, e.g., with horse radish peroxidase or alkaline phosphatase. Probes labeled in this manner are indefinitely stable and can be re-used several times. The blocked filter is exposed to a solution of probe for several hours to allow the probe to bind in situ to any phage on the filter displaying a peptide with significant affinity to the probe. The filter is then washed to remove unbound probe, and then developed by exposure to enzyme substrate solution (in the case of directly labeled probe) or further exposed to a solution of enzyme-labeled avidin (in the case of biotinylated probe). Positive phage plaques are identified by localized deposition of colored enzymatic cleavage product on the filter which corresponds to plaques on the original plate. The developed filter is simply realigned with the plate using the registration marks, and the “positive” plaques are cored from the agarose to recover the phage. Because of the high density of plaques on the original plate, it is usually impossible to isolate a single plaque from the plate on the first pass. Accordingly, phage recovered from the initial core are re-plated at low density and the process is repeated to allow isolation of individual plaques and hence single clones of phage.

Screening a library of plasmid vectors expressing antibodies on the outer surface of bacterial cells can be achieved using magnetic beads as follows. Target ligands are conjugated to magnetic beads essentially as described above for screening phage vectors. A sample of bacterial cells containing recombinant plasmid vectors expressing a plurality of antibodies expressed on the surface of the bacterial cells is mixed with a small aliquot of resuspended beads. The tube contents are tumbled at 4° C. for 1-2 hrs. The magnetic beads are then recovered with a strong magnet and the liquid is removed by aspiration. The beads are then washed, e.g., by adding 1 ml of PBS-0.05% Tween 20, inverting the tube several times to resuspend the beads, and drawing the beads to the tube wall with the magnet and removing the liquid contents. The beads are washed repeatedly 5-10 additional times. The beads are then transferred to a culture flask that contains a sample of culture medium, e.g., LB+ ampicillin. The bound cells undergo cell division in the rich culture medium and the daughter cells will detach from the immobilized targets. When the cells are at log-phase, inducer is added again to the culture to generate more antibodies. These cells are then harvested by centrifugation and rescreened. Successful screening experiments are optimally conducted using multiple, e.g., rounds of serial screening. The recovered cells are then plated at a low density to yield isolated colonies for individual analysis. The individual colonies are selected and used to inoculate LB culture medium containing ampicillin. After overnight culture at 37° C., the cultures are then spun down by centrifugation. Individual cell aliquots are then retested for binding to the target ligand attached to the beads. Binding to other beads, having attached thereto, a non-relevant ligand can be used as a negative control.

Alternatively, screening a library of plasmid vectors expressing antibodies on the surface of bacterial cells can be achieved as follows. Target ligand is adsorbed to microtiter plates as described above for screening phage vectors. After the wells are washed free of unbound target ligand, a sample of bacterial cells is added to a small volume of culture medium and placed in the microtiter wells. After sufficient incubation, the plates are washed repeatedly free of unbound bacteria. A large volume, approximately 100 ml of LB+ ampicillin is added to each well and the plate is incubated at 37° C. for 2 hrs. The bound cells undergo cell division in the rich culture medium and the daughter cells detach from the immobilized targets. The contents of the wells are then transferred to a culture flask that contains about 10 ml LB+ ampicillin. When the cells are at log-phase, inducer is added again to the culture to generate more antibodies. These cells are then harvested by centrifugation and rescreened. Screening can be conducted using rounds of serial screening as described above, with respect to screening using magnetic beads.

According to another embodiment, the libraries expressing antibodies as a surface protein of either a vector or a host cell, e.g., phage or bacterial cell can be screened by passing a solution of the library over a column of a ligand immobilized to a solid matrix, such as sepharose, silica, etc., and recovering those phage that bind to the column after extensive washing and elution.

One important aspect of screening the libraries is that of elution. For clarity of explanation, the following is discussed in terms of antibody expression by phage. It is readily understood, however, that such discussion is applicable to any system where the antibodies are expressed on a surface fusion molecule. It is conceivable that the conditions that disrupt the peptide-target interactions during recovery of the phage are specific for every given peptide sequence from a plurality of proteins expressed on phage. For example, certain interactions may be disrupted by acid pH's but not by basic pH's, and vice versa. Thus, variety of elution conditions should be tested (including but not limited to pH 2-3, pH 12-13, excess target in competition, detergents, mild protein denaturants, urea, varying temperature, light, presence or absence of metal ions, chelators, etc.) to compare the primary structures of the antibodies expressed on the phage recovered for each set of conditions to determine the appropriate elution conditions for each ligand/antibody combination. Some of these elution conditions may be incompatible with phage infection because they are bactericidal and will need to be removed by dialysis. The ability of different expressed proteins to be eluted under different conditions may not only be due to the denaturation of the specific peptide region involved in binding to the target but also may be due to conformational changes in the flanking regions. These flanking sequences may also be denatured in combination with the actual binding sequence; these flanking regions may also change their secondary or tertiary structure in response to exposure to the elution conditions (i.e., pH 2-3, pH 12-13, excess target in competition, detergents, mild protein denaturants, urea, heat, cold, light, metal ions, chelators, etc.) which in turn leads to the conformational deformation of the peptide responsible for binding to the target.

It should be understood that any panning method suitable for recovery of antibodies demonstrating high affinity to a target molecule is suitable. After recovery and determination of which antibodies have the desired affinity, activity, specificity and expression, those antibodies make up the panel of antibodies. The sequences of a plurality of members of the panel of antibodies are then determined using any technique known to those skilled in the art. By comparing the sequences of the members of the panel, the antibodies may advantageously be grouped into families of antibodies based on their sequences. Families of antibodies are delineated in the published databases of antibodies and are well known to those skilled in the art.

A consensus sequence is established for the panel members within the family containing the panel member having the highest affinity to the target. To arrive at a consensus sequence given a group of antibody sequences is within the purview of one skilled in the art. In general, the sequences of the individual panel members within the family are compared and the amino acid appearing most frequently at each position along the sequence is assigned to that position within the consensus sequence. Computer programs are commercially available (e.g., from DNASTAR Inc., Madison, Wis.) which will compare a plurality of sequences and automatically provide a consensus sequence.

The consensus sequence is then modified to provide a composite sequence. To modify the consensus sequence, the CDR3 from a particular panel member is substituted for the CDR3 of the consensus sequence. The particular panel member from which the CDR3 is selected can be chosen based upon a number of factors including, but not limited to expression efficiency, affinity to the target, specificity to the target and activity. Techniques for assessing each of these factors are within the purview of one skilled in the art.

After the composite sequence has been determined, a comparison is made between the composite sequence and one or more databases of known human antibody sequences (e.g., germline, rearranged or both). The comparison is made by aligning the composite sequence with sequences in the database(s) and determining the degree of homology between the sequences being compared. Computer programs for searching for alignments are well known in the art, e.g., BLAST and the like. For example, a source of human amino acid sequences or gene sequences may be found in any suitable reference database such as Genbank, the NCBI protein databank (http://ncbi.nlm.nih.gov/BLAST, VBASE, a database of human antibody genes (http://www.mrc-cpe.cam.ac.uk/imt-doc), (germline sequences), and the Kabat database of immunoglobulins (http://www.immuno.bme.nwu.edu) (rearranged sequences) or translated products thereof. If the alignments are done based on the nucleotide sequences, then the selected genes should be analyzed to determine which genes of that subset have the closest amino acid homology to the originating species antibody as described herein. It is contemplated that amino acid sequences or gene sequences which approach a higher degree homology as compared to other sequences in the database can be utilized and manipulated in accordance with the procedures described herein. In certain embodiments, an acceptable range of homology is greater than about 50%. Depending on the source of the panel, a higher homology may be sought. Specifically, for example, where a panel of murine antibodies is used to form the composite sequence, homologies of 60% or more can advantageously be sought. In any event, the human sequences with the highest degree of homology compared with the respective composite sequences are identified. The human sequence(s) chosen will provide at least the framework regions for the engineered composite humanized antibody produced in accordance with this disclosure. At least one non-human CDR (preferably a non-human CDR3) will be positioned among these human framework regions to produce the engineered composite humanized antibody in accordance with this disclosure.

It is also contemplated that more than one human sequences can be chosen to provide different portions of the engineered composite humanized antibody in accordance with this disclosure. In one particularly useful embodiment, one portion of the present engineered composite humanized antibody is derived from a human germline sequence and another portion of the present engineered composite humanized antibody is derived from a re-arranged human antibody sequence. For example, the portion of the composite sequence including FR1, CDR1, FR2, CDR2 and FR3 can be compared to a database of human germline sequences. By aligning that portion of the composite sequence with the sequences in the germline database, a germline sequence with high homology to the portion of the composite sequence can be identified. The germline sequence identified in this manner can provide the FR1, FR2, FR3 (and possibly the CDR1 and/or CDR2 as described in more detail below) for the engineered composite humanized antibody being constructed. Separately, the portion of the composite sequence including FR3, CDR3, FR4 can be compared to a database of sequences of re-arranged human antibodies. By aligning that portion of the composite sequence with the sequences in the database, a sequence with high homology to the portion of the composite sequence can be identified. The sequence identified in this manner can provide the FR3 and FR4 for the engineered composite humanized antibody being constructed.

The next step in constructing a humanized composite antibody or functional antibody fragment involves selecting CDRs to be incorporated into the framework region of the previously selected human sequence(s). The CDRs chosen come from at least two sources. With respect to the CDR3, the CDR3 from a particular panel member is selected. Preferably, the same CDR3 substituted into the consensus sequence is chosen for incorporation into the engineered composite human antibody. As noted above, the particular panel member from which the CDR3 is selected can be advantageously chosen based upon a number of factors including, but not limited to expression efficiency, affinity to the target, specificity to the target and activity. Techniques for assessing each of these factors are within the purview of one skilled in the art.

With respect to CDR1 and CDR2, selection is made form one or more of the following sources. CDR1 and/or CDR2 can be selected from individual panel members, provided that at least one of CDR1 or CDR2 comes from a panel member other than the panel member from which the CDR3 was chosen. Alternatively, CDR1 and/or CDR2 can be selected from the consensus sequence derived from the sequences of a plurality of panel members. As yet another alternative, CDR1 and/or CDR2 can be selected from the human sequences identified by comparison with the composite sequence.

The framework residues are generally assigned as acceptor species sequence except at particular positions. For example, non-homologous amino acid residues at either VHNL interface or Vernier zone positions are maintained as a choice between composite donor and acceptor sequences. In general, it is desirable to keep the surface residues from the acceptor frameworks from the human antibodies to further avoid potential immunogenicity of the humanized composite antibody. However, some surface exposed residues are also designated as VHNL interface or Vernier zones. In that case, choice of either composite donor or acceptor framework sequences is still given.

After selection and assignment of the CDRs into the acceptor framework regions, assembly of a humanized composite antibody or functional antibody fragment can be accomplished using conventional methods known to those skilled in the art. For example, DNA sequences encoding the altered variable domains described herein may be produced by oligonucleotide synthesis. Subsequently, nucleic acid encoding altered variable domains as described herein may be constructed by primer directed oligonucleotide site-directed mutagenesis, i.e., hybridizing an oligonucleotide coding for a desired mutation with a single nucleic acid strand containing the region to be mutated and using the single strand as a template for extension of the oligonucleotide to produce a strand containing the mutation. The oligonucleotides used for site directed mutagenesis may be prepared by oligonucleotide synthesis or may be isolated from nucleic acid encoding the target species framework by use of suitable restriction enzymes.

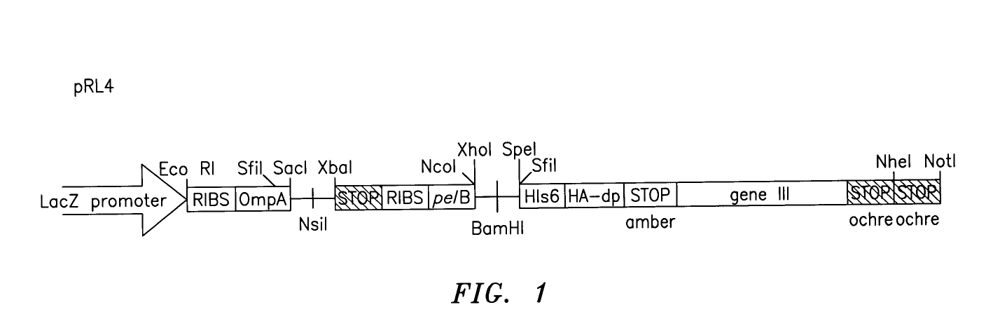

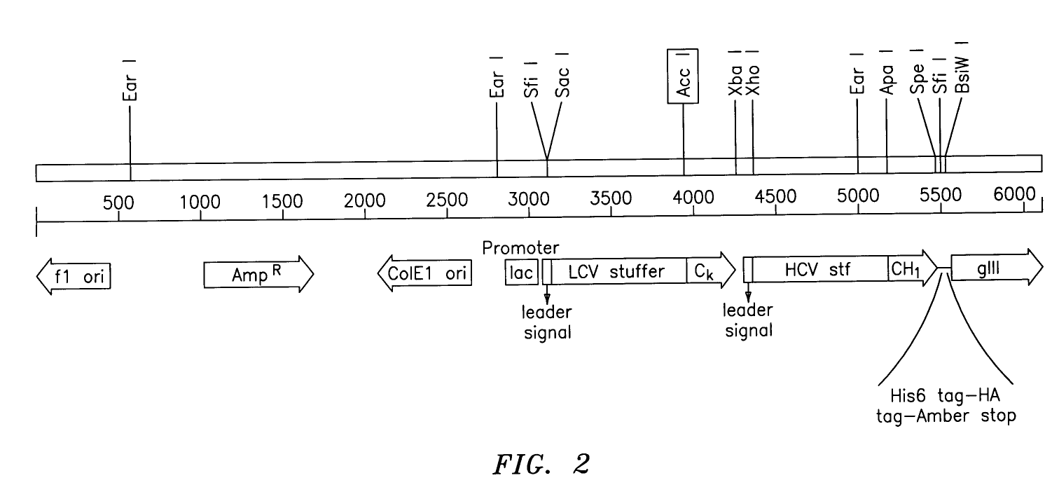

The engineered (either optimized or humanized as described above) antibody or antibody fragments that are cloned into a display vector can be selected against the appropriate antigen in order to identify variants that maintained good binding activity because the antibody will be present on the surface of the phage or phagemid particle. See for example Barbas III, et al. (2001) Phage Display, A Laboratory Manual, Cold Spring Harbor Laboratory Press, Cold Spring Harbor, N.Y., the contents of which are incorporated herein by reference. Although any phage or phagemid display vector would work, vectors such as fdtetDOG, pHEN1, pCANTAB5E, pRL4 (See FIG. 1) or pRL5 (See FIG. 2) are useful for this methodology. For example, in the case of Fab fragments, the light chain and heavy chain Fd products are under the control of a lac promoter, and each chain has a leader signal fused to it in order to be directed to the periplasmic space of the bacterial host. It is in this space that the antibody fragments will be able to properly assemble. The heavy chain fragments are expressed as a fusion with a phage coat protein domain which allows the assembled antibody fragment to be incorporated into the coat of a newly made phage or phagemid particle. Generation of new phagemid particles requires the addition of helper phage which contain all the necessary phage genes. Once a library of antibody fragments is presented on the phage or phagemid surface, a process termed panning follows. This is a method whereby i) the antibodies displayed on the surface of phage or phagemid particles are bound to the desired antigen, ii) non-binders are washed away, iii) bound particles are eluted from the antigen, and iv) eluted particles are exposed to fresh bacterial hosts in order to amplify the enriched pool for an additional round of selection. Typically three or four rounds of panning are performed prior to screening antibody clones for specific binding. In this way phage/phagemid particles allow the linkage of binding phenotype (antibody) with the genotype (DNA) making the use of antibody display technology very successful. However, other vector formats could be used for this humanization process, such as cloning the antibody fragment library into a lytic phage vector (modified T7 or Lambda Zap systems) for selection and/or screening.

After selection of desired engineered antibodies and/or antibody fragments, it is contemplated that they can be produced in large volume by any technique known to those skilled in the art, e.g., in vitro synthesis, recombinant DNA production and the like. For example, antibodies or fragments may be produced by using conventional techniques to construct an expression vector containing an operon that encodes an antibody heavy chain in which CDRs and a minimal portion of the variable region framework that are required to retain donor antibody binding specificity (as engineered according to the techniques described herein) are derived from the originating species antibody and the remainder of the antibody is derived from a target species immunoglobulin which may be manipulated as described herein, thereby producing a vector for the expression of an antibody heavy chain.

Additionally, an expression vector can be constructed which contains an operon that encodes an antibody light chain in which one or more CDRs and a minimal portion of the variable region framework that are required to retain donor antibody binding specificity which may be manipulated as provided herein are derived from the originating species antibody, and the remainder of the antibody is derived from a target species immunoglobulin which can be manipulated as provided herein, thereby producing a vector for the expression of antibody light chain.

The expression vectors may then be transferred to a suitable host cell by conventional techniques to produce a transfected host cell for expression of engineered antibodies or antibody fragments. The transfected host cell is then cultured using any suitable technique known to these skilled in the art to produce antibodies or antibody fragments.

In certain embodiments, host cells may be contransfected with two expression vectors, the first vector containing an operon encoding a heavy chain derived polypeptide and the second containing an operon encoding a light chain derived polypeptide. The two vectors may contain different selectable markers but, with the exception of the heavy and light chain coding sequences, are preferably identical. This procedure provides for equal expression of heavy and light chain polypeptides. Alternatively, a single vector may be used which encodes both heavy and light chain polypeptides. The coding sequences for the heavy and light chains may comprise cDNA or genomic DNA or both.

In certain embodiments, the host cell used to express antibodies or antibody fragments may be either a bacterial cell such as Escherichia coli, or preferably a eukaryotic cell. Preferably a mammalian cell such as a chinese hamster ovary cell or 293EBNA, may be used. The choice of expression vector is dependent upon the choice of host cell, and may be selected so as to have the desired expression and regulatory characteristics in the selected host cell.

Once produced, the engineered antibodies or antibody fragments may be purified by standard procedures of the art, including cross-flow filtration, ammonium sulphate precipitation, affinity column chromatography, gel electrophoresis and the like.

An “anti-PDGF antibody”, as referred to herein, is an immunoglobulin molecule which binds specifically to PDGF. Preferably, in one embodiment the molecule binds to the BB form of PDGF. In another embodiment, the molecule binds to the DD form of PDGF. The anti-PDGF antibody of the invention is preferably a monoclonal anti-PDGF antibody, such as a murine or human monoclonal antibody. In a preferred embodiment, the antibody is a humanized murine antibody. Antibodies may be understood to refer to light chain-heavy chain immunoglobulin tetramers, or fragments thereof which retain antigen binding activity. In one embodiment, the term may also refer to other members of the immunoglobulin superfamily, including T-cell receptors and the like, which may be selected, engineered or otherwise obtained to selectively bind PDGF. Humanized anti-PDGF antibodies may be prepared by the novel method described herein, or, once they have been elucidated, by conventional technology.

A process for the preparation of a hybridoma cell line secreting monoclonal anti-PDGF antibodies can, e.g., involve immunizing a suitable mammal, for example a Balb/c mouse, with one or more PDGF polypeptides or antigenic fragments thereof, or an antigenic carrier containing a PDGF polypeptide; fusing antibody-producing cells of the immunized mammal with cells of a suitable myeloma cell line, cloning the hybrid cells obtained in the fusion, and selecting cell clones secreting the desired antibodies. For example, spleen cells of Balb/c mice immunized with PDGF are fused with cells of the myeloma cell line PAI or the myeloma cell line Sp2/0-Ag14, the obtained hybrid cells are screened for secretion of desired antibodies, and positive hybridoma cells are cloned.

In one example, Balb/c mice are immunized by injecting subcutaneously and/or intraperitoneally between 1 and 100 μg PDGF and a suitable adjuvant, such as Freund's adjuvant, several times, e.g. four to six times, over several months, e.g. between two and four months, and spleen cells from the immunized mice are taken two to four days after the last injection and fused with cells of the myeloma cell line PAI in the presence of a fusion promoter, preferably polyethylene glycol. Preferably the myeloma cells are fused with a three- to twentyfold excess of spleen cells from the immunized mice in a solution containing about 30% to about 50% polyethylene glycol of a molecular weight around 4000. After the fusion, the cells are expanded in suitable culture media as described hereinbefore, supplemented with a selection medium, for example HAT medium, at regular intervals in order to prevent normal myeloma cells from overgrowing the desired hybridoma cells.

Preferred techniques for the generation of antibodies in accordance with the invention include techniques for in vitro isolation of antibody domains from animals immunized with PDGF or antigenic fragments thereof, and selection of antibodies from synthetic libraries constructed using such domains.

RNA may be obtained from spleen and bone marrow cells of immunized mice, for example the use of Tri reagent (Molecular research center, Cincinnati, Ohio, USA). Alternative methods are known in the art and may also be used, examples of which include isolation after treating with guanidine thiocyanate and cesium chloride density gradient centrifugation (Chirgwin, J. M. et al., Biochemistry, 18, 5294-5299, 1979) and treatment with surfactant in the presence of a ribonuclease inhibitor such as vanadium compounds followed by treatment with phenol (Berger, S. L. et al., Biochemistry, 18, 5143-5149, 1979).

In order to obtain single-stranded DNA from RNA, single-stranded DNA complementary to the RNA (cDNA) can be synthesized by using the RNA as a template and treating with reverse transcriptase using oligo(dT) complementary to its polyA chain on the 3′ terminal as primer (Larrik, J. W. et al., Bio/Technology, 7, 934-938, 1989). In addition, a random primer may also be used at that time. Kits for cDNA synthesis are widely available in the art.

Specific amplification of mouse antibody V region genes may be performed from the above-mentioned cDNA using an amplification technique such as the polymerase chain reaction (PCR). Primers such as those described in the Barbas II, et al. (2001) Phage Display, A Laboratory Manual, Cold Spring Harbor Laboratory Press, Cold Spring Harbor, N.Y., or Jones, S. T. et al., Bio/Technology, 9, 88-89, 1991, may be used for amplification of the mouse antibody V region genes. PCR may also be performed with gene-specific primers. An alternative method of amplifying V region genes is described in commonly owned applications entitled “Engineered Templates And Their Use In Single Primer Amplification” and “Nested Oligonucleotides Containing A Hairpin For Nucleic Acid Amplification”, both filed on Sep. 19, 2001, and hereby incorporated by reference.

V region genes may be cloned into phage or phagemids, or another suitable selection system, and presented as a library for selection against PDGF antigen. Library construction and panning techniques are well known in the art.

Nucleic acid encoding a heavy chain variable domain and/or for a light chain variable domain of antibodies directed to PDGF can also be enzymatically or chemically synthesized nucleic acid having the authentic nucleic acid sequence e.g., DNA or RNA, coding for a heavy chain variable domain and/or for the light chain variable domain, or a mutant thereof. A mutant of the authentic nucleic acid is a nucleic acid encoding a heavy chain variable domain and/or a light chain variable domain of the above-mentioned antibodies in which one or more amino acids are deleted or exchanged with one or more other amino acids. Preferably said modification(s) are outside the CDRs of the heavy chain variable domain and/or of the light chain variable domain of the antibody. Such a mutant nucleic acid is also intended to be a silent mutant wherein one or more nucleotides are replaced by other nucleotides with the new codons coding for the same amino acid(s). Such a mutant sequence is also a degenerated sequence. Degenerated sequences are degenerated within the meaning of the genetic code in that an unlimited number of nucleotides are replaced by other nucleotides without resulting in a change of the amino acid sequence originally encoded. Such degenerated sequences may be useful due to their different restriction sites and/or frequency of particular codons which are preferred by the specific host, particularly E. coli, to obtain an optimal expression of the heavy chain murine variable domain and/or a light chain murine variable domain.

The term mutant is intended to include a nucleic acid mutant obtained by in vitro mutagenesis of the authentic nucleic acid according to methods known in the art. For the assembly of complete tetrameric immunoglobulin molecules and the expression of chimeric antibodies, the recombinant nucleic acid inserts coding for heavy and light chain variable domains are fused with the corresponding nucleic acids coding for heavy and light chain constant domains, then transferred into appropriate host cells, for example after incorporation into hybrid vectors.

Recombinant nucleic acid including an insert coding for a heavy chain murine variable domain of an anti-PDGF antibody fused to a human constant domain y, for example y1, y2, y3 or y4, preferably y1 or y4 can be constructed in accordance with the present disclosure by one skilled in the art. Likewise recombinant nucleic acid including an insert coding for a light chain murine variable domain of an anti-PDGF antibody directed to PDGF fused to a human constant domain k or λ, preferably k an also be constructed. Additionally, recombinant nucleic acid can be constructed which codes for a recombinant polypeptide wherein the heavy chain variable domain and the light chain variable domain are linked by way of a spacer group. The nucleic acid may optionally contain a signal sequence facilitating the processing of the antibody in the host cell and/or a nucleic acid coding for a peptide facilitating the purification of the antibody and/or a cleavage site and/or a peptide spacer and/or an effector molecule. The nucleic acid coding for an effector molecule can be a useful in diagnostic or therapeutic applications. Thus, effector molecules which are toxins or enzymes, especially enzymes capable of catalyzing the activation of prodrugs, are particularly indicated. The nucleic acid encoding such an effector molecule has the sequence of a naturally occurring enzyme or toxin, or a mutant thereof, and can be prepared by methods well known in the art.

Once a murine, rabbit or human antibody has been identified, chimeric and reshaped antibodies may be prepared. A chimeric anti-PDGF antibody can be obtained by linking the mouse or rabbit V regions identified by panning with DNA coding for a human antibody constant region and then expressing them.

A basic method for producing chimeric antibodies comprises linking a mouse or rabbit leader sequence and variable region sequence in cloned cDNA with a sequence coding for a human antibody constant region already present in a mammalian cell expression vector. The human antibody constant region can be any human light chain constant region or heavy chain constant region, examples of which include human light chain Ck or heavy chain y C1 or C4. Variable and constant region genes may moreover be joined by overlap PCR, and the gene thus produced cloned into phage display vectors for selection.

In order to produce a chimeric antibody, an expression vector comprising DNA coding for a mouse or rabbit light chain variable region and a human light chain constant region under the control of an expression control region such as a suitable enhancer/promoter, and an expression vector comprising DNA coding for a mouse or rabbit heavy chain variable region and a human heavy chain constant region, also under control of an expression control region, may be used. Host cells such as mammalian cells are co-transformed with these expression vectors and the transformed host is cultured to produce chimeric antibody (for example, see WO91/16928).

Alternatively, DNA coding for a mouse or rabbit light chain variable region and a human light chain constant region, and DNA coding for a mouse or rabbit heavy chain variable region and a human heavy chain constant region may be introduced into a single expression vector, host cells transformed using said vector, and this transformed host cultured to produce the desired chimeric antibody.

CDRs from the mouse or rabbit antibody are grafted into the chosen human framework, for example using suitable oligonucleotides to mutate the human sequences by PCR. At the same time, variation may be introduced into the framework and/or CDR sequences. For example, FR or CDR residues which are known to affect binding site conformation may be varied. Residues which are known to be exposed are preferably mutated to match known or consensus human sequences. Such mutations, each effected independently or together with one or more others, lead to the generation of a further library of antibodies based on the CDRs selected from the anti-PDGF mouse V region genes and a human antibody framework, but comprising mutations introduced therein. Antibodies with particularly favorable properties may be selected from such a library, for example using the selection procedures set forth above.

Highly preferred murine and humanized monoclonal antibodies designated C1 (murine), E1 and B1 (humanized) are set forth in the Figures. C1 was identified by selection from a murine antibody library prepared by amplification of VH and VL regions from mice immunized with human PDGF-BB. Humanization was performed as described above. Thus, in one embodiment, a PDGF antibody herein includes at least one CDR derived from the murine C1 antibody set forth herein. Preferably, at least CDR3 is derived from the C1 antibody. CDRs 1 and 2 may be varied, in accordance with human antibody preference, and suitable antibodies selected, for example by panning a resulting library. Likewise, framework residues known to affect binding site conformation may be varied and successful antibodies selected.

Antibodies as described herein may comprise at least one CDR derived from the B1 or E1 antibodies as set forth herein. Optionally, two or three CDRs may be derived from said antibodies. The CDRs may all be derived from the same antibody, or from different antibodies. It will of course be understood that the CDRs may comprise one or more mutations, introduced as described above; such mutated CDRs are encompassed within the term “derived from”, as used herein.

The anti-PDGF antibodies or anti-PDGF antibody fragments may be used in conjunction with, or attached to other antibodies (or parts thereof) such as human or humanized monoclonal antibodies. These other antibodies may be catalytic antibodies and/or reactive with other markers (epitopes) characteristic for a disease against which the antibodies are directed or may have different specificities chosen, for example, to recruit molecules or cells of the target species, e.g., receptors, target proteins, diseased cells, etc. The antibodies (or parts thereof) may be administered with such antibodies (or parts thereof) as separately administered compositions or as a single composition with the two agents linked by conventional chemical or by molecular biological methods. Additionally the diagnostic and therapeutic value of the antibodies may be augmented by labeling the antibodies with labels that produce a detectable signal (either in vitro or in vivo) or with a label having a therapeutic property. Some labels, e.g. radionucleotides may produce a detectable signal and have a therapeutic property. Examples of radionuclide labels include ¹²⁵I, ¹³¹I, ¹⁴C. Examples of other detectable labels include a fluorescent chromosphere such as green fluorescent protein, fluorescein, phycobiliprotein or tetraethyl rhodamine for fluorescence microscopy, an enzyme which produces a fluorescent or colored product for detection by fluorescence, absorbance, visible color or agglutination, which produces an electron dense product for demonstration by electron microscopy; or an electron dense molecule such as ferritin, peroxidase or gold beads for direct or indirect electron microscopic visualization.

The anti-PDGF antibodies or anti-PDGF antibody fragments herein may typically be administered to a patient in a composition comprising a pharmaceutical carrier. A pharmaceutical carrier can be any compatible, non-toxic substance suitable for delivery of the monoclonal antibodies to the patient, Sterile water, alcohol, fats, waxes, and inert solids may be included in the carrier. Pharmaceutically accepted adjuvants (buffering agents, dispersing agent) may also be incorporated into the pharmaceutical composition. It should be understood that compositions can contain both entire antibodies and antibody fragments.

The engineered antibody and/or fragment compositions in accordance with this disclosure may be administered to a patient in a variety of ways. Preferably, the pharmaceutical compositions may be administered parenterally, e.g., subcutaneously, intramuscularly, epidurally or intravenously. Thus, compositions for parental administration may include a solution of the antibody, antibody fragment, or a cocktail thereof dissolved in an acceptable carrier, preferably an aqueous carrier. A variety of aqueous carriers can be used, e.g., water, buffered water, 0.4% saline, 0.3% glycine and the like. These solutions are sterile and generally free of particulate matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. The concentration of antibody or antibody fragment in these formulations can vary widely, e.g., from less than about 0.5%, usually at or at least about 1% to as much as 15 or 20% by weight and will be selected primarily based on fluid volumes, viscosities, etc., in accordance with the particular mode of administration selected.

In case of brain cancer, pharmaceutical compositions as described herein are preferably capable of crossing the blood-brain barrier. For example, the composition may comprise a brain targeting moiety, such as an anti-insulin receptor antibody (Coloma et al., (2000) Pharm Res 17:266-74), anti-transferrin receptor antibodies (Zhang and Pardridge, (2001) Brain res 889:49-56) or activated T-cells (Westland et al., (1999) Brain 122:1283-91). Alternatively, techniques resulting in modification of the vasculature by the use of vasoactive peptides such as bradykinin or other techniques such as osmotic shock (reviewed in Begley, (1996) J Pharm Pharmacol 48:136-46; Neuwelt et al., (1987) Neurosurgery 20:885-95; Kroll et al., (1998) Neurosurgery 43:879-86; Temsamani et al., (2000) Pharm Sci Technol Today 3:155-162) may be employed.

In a further aspect there is provided the antibodies and/or antibody fragments as hereinbefore defined for use in the treatment of disease. Consequently, there is provided the use of an antibody of the invention for the manufacture of a medicament for the treatment of disease associated with neural cell proliferation or any other condition referred to herein. Cancer which may be treated with antibodies or antibody fragments as described herein include neuroblastoma, neuroepithelioma, meningiomas, Ewing's sarcoma, astrocytoma, glioblastoma, Kaposi's sarcoma, mesothelioma and mesothelioma cell lines, choriocarcinoma, pancreatic carcinoma, gastric carcinoma, osteosarcoma, esophageal cancer, fibrosarcoma, malignant epithelial cells in primary human lung carcinoma, leiomyosarcoma, liposarcoma, paraganglioma, angiosarcoma, hemangiopericytoma, sarcoma NOS, synovial sarcoma, chondrosarcoma, and uterine stromal sarcoma, mammary carcinoma, colorectal cancer, small-cell lung carcinoma, non-small cell lung cancer, malignant fibrous histiocytoma, smooth muscle cell tumor, prostrate cancer. It is contemplated that the antibodies or antibody fragments described herein may be used in combination with other therapeutic agents. Blocking of signaling through the PDGF-β receptor decreases interstitial hypertension in tumors and allows for increasing drug intake, see, e.g., Pietras et al. Cancer Research 61, 2929-34 (2001).

Actual methods for preparing parenterally administrable compositions and adjustments necessary for administration to subjects will be known or apparent to those skilled in the art and are described in more detail in, for example, Remington's Pharmaceutical Science, 17^{th} Ed., Mack Publishing Company, Easton, Pa (1985), which is incorporated herein by reference.

The following examples are provided by way of illustration and should not be construed or interpreted as limiting any of the subject matter described herein.

### EXAMPLE 1

### Humanization of Rabbit F3 Antibody

I. Generation of Rabbit F3 Anti-PDGF Antibody

Rabbit Immunization

One NZW rabbit was immunized with recombinant human platelet derived growth factor (PDGF-BB) (R&D Systems, MN). 15 mg of PDGF-BB in Freund's Complete adjuvant (Sigma, St. Louis, Mo.) was administered to the rabbit sub-cutaneously over 5 to 6 sites in the rear leg. The injections were repeated at three and six weeks after the initial injection using the same dose of PDGF-BB in Freund's Incomplete adjuvant (Sigma, St. Louis, Mo.). The rabbit was bled prior to the first and third injections, and the serum used in an ELISA to monitor the rabbit's anti-PDGF antibody response. Five days after the third injection, the rabbit was sacrificed to collect the spleen, bone marrow, and peripheral blood lymphocytes. RNA was isolated using Tri-reagent (Molecular Research Center, Cincinnati) according to manufacturer's instructions. First-strand cDNA was synthesized using “Superscript Preamplification System for first strand cDNA synthesis kit” with oligo (dT) priming (GibcoBRL, Rockville, Md.) according to manufacturer's instructions.

Antibody Library Construction and Selection

Rabbit VL and VH regions were amplified from the first strand cDNA by 20 cycles of PCR using heavy and light chain primers as described (Barbas, C. F., et al., Phage Display, A Laboratory Manual (2001) Cold Spring Harbor Laboratory Press, Cold Spring Harbor, N.Y.) followed by a fusion PCR reaction to create single chain Fv (scFv) fragments whereby VL and VH were connected with a 21 bp linker having the following sequence 5′ ggt ggt tcc tct aga tct tcc3′ (Seq. Id. No.1). The scFv fragments were cloned into phagemid vector pRL4 using restriction enzyme Sfi I. Transformation into ER2537 cells (New England Biolabs, Beverly, Mass.) yielded a library of 6×10⁷ independent clones. The library was panned 4 times against immobilized recombinant human PDGF-BB antigen (R&D Systems, MN). Microtiter wells were coated with 500 ng antigen/well in the first and second rounds, and 250 ng antigen/well in the third and fourth rounds. The wells were washed 3 times with 0.5% Tween in TBS in the first round, 5 times in the second round and 10 times in the last 2 rounds. The remaining antibody-displaying particles were eluted using 0.1 M HCl pH 2.2 containing 1 mg/ml BSA, followed by neutralization with 2 M Tris. Clones obtained after round 4 were tested for binding to PDGF-BB in solid phase ELISA.

PDGF ELISA

High protein binding 96-well plates (Costar, NY) were coated overnight with 100 ng of recombinant PDGF-BB (R+D Systems, MN) in 0.1 M NaHCO3 pH 8.6 at 4° C. The plates were blocked with 1% BSA for 1 hour at 37° C. followed by 3 washes of PBS/0.05% Tween solution. E. coli culture supernatants or purified antibody was added at various concentrations in PBS/1% BSA for 2 hours at 37° C. on a shaker. After 3 washes with PBS/0.05% Tween, anti-HA primary antibody (12CA5 ascites) at a 1:10,000 dilution in PBS/1% BSA was added and incubated for 2 hours. The plate was washed 3×with PBS/0.05% Tween followed by incubation with alkaline phosphatase-conjugated anti-mouse IgG (Sigma, St. Louis, Mo.) for 2 hours. After incubation, the plates were washed 3× with PBS/0.05% Tween and 3× with PBS. Color development after addition of Sigma 104 substrate in PNPP buffer (10 mM diethanolamine, 0.5 mM MgCL2, pH 9.5) was determined at OD 405 using a microplate reader (Molecular Devices). Anti-PDGF antibodies demonstrating a strong ELISA signal were identified for further analysis to determine which were able to compete with the PDGF-β receptor for binding to PDGF-BB cytokine as described below.

Antibody Purification

Candidate scFvs containing a His 6 purification tag were purified from periplasmic preparations of transfected bacterial cultures using Ni-column chromatography. Periplasmic fractions were obtained by the addition of 20% sucrose in 30 mM Tris pH 8 to the bacterial pellets for 20 minutes. The suspension was spun down for 10 min at 9000 g in 4° C. The supernatant was placed on ice and the pellet was resuspended in sterile cold water and left on ice for 10 min. After centrifugation at 10,000 g, the supernatant was combined with the sucrose supernatant and centrifuged at 12,000 g for 20 minutes. EDTA free Mini protease inhibitor cocktail (Roche Molecular Biochemicals, Indianapolis, Ind.) in 0.2 M NaCl and 10 mM imidazole was prepared and added to the mixture. After filtration, the periplasmic fraction was loaded onto a Ni-charged 5 ml HiTrap chelating column (Amersham Pharmacia, Piscataway, N.J.). The washing buffer used in this purification step contained 20 mM NaH2PO4/Na2HPO4, 0.5 M NaCl and 10 mM imidazole adjusted to pH 7.4. The elution buffer contained 20 mM NaH2PO4/Na2HPO4 0.5 M NaCl and 500 mM imidazole adjusted to pH 7.4. 1 ml fractions were collected and those with elevated OD 280 values were pooled and dialyzed against PBS buffer.

Competition ELISA

The ability of the rabbit scFvs to compete with the PDGF-1 receptor for binding to the PDGF-BB cytokine was determined in a competition assay. Binding of the scFvs to their target epitope on the PDGF-BB cytokine prevents PDGF-BB binding to the PDGF receptor coated on the plates. High protein binding 96-well plates were coated with 10 ng/well of recombinant human PDGF receptor beta/Fc chimera (R&D Systems, MN) in PBS overnight at 4° C. The plates were blocked with 5% sucrose/1% BSA/PBS for 1 h at 37° C. Serial dilutions of purified anti-PDGF-BB antibody were incubated with 10 ng/ml of recombinant human PDGF-BB at room temperature for 30 minutes before being added to the blocked and washed plates. After 2 h at room temperature, the plates were washed with PBS/0.05% Tween followed by another 2-hour incubation with biotinylated anti-PDGF-BB antibody (R&D Systems, MN) at room temperature. Next, streptavidin-alkaline phosphatase (Pierce, Rockford, Ill.) was added to the plates and incubated for 30 minutes at room temperature. The plates were washed 3×with PBS/0.05% Tween and 3× with PBS. Sigma 104 substrate suspended in PNPP buffer was added after the final wash step and the signal at OD405 was determined. Results from the competition assay revealed that clone F3 inhibited PDGF-BB binding to its target receptors. F3 was then selected for humanization.

II. F3 Antibody Humanization

Selection of Homologous Human Sequences

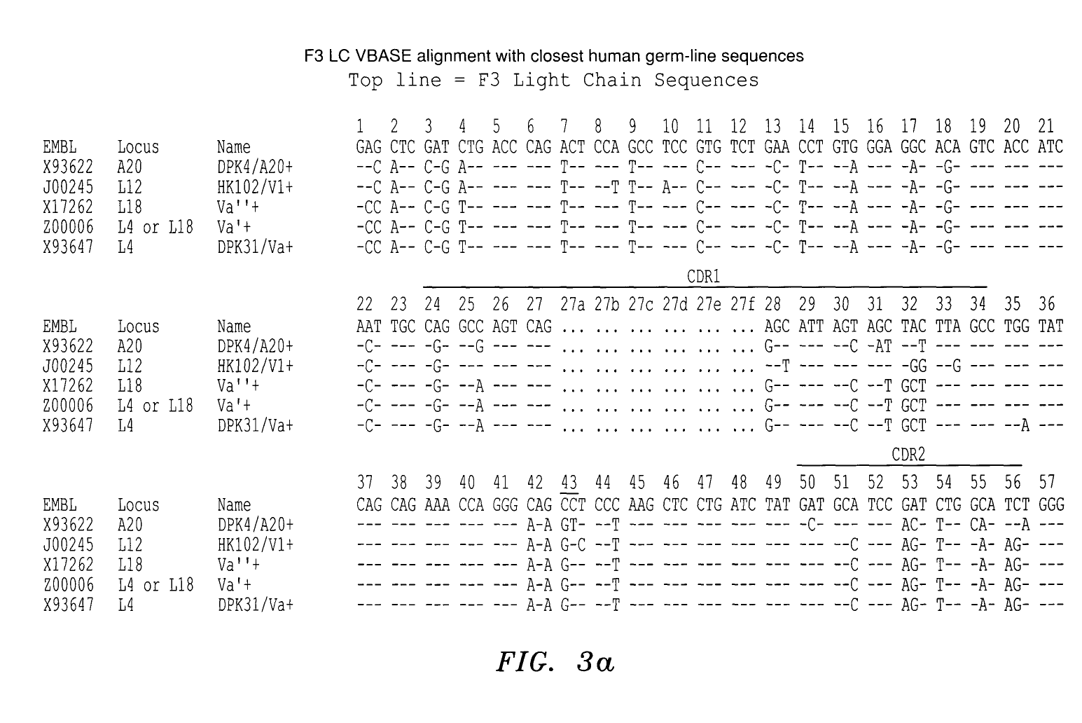

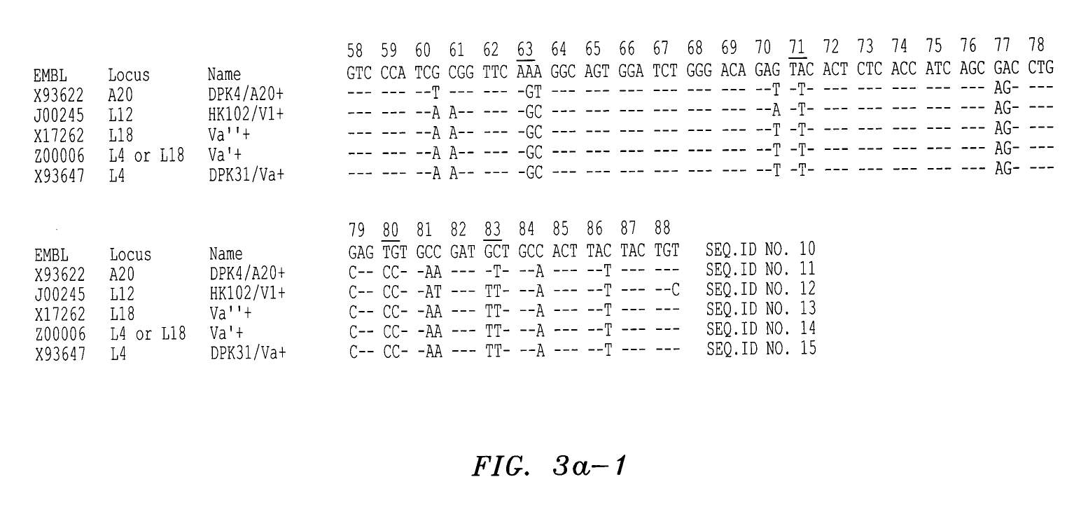

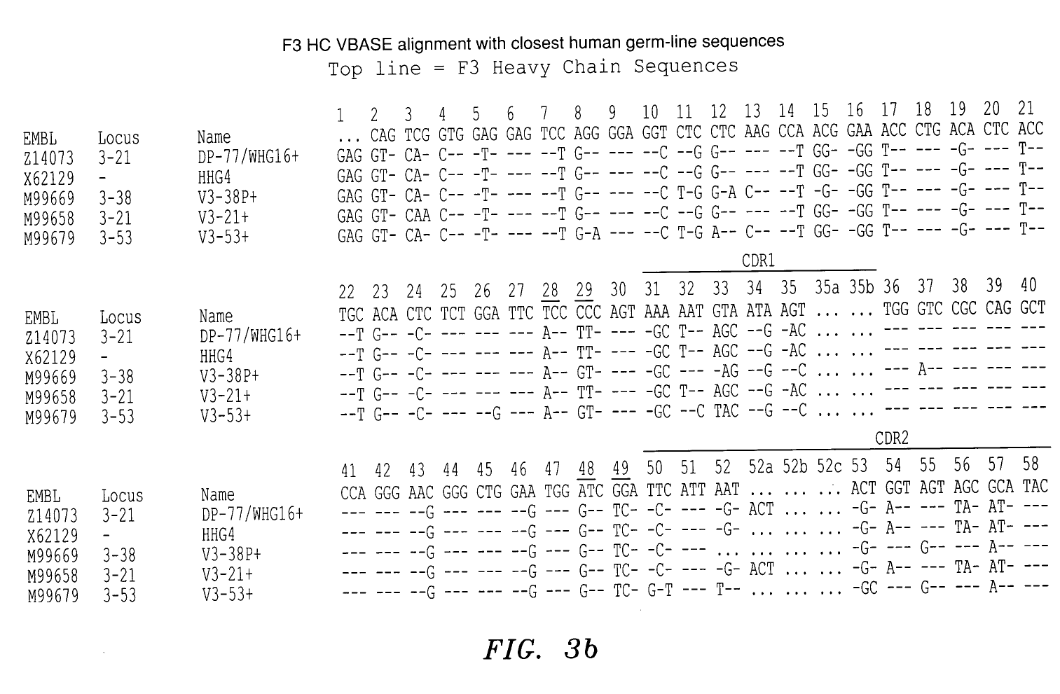

F3's variable genes (FR1, CDR1, FR2, CDR2, and FR3) were aligned against the VBase data bank of human germline sequences (http://www.mrc-cpe.cam.ac.uk/imt-doc). A set of human germline genes were identified which had the highest nucleotide homology to the F3 variable sequences. See FIGS. 3a-3b. Those sequences were further analyzed by determining the amino acid homology between the rabbit and human genes, which identified heavy chain sequence DP-77/WHG16+ and light chain sequence DPK4/A20+ as the germline V-genes to be used in the humanization of F3.

Human FR4 sequences to be used for the humanization were identified based on amino acid homology of F3 sequence with the Kabat database of immunoglobulins located at http://www.immuno.bme.nwu.edu. The sequences identified were Kabat ID: 004927 and 00784.

Identification of Critical Framework Residues

Framework residue positions reported to be involved in either the VH/VL interface or in CDR loop conformation (Vernier zone) were identified. See Table 1 below for a list of certain important residues. Of those positions, four had non-homologous amino acids between the human and rabbit sequences for the light chain and eight were non-homologous for the heavy chain. These framework residues were targeted for diversification such that on construction of the humanized antibody gene, a choice between the rabbit and human residue was given at those positions. See FIGS. 3a and 3b underlined framework residues. However, sometimes in coding for those two amino acids additional choices would result due to the degenerate coding. An additional position for the light chain (residue 63) was targeted for diversification because it was highly conserved in other closely related rabbit sequences from the Kabat database. Some consideration was also given to surface exposed residues, although that assignment is varied depending on the reference used (Studnicka, et al. (1994) Protein Engineering, 7, pp805-814. Pedersen, et al. (1994) J. Mol. Biol., 235, pp959-973.). In general it is desirable to keep the surface residues as human to avoid potential immunogenicity of the humanized antibody. However, some surface exposed residues are also designated as VHNL interface or Vernier zones, such as HC residue 91. In that case, choice of either rabbit or human was still given.

TABLE 1
Important Framework Positions (Kabat numbering)

| | |
|---|---|
| Vernier Zone | VH/VL Interface |

Light Chain

| | |
|---|---|
| 2 | 36 |
| 4 | 38 |
| 35 | 43 |
| 36 | 44 |
| 46 | 46 |
| 47 | 49 |
| 48 | 80 |
| 49 | 83 |
| 64 | 87 |
| 66 | 98 |
| 68 | |
| 69 | |
| 71 | |
| 98 | |

Heavy Chain

| | |
|---|---|
| 2 | 37 |
| 27 | 39 |
| 28 | 45 |
| 29 | 47 |
| 30 | 91 |
| 47 | 93 |
| 48 | 103 |
| 49 | |
| 67 | |
| 69 | |
| 71 | |
| 73 | |
| 78 | |
| 93 | |
| 94 | |
| 103 | |

Oligonucleotide Design

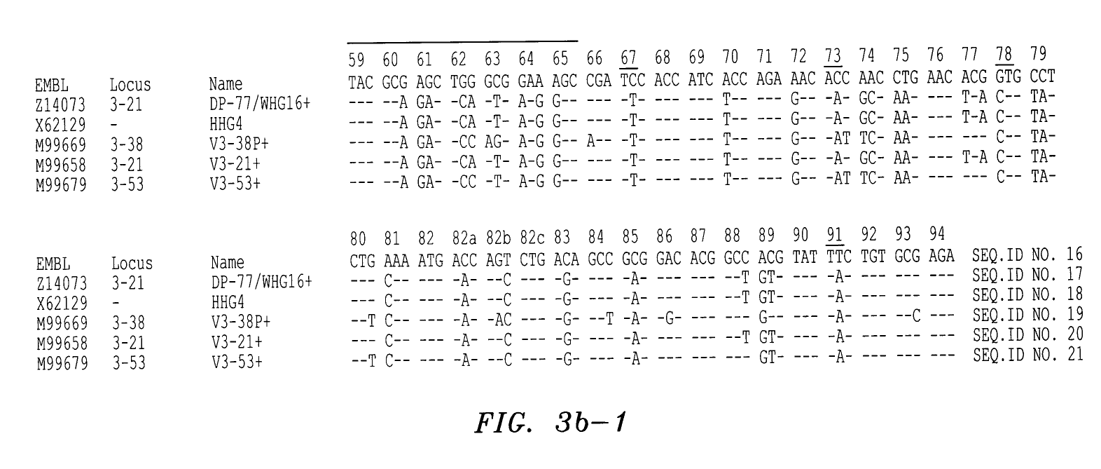

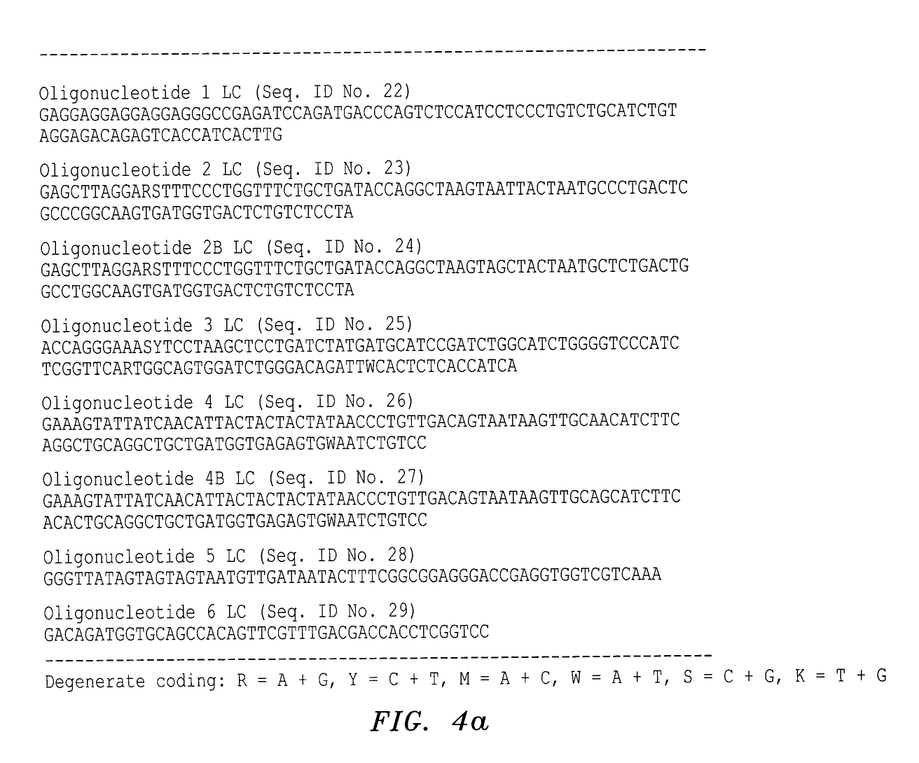

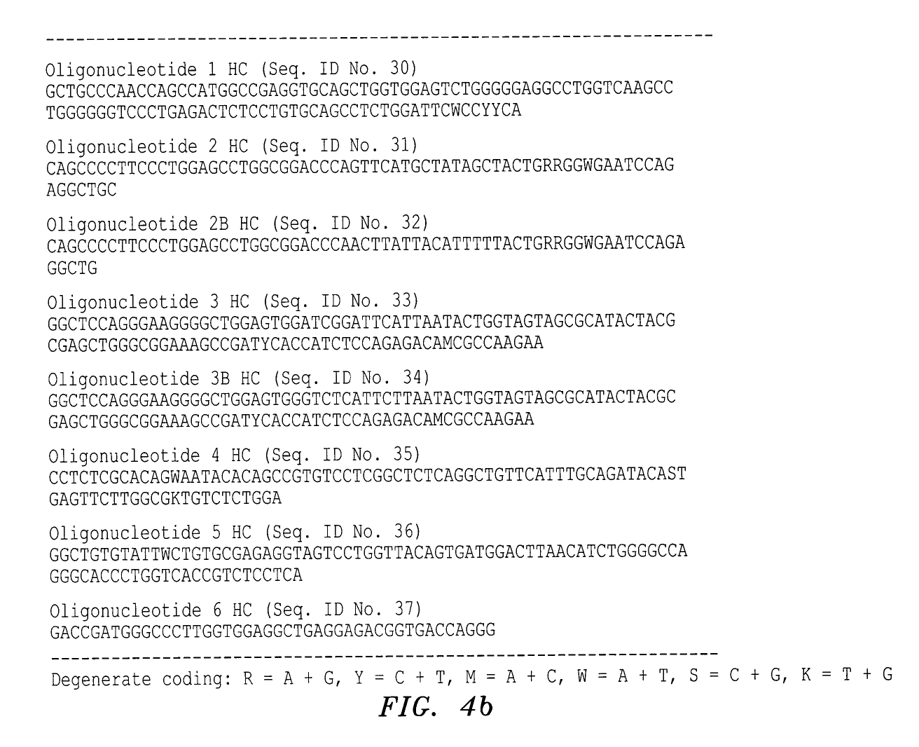

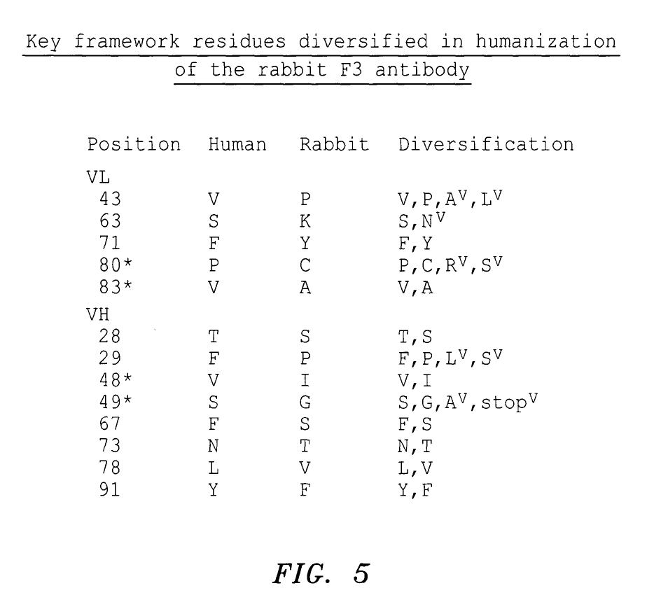

A series of oligonucleotides coding for VL or VH genes were designed which contained overlapping regions to be used for PCR gene assembly (FIGS. 4a, 4b). The oligonucleotides were 80 to 100 basepairs in length and contained degeneracy in designated positions in order to allow amino acid choice (FIG. 5). In addition, oligonucleotides pairs were designed to provide choice of either human or rabbit CDR1. For example, “oligonucleotide 2 HC” provides human CDR1 whereas use of “oligonucleotide 2b HC” results in a rabbit CDR1. Amino acid residues in the CDR 2 and CDR3 were kept as rabbit exclusively. The fully assembled products of these oligonucleotides encode a library of humanized F3 variable domains (VH or VL) containing degenerate amino acid residues in critical framework positions as well as CDR choice in CDR 1. Although in this example choice between human and non-human CDR was given only for CDR1, for other humanization cases it may be desirable to have choice in both CDR1 and CDR2.

Synthetic Assembly of Humanized Rabbit F3 Antibody Library

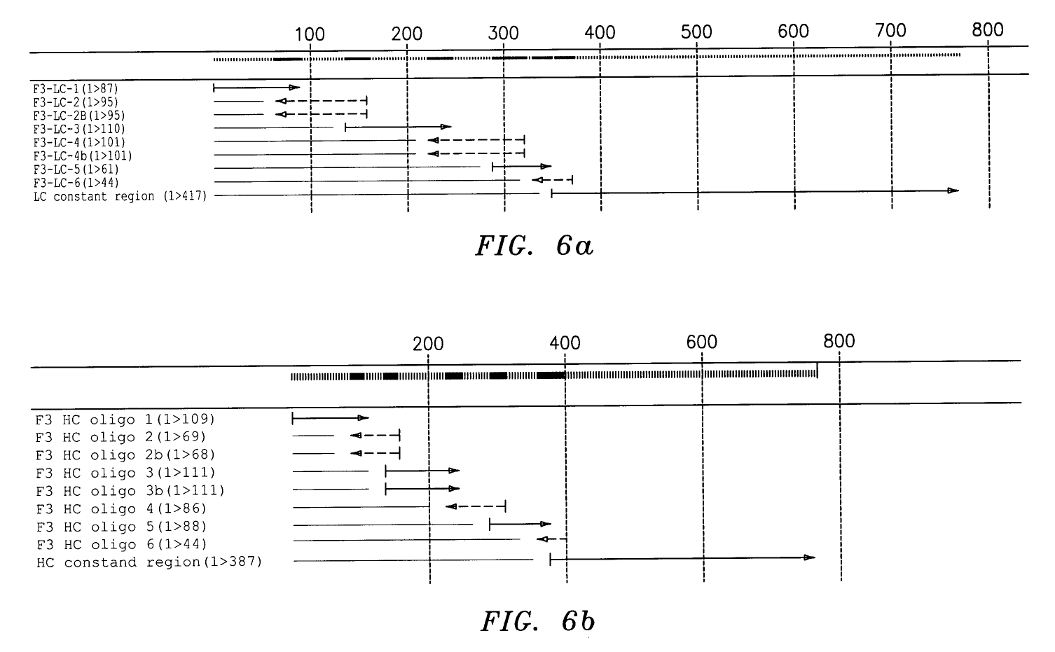

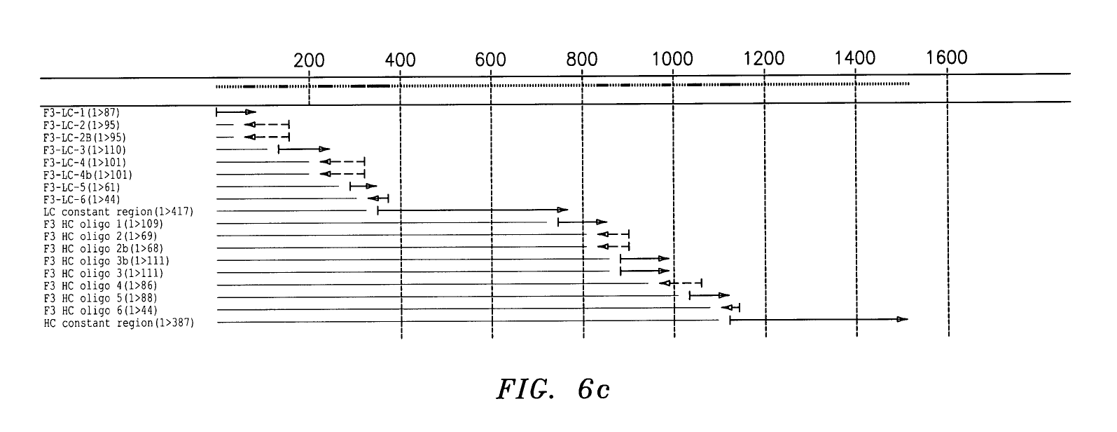

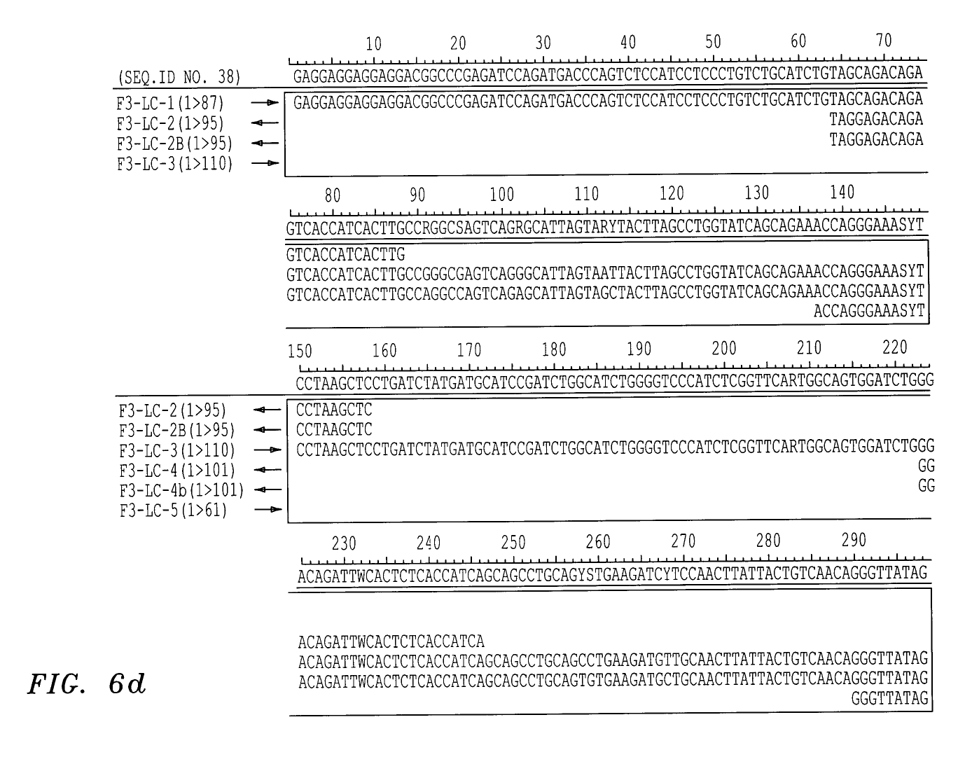

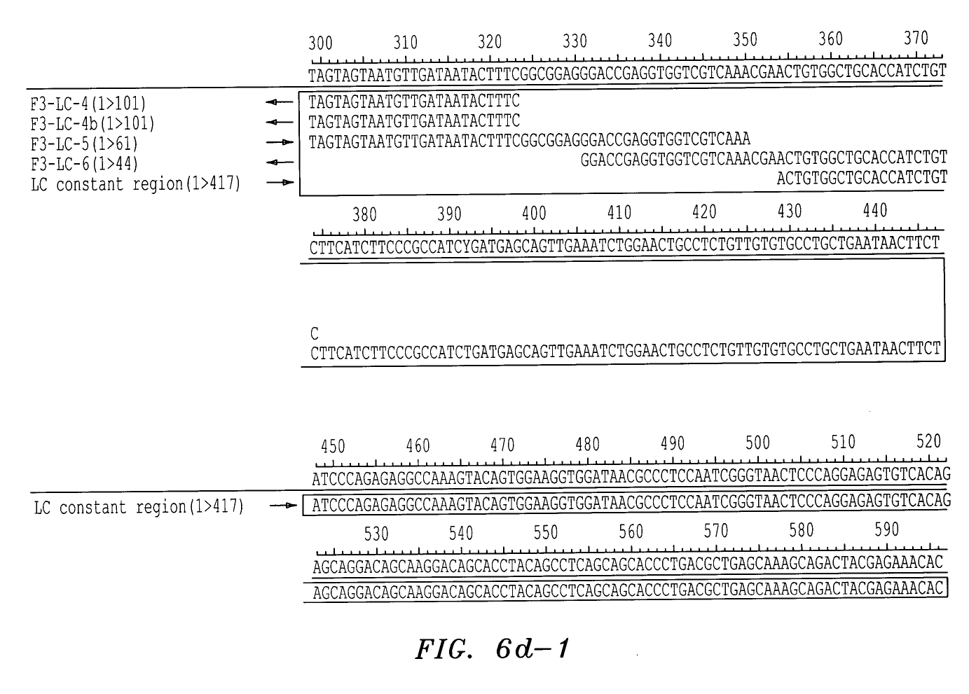

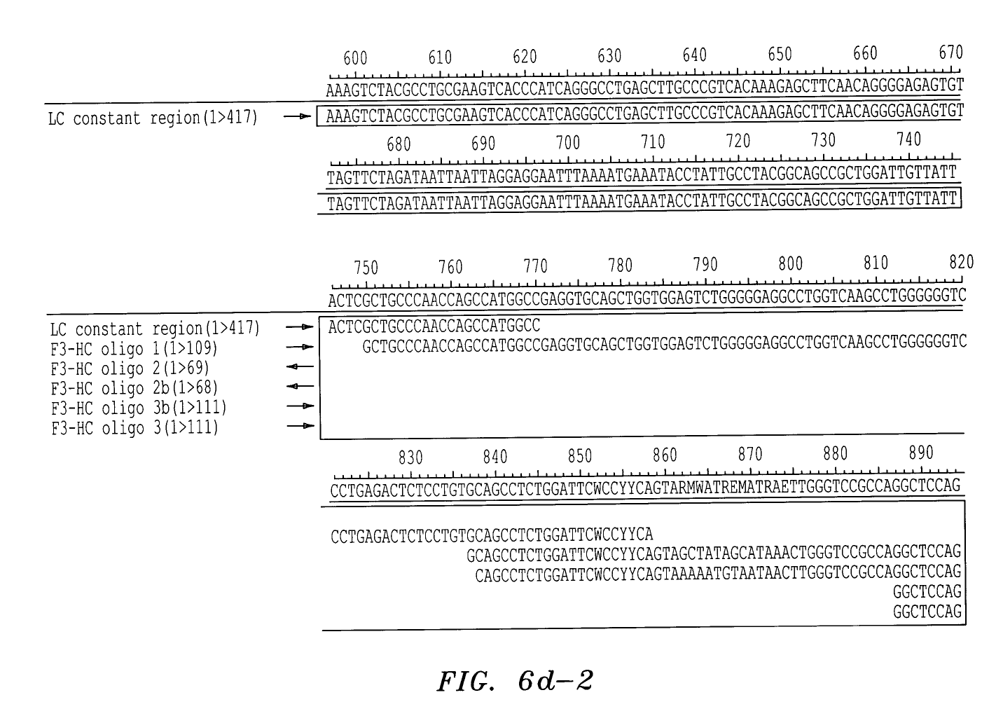

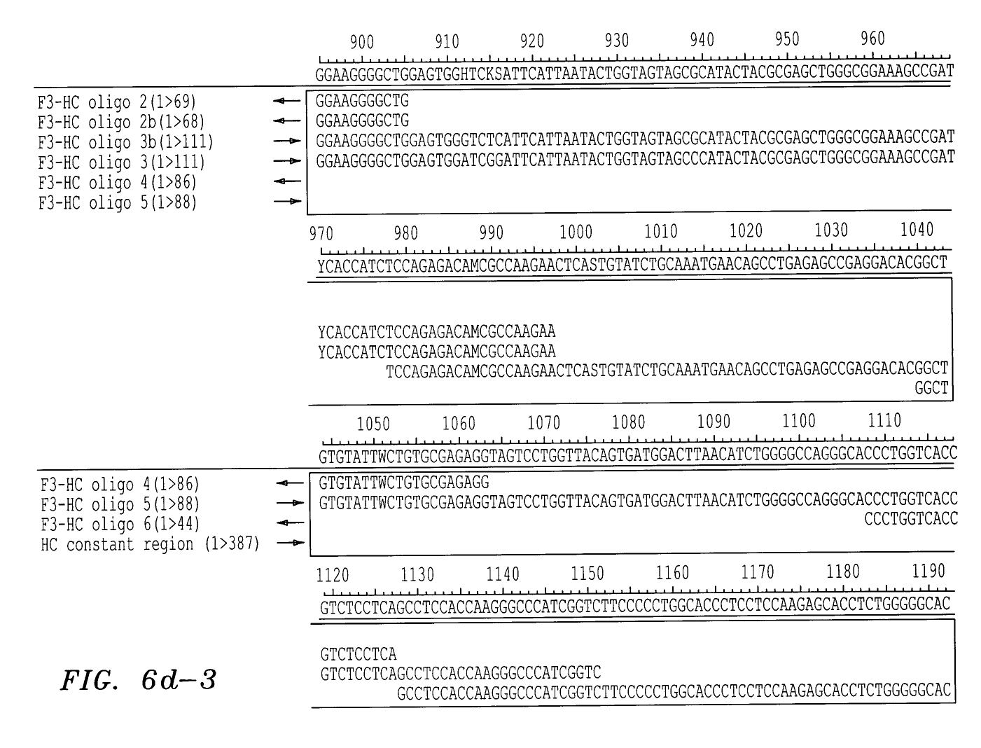

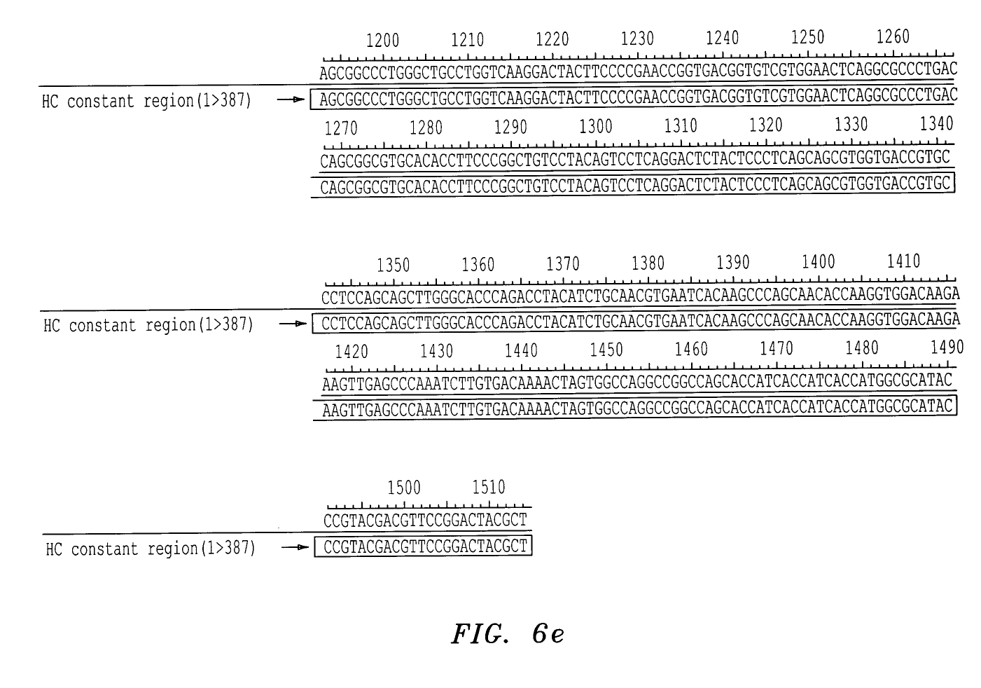

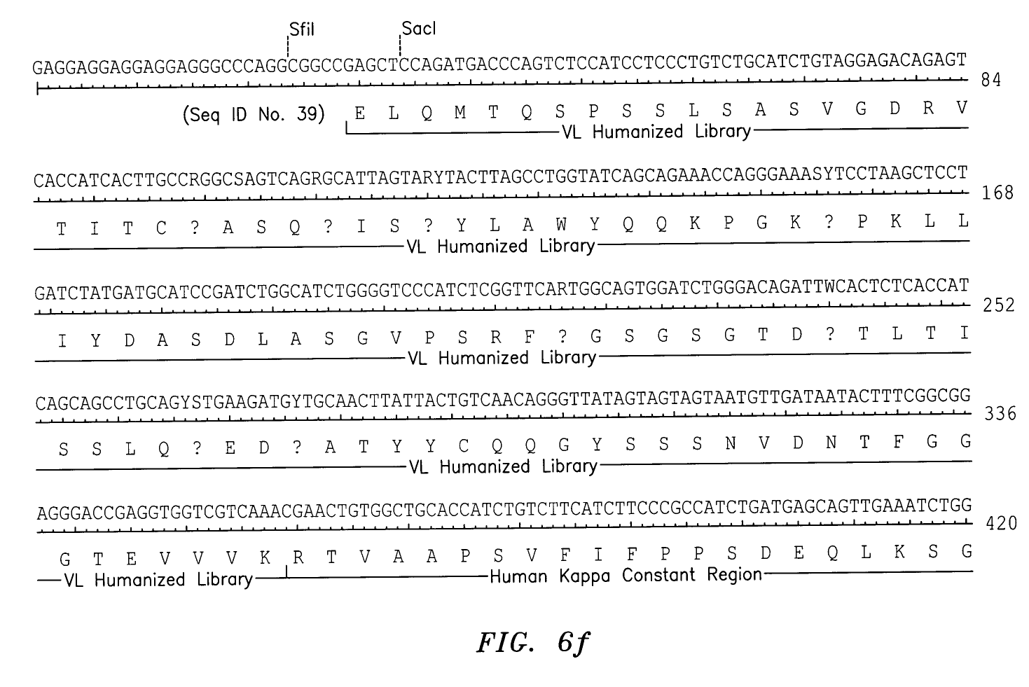

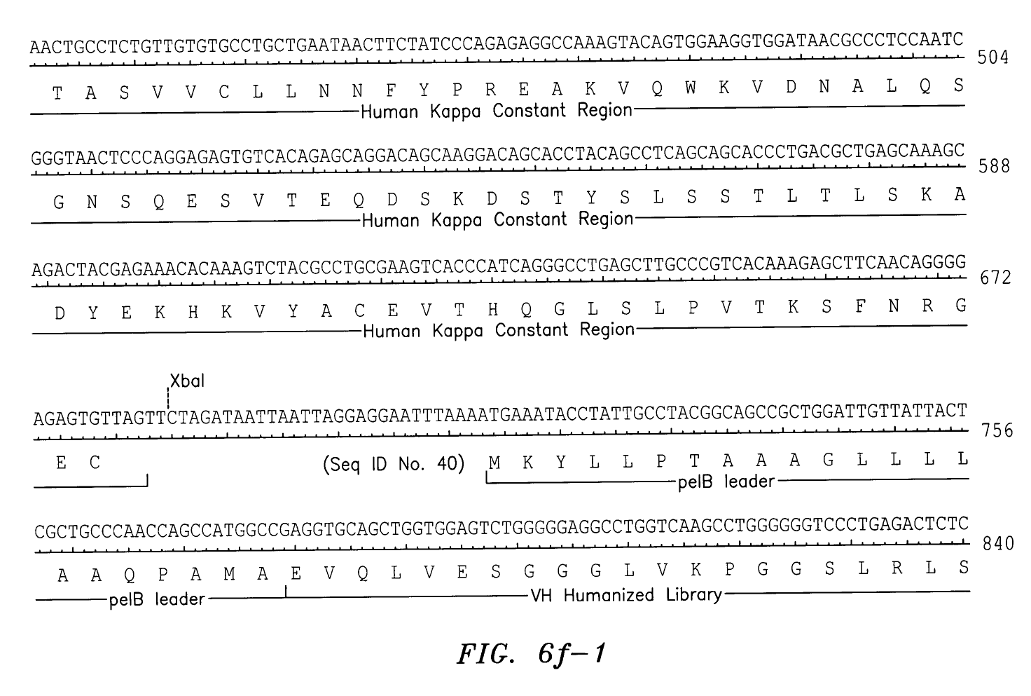

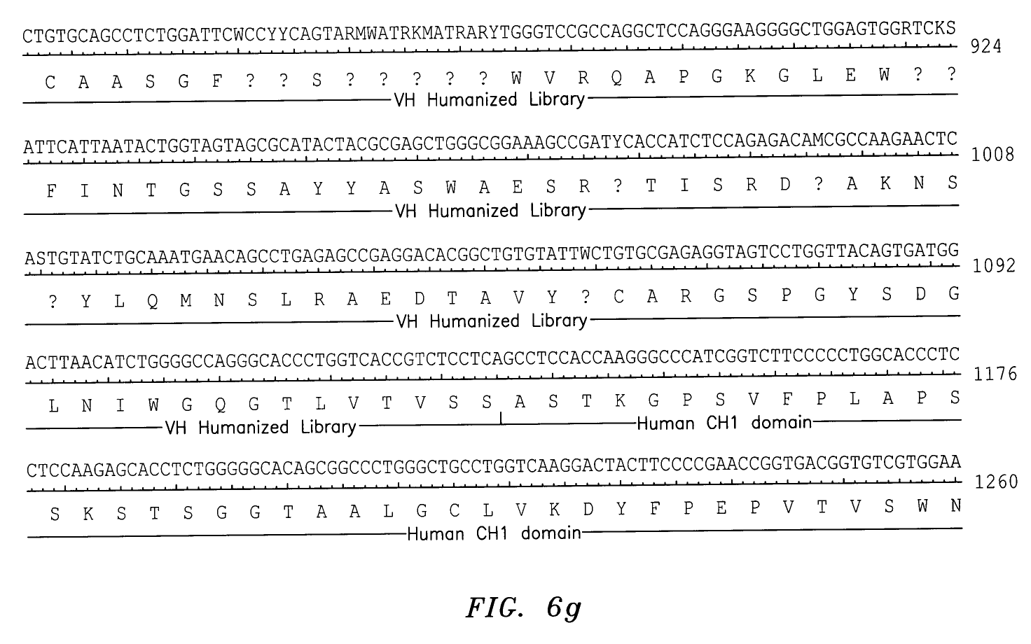

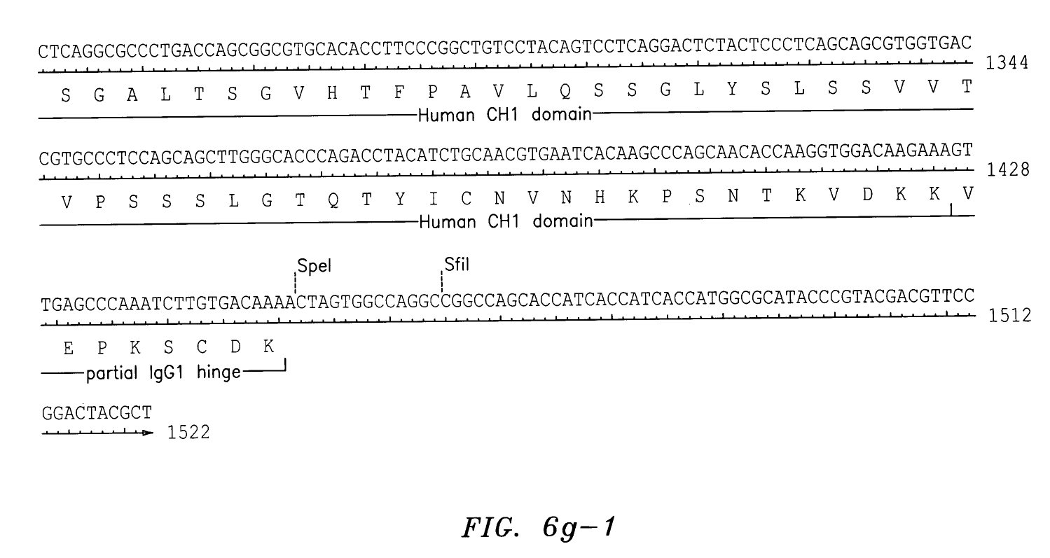

The VH and VL domains of the humanized F3 library were constructed by sequential PCR reactions. The PCR-assembled VH and VL sequences were fused to their respective fully human constant regions and then light and heavy chains were combined by overlap extension PCR into humanized F3 Fab inserts (FIGS. 6a-6g).

Step 1:

Oligonucleotides (60 pmol each) were combined as outlined in the table below for PCR overlap extension using the Expand High Fidelity PCR system (Roche Molecular Biochemicals, Indianapolis, Ind.).

| Light Chain | Light Chain |
|---|---|
| Product Generated | oligonucleotides |
| I | 1 + 2 |
| II | 1 + 2B |
| III | 3 + 4 |
| IV | 3 + 4B |
| V | 5 + 6 |
| Heavy Chain | Heavy Chain |
| Product Generated | oligonucleotides |
| I | 1 + 2 |
| II | 1 + 2B |
| III | 3 + 4 |
| IV | 3 + 4B |
| V | 5 + 6 |

The reaction mixture was heated to 94° C. and then underwent 15 rounds of thermocycling (94° C. for 15 seconds, 48° C. for 30 seconds and 72° C. for 45 seconds), followed by 2 minutes at 72° C. and a 4° C. hold. The reaction products were recovered using QIAquick PCR Purification Kit spin columns (Qiagen, Valencia, Calif.). DNA products were quantitated by OD260.

Step 2:

The PCR products from step 1 were combined according to the tables below for further assembly by PCR using the Expand High Fidelity PCR system (Roche Molecular Biochemicals, Indianapolis, Ind.). Each PCR product pair had regions of overlap necessary for annealing and overlap extension by the polymerase. The extended product could then be amplified by the flanking primer oligonucleotides added as detailed in the tables below. Note that PCR product #5 in step 2 is the same as PCR product V in step 1, no additional PCR amplification was necessary. The reaction mixture was heated to 94° C. and then underwent 15 rounds of thermocycling (94° C. for 15 seconds, 48° C. for 30 seconds and 72° C. for 45 seconds), followed by 2 minutes at 72° C. and a 4° C. hold. The reaction products were recovered by ethanol precipitation and run on a 1% LMP gel. Bands representing the desired amplification products were isolated and purified using QIAquick Gel Extraction Kit (Qiagen, Valencia, Calif.).

F3: Light Chain

| LC Product | F3 Step 1 LC | | |
|---|---|---|---|
| Generated | product | | F3 LC Primers |
| 1 | I | + III | Oligo 1, oligo 4 |
| 2 | I | + IV | Oligo 1, oligo 4B |
| 3 | II | + III | Oligo 1, oligo 4 |
| 4 | II | + IV | Oligo 1, oligo 4B |
| 5 | V | | (remaining only) |

F3: Heavy Chain

| HC Product | F3 Step 1 LC | | |
|---|---|---|---|
| Generated | product | | F3 LC Primers |
| 1 | I | + III | Oligo 1, oligo 4 |
| 2 | I | + IV | Oligo 1, oligo 4 |
| 3 | II | + III | Oligo 1, oligo 4 |
| 4 | II | + IV | Oligo 1, oligo 4 |
| 5 | V | | (remaining only) |

Step 3:

The PCR products from step 2 were combined according to the tables below for further assembly by PCR using the Expand High Fidelity PCR system (Roche Molecular Biochemicals, Indianapolis, Ind.) as described above in step two. These PCR products were the assembled VL and VH genes.

F3: Light Chain

| LC Product | Step 2 LC | |
|---|---|---|
| Generated | products | F3 LC Primers |
| VL a | 1 + 5 | Oligo 1, oligo 6 |
| VL b | 2 + 5 | Oligo 1, oligo 6 |
| VL c | 3 + 5 | Oligo 1, oligo 6 |
| VL d | 4 + 5 | Oligo 1, oligo 6 |

F3 Heavy Chain

| HC Product | F3 Step 1 LC | |
|---|---|---|
| Generated | product | F3 LC Primers |
| VH a | 1 + 5 | Oligo 1, oligo 6 |
| VH b | 2 + 5 | Oligo 1, oligo 6 |
| VH c | 3 + 5 | Oligo 1, oligo 6 |
| VH d | 4 + 5 | Oligo 1, oligo 6 |

Step 4:

Fragments encoding kappa constant region or heavy chain CH1 domain were generated by PCR amplification of pRL4-TT, a vector which contained anti-tetanus toxoid kappa light chain and Fd heavy chain genes. The primers used for the light chain constant region amplification allowed the incorporation of vector based control elements for the heavy chain gene at the 3′ end of the LC constant region. Primers used for LC constant region amplification were HkC-F 5′ CGGACTGTGGCTGCACCATCTGTC 3′ (Seq. Id. No. 2) and Lead B 5′ GGCCATGGCTGGTTGGGCAGC 3′ (Seq. Id. No. 3). The primers used for heavy chain CH1 amplification allowed the incorporation of vector based elements at the 3′ end of the CH1 domain such as restriction sites necessary for later cloning steps. Primers used for HC CH1constant region amplification were HlgGCH1-F 5′ GCCTCCACCMGGGCCCATCGGTC 3′ (Seq. Id. No. 4) and N-dp 5′ AGCGTAGTCCGGAACGTCGTACGG 3′ (Seq. Id. No. 5). Constant region sequences were amplified using the Expand High Fidelity PCR system (Roche Molecular Biochemicals, Indianapolis, Ind.). PCR reaction mixtures containing pRL4TT, dNTP, reaction buffer and respective primer sets (HKC-F and lead B for the light chain; HlgCH1-F and N-dp for the heavy chain) were prepared. The reaction mixture was heated to 94° C. and then underwent 15 rounds of thermocycling (94° C. for 15 seconds, 48° C. for 30 seconds and 72° C. for 90 seconds), followed by 2 minutes at 72° C. and a 4° C. hold. The reaction products were recovered by ethanol precipitation and run on a 1% LMP gel. Bands representing the desired amplification products were isolated and purified using QIAquick Gel Extraction Kit (Qiagen, Valencia, Calif.).

Step 5:

The VL products from step 3 (VL a-d) were combined with the kappa constant region (Ck) product generated in step 4 by overlap extension PCR to generate full length light chains. The VL and Ck fused due to complementary sequences located at the 3′ ends of the VL genes (sense strand) and the constant region PCR product (anti-sense strand). PCR reactions contained VL and CK DNA, primers RSC-F 5′ gag gag gag gag gag gag gcg ggg ccc agg cgg ccg agc tc 3′ (Seq. Id. No. 6) and lead B, dNTPs, and Expand High Fidelity PCR system's reaction buffer and enzyme (Roche Molecular Systems). The reaction was heated to 94° C. and then underwent 15 rounds of thermocycling (94° C. for 15 seconds, 48° C. for 30 seconds and 72° C. for 90 seconds), followed by 2 minutes at 72° C. and a 4° C. hold. The reaction products were recovered by ethanol precipitation and run on a 1% LMP gel. Bands representing the desired amplification products were isolated and purified using QIAquick Gel Extraction Kit (Qiagen, Valencia, Calif.).

Using the same technique described above for light chain assembly, VH products from step 3 (VH a-d) were fused with the CH1 PCR product from step 4 to generate Fd heavy chain fragments. Primers used for the amplification of the Fd heavy chain products were leadVH 5′ GCT GCC CM CCA GCC ATG GCC 3′ (Seq. Id. No. 7) and N-dp.

Step 6:

Light chain and heavy chain Fd products generated in step 5 were combined by overlap extension PCR to generate an Fab insert containing an Sfi I restriction site, light chain gene, a ribosomal binding site and pel B leader in frame with the heavy chain Fd gene which follows, a second Sfi I restriction site, and His 6 and HA epitope tags. The light chain constant region was PCR derived from a vector in such a way as to include the pel B leader. The heavy chain Fd products also contain a portion of the pel B leader so that when combined with the light chain products, they were able to anneal at this region and be extended by the polymerase to generate the full Fab insert. That insert was then further amplified using the outer primers RSC-F and N-dp.

The humanized F3 Fab products were gel purified and recovered as described above followed by digestion with Sfi I restriction enzyme. The digested fragments were then gel purified again prior to being cloned into Sfi I digested chloramphenicol-resistant pRL4-ss vector.

Selection of Humanized F3 Display Library

The phagemid library displaying the humanized F3 Fabs was selected by panning four rounds against immobilized human PDGF-BB antigen (R+D Systems, MN). 500 ng PDGF-BB/well was used to coat the microtiter well in the first and second rounds followed by 250 ng in the third and fourth rounds. The library was applied to the PDGF-BB coated wells and allowed to bind for two hours at 37°. Non-specific binders were washed away using three 0.5% TBS/Tween washes in the first round, 5 washes in the second round and 10 washes in the last 2 rounds. Displayed Fabs that remained on the antigen coated wells were eluted using 0.1 M HCl pH 2.2 containing 1 mg/ml BSA followed by neutralization with 2 M Tris. Eluted Fab-bearing phagemid particles were mixed with fresh ER2537 bacterial cells after each round of panning to amplify the enriched pool of phagemid particles for the next round of panning.

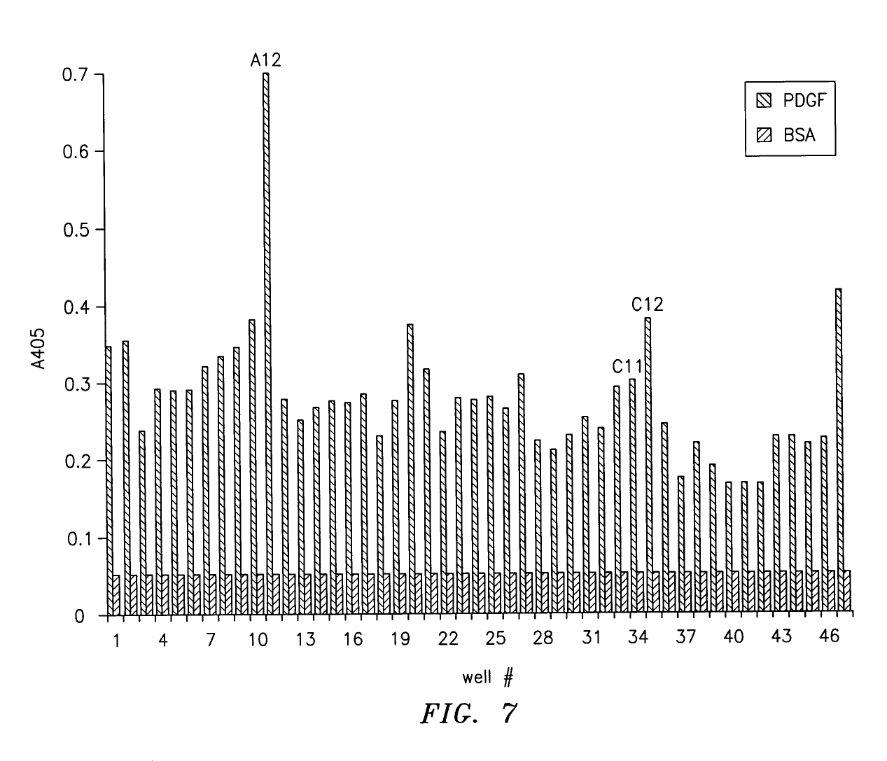

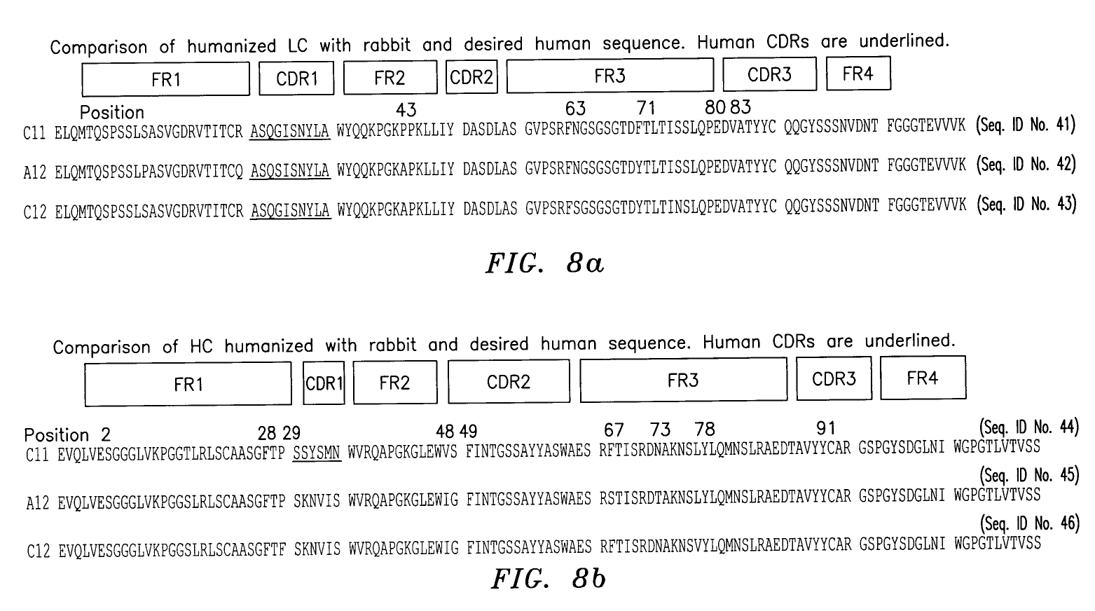

After the fourth round of panning, the ability of humanized F3 antibodies to bind PDGF-BB antigen was assessed by ELISA. High protein binding 96-well plates (Costar, NY) were coated overnight with 100 ng of recombinant PDGF-BB (R+D Systems, MN) in 0.1 M NaHCO3 pH 8.6 at 4° C. The plates were blocked with 1% BSA for 1 hour at 37° C. followed by 3 washes of PBS/0.05% Tween solution. E. coli culture supernatants or purified antibody was added at various concentrations in PBS/1% BSA for 2 hours at 37° C. on a shaker. After 3 washes with PBS/0.05% Tween, anti-HA primary antibody (12CA5 ascites) at a 1:10,000 dilution in PBS/1% BSA was added and incubated for 2 hours. Wells were washed 3× with PBS/0.05% Tween followed by incubation with alkaline phosphatase-conjugated anti-mouse IgG (Sigma, St. Louis, Mo.) for 2 hours. After incubation, the plates were washed 3× with PBS/0.05% Tween and 3× with PBS. Color development after addition of Sigma 104 substrate in PNPP buffer (10 mM diethanolamine, (0.5 mM MgCL2, pH 9.5) was determined at OD 405 using a microplate reader (Molecular Devices). See FIG. 7. Three humanized Fabs (C11, C12, and A12) which demonstrated significantly higher binding to PDGF-BB compared to BSA were further characterized. Sequence analysis of the clones revealed that diversification of the CDR 1 was successfully achieved. Clone C11 retained human CDR 1 in both light and heavy chains. Clones A12 and C12 both selected human CDRs in the light chain CDR1 position while rabbit CDR was selected by both clones in the heavy chain CDR1 position (FIGS. 8a and 8b).

III. PDGF-Receptor Competition Assays

Antibody Purification

Humanized F3 Fabs (C11, C12, and A12) containing a His 6 purification tag were purified from periplasmic preparations of transfected bacterial cultures using Ni-column chromatography as described above for the purification of scFv F3. Purified Fab was dialyzed against PBS buffer.

Competition ELISA

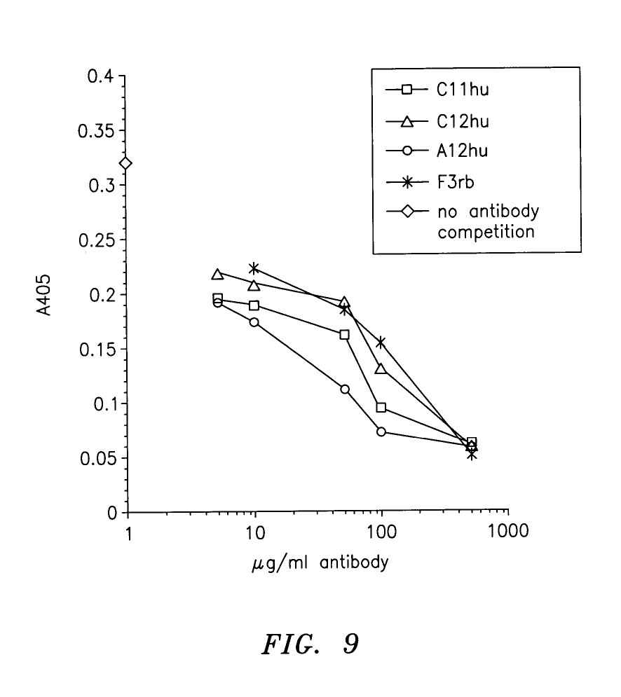

The ability of the humanized F3 Fabs (C11, C12, and A12) to compete with the PDGF-bb receptor for binding to the PDGF-BB cytokine was determined in a competition ELISA assay as described above. Results from the competition assay revealed that all three humanized F3 Fabs (C11, C12, and A12), as well as the parental scFv F3, inhibited PDGF-BB binding to its target receptors (FIG. 9). These results indicate that the humanized Fabs have affinities similar to the rabbit F3 scFv.

Luciferase assay

The ability of humanized Fabs C11, C12, and A12 to functionally block the interaction between PDGF-BB and its receptor in a cell based assay is determined using the Luciferase assay. NIH3T3 cells express murine PDGF receptors that can bind to human PDGF-BB cytokine. In order to determine the extent of the PDGF-b receptor activation, the NIH3T3 cells are transfected with a fos promoter/luciferase reporter gene construct. On binding to the cytokine, the receptor becomes activated and induces a cascade of signals which ultimately activate the Fos promoter that is linked to a luciferase reporter gene. Luciferase activity, which is correlated to the amount of luciferase expressed, is quantitated using a luminometer.

NIH 3T3 cells are plated at 3×10⁵ cells per 6 cm dish and transfected on the following day. Transfection is performed with the Effectine lipofection reagent (Qiagen, Valencia, Calif.) using 0.1 ug of pEGFP (a tracer to measure transfection efficiency) and 0.2 ug of the Fos promoter/luciferase construct per plate. Transfected NIH3T3 cells are placed in 0.5% serum 24 hours following transfection and incubated for an additional 24 hours to reduce the background activation of the Fos promoter. Next, Fab are added to the cells along with PDGF-BB for 6 hours. Cells are harvested and then luciferase activity is assayed using 50 ug of cell lysate.

### EXAMPLE 2

### Preparation of a Murine Anti-PDGF Antibody

Immunization of Mice

2 Balb/c mice were immunized with recombinant PDGF-BB. First, they were administered 15 mg PDGF i.p. in complete Freund's adjuvant (Sigma). 2 weeks later they received i.p. the same amount of PDGF in incomplete Freund's adjuvant followed by a similar injection 3 weeks later. After another 3 weeks, the mice received an i.v. boost with 15 mg PDGF-BB in PBS. Three days later, the serum anti-PDGF antibody titer reached 1:20,000 as determined by solid phase ELISA. Mice were sacrificed and spleen and bone marrow was collected to isolate RNA with Tri reagent (Molecular Research Center, Cincinnati, Ohio). First-strand cDNA was synthesized using “Superscript Preamplification System for first strand cDNA synthesis kit” with oligo (dT) priming (GibcoBRL).

Mouse Antibody Library

Mouse light chain and heavy chain fd regions were amplified from the first strand cDNA by 20 cycles of PCR using heavy and light chain primers as described in the Barbas III, et al. (2001) Phage Display, A Laboratory Manual, Cold Spring Harbor Laboratory Press, Cold Spring Harbor, N.Y. pp. 9.54-9.57. Additional primers are MCKappaB: 5′ GAGGAGGAGGAGGAGTCTAGAATTAACACTCATTCCTGTTGAA 3′ (Seq. Id. No. 8) and MIGGIB: 5′ GAGGAGGAGGAGGAGACTAGTACAACTCCTGGGCACAAT 3′ (Seq. Id. No. 9). Light chain DNA was cloned into phagemid vector pRL4 using Sac1 and Xba1 and heavy chain DNA was cloned using Spe1 and Xho1. Transformation into ER cells yielded a library of 6×10⁷ independent clones. The library was panned 4 times against immobilized recombinant human PDGF-BB starting with 500 ng protein/well in the first and second pan, 250 ng in the third and fourth pan. The bound phage was washed 3 times with 0.5% Tween in TBS in the first round, 5 times in the second round and 10 times in the last 2 rounds. Phage was eluted using 0.1 M HCl pH 2.2 containing 1 mg/ml BSA, then it was neutralized with 2 M Tris. Clones obtained after round 4 were tested for binding to PDGF-BB in solid phase ELISA. 96-well high protein binding plates (Costar, NY) were coated with 100 ng recombinant PDGF-BB (R+D Systems, MN) in 0.1 M NaHCO3 pH 8.6 overnight at 4° C. The plate was blocked with 1% BSA for 1 h at 37° C. followed by 3 washes with PBS/0.05% Tween. E. coli culture supernatants or purified antibody was added at various concentrations in PBS/1% BSA for 2 h at 37° C. on a shaker. After 3 washes with PBS/0.05% Tween, anti-HACA5 (ascites) at a 1:10,000 dilution in PBS/1% BSA was added for 2 h. Incubation with alkaline phosphatase-conjugated anti-mouse IgG (Sigma) for 2 h was followed by 3 washes with PBS/0.05% Tween and 3 washes with PBS. Color development after addition of Sigma 104 substrate in PNPP buffer (10 mM diethanolamine, (0.5 mM MgCL2, pH 9.5) was determined at OD 405 using a microplate reader (Molecular Devices).

Panning the murine PDGF library on PDGF resulted in a large number of very similar clones. Clones were categorized into 2 families based on differences in light chain CDR1 and CDR2 and heavy chain CDR2. Several residues distinguishing between the families were identified and are presented in the table below.

| | Kabat | | | |
|---|---|---|---|---|
| Chain | position # | general location | C1-type family | AK-type family |
| light | 31 | CDR1 | serine | argenine |
| light | 51 | CDR2 | alanine | threonine |
| heavy | 57 | CDR2 | threonine | alanine |

Angiogenesis Assay

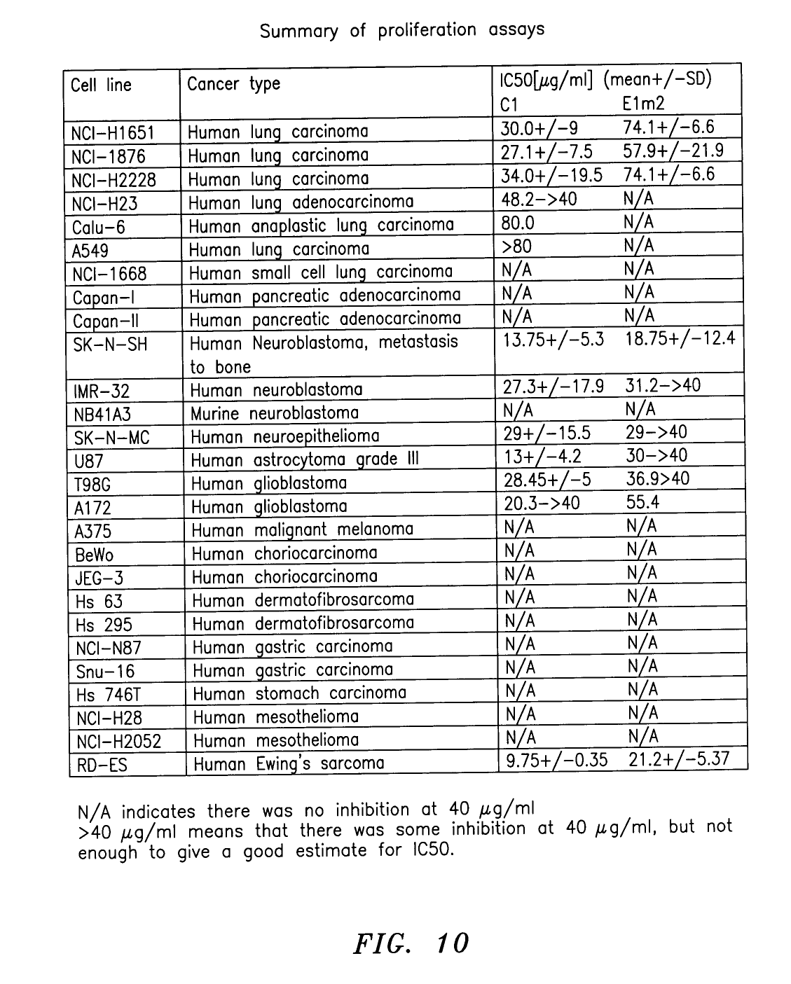

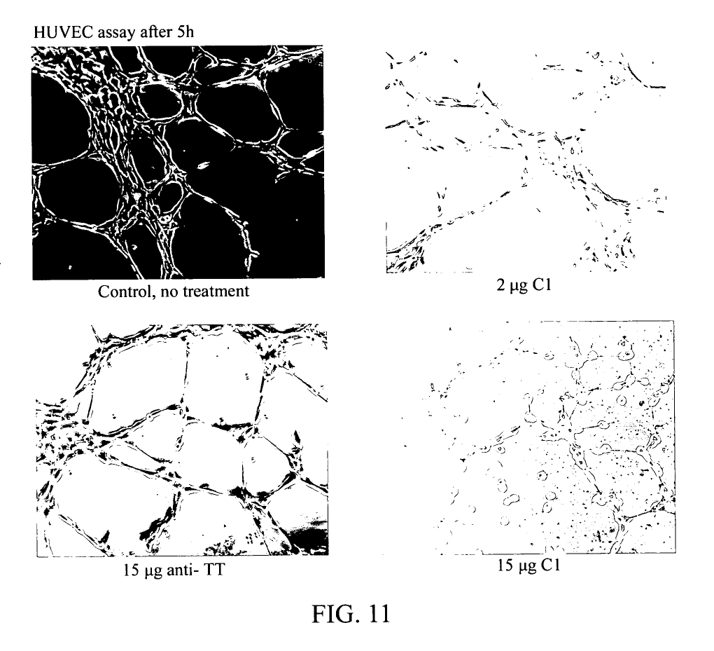

To determine whether the anti-PDGF antibody C1 affects blood vessel formation, HUVEC assays were performed. Wells of 24 well plates were coated with 500 ul matrigel each for 30 min at 37° C. Forty thousand HUVEC cells were added per well with or without the addition of anti-PDGF antibody Cl or control antibody TT. Cells were photographed after 5 h and 24 h and tube formation was observed. Results after 5 h are shown in FIG. 11.

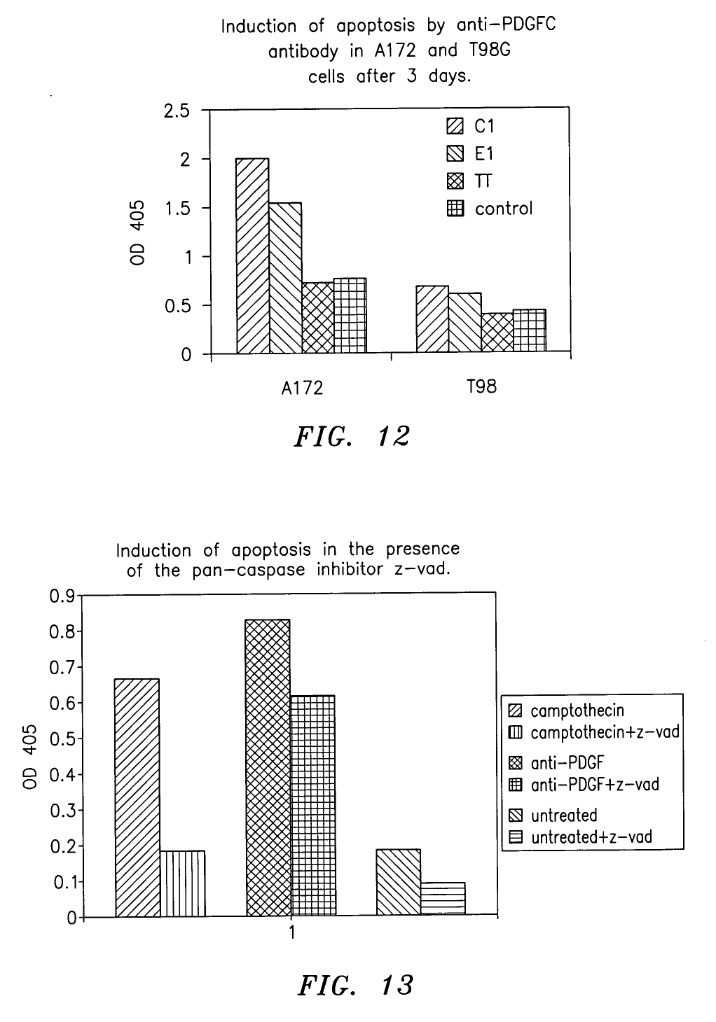

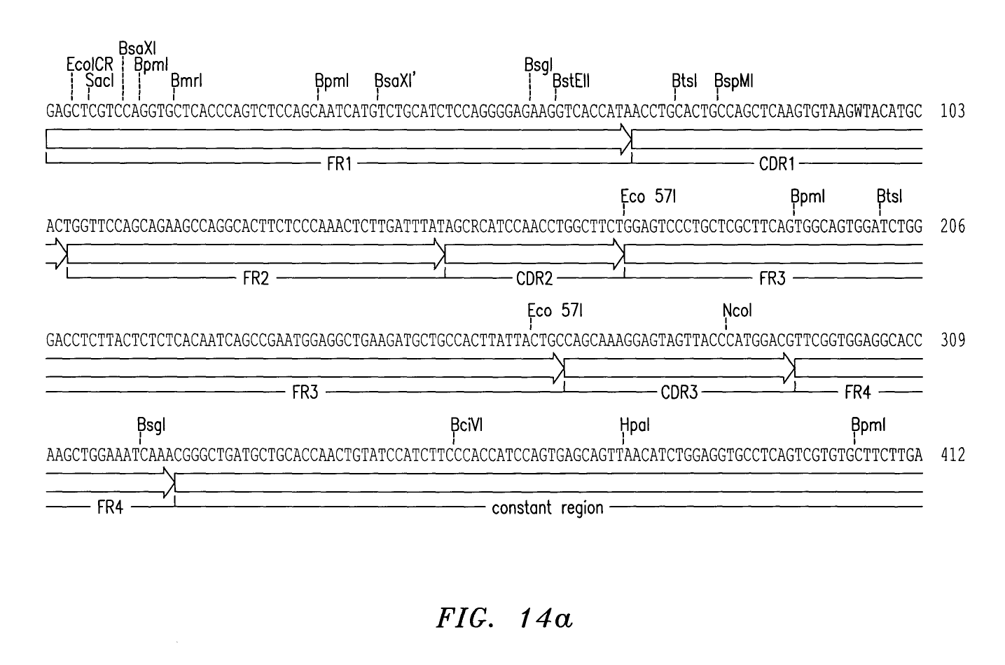

Clone C1 (of the C1-type family) was selected from the library and used in the humanization procedures which follow. The sequences of C1 mouse anti-PDGF Fab light and heavy chains are shown in FIGS. 14a and 14b, respectively. Antibodies from the AK-type family also contributed to the humanization design. The AK-type family's amino acids in positions #31 and #51 for the light chain and #57 for the heavy chain were included as choices during the humanization process (denoted as an X in the “synthetic C1” sequence in FIGS. 15a and 15b).

### EXAMPLE 3

### Humanization

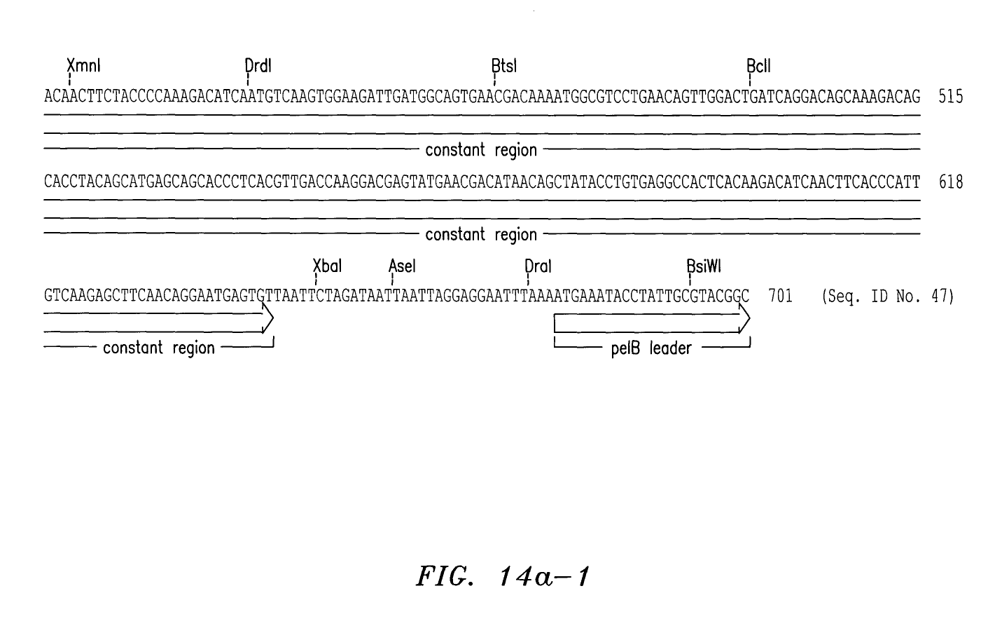

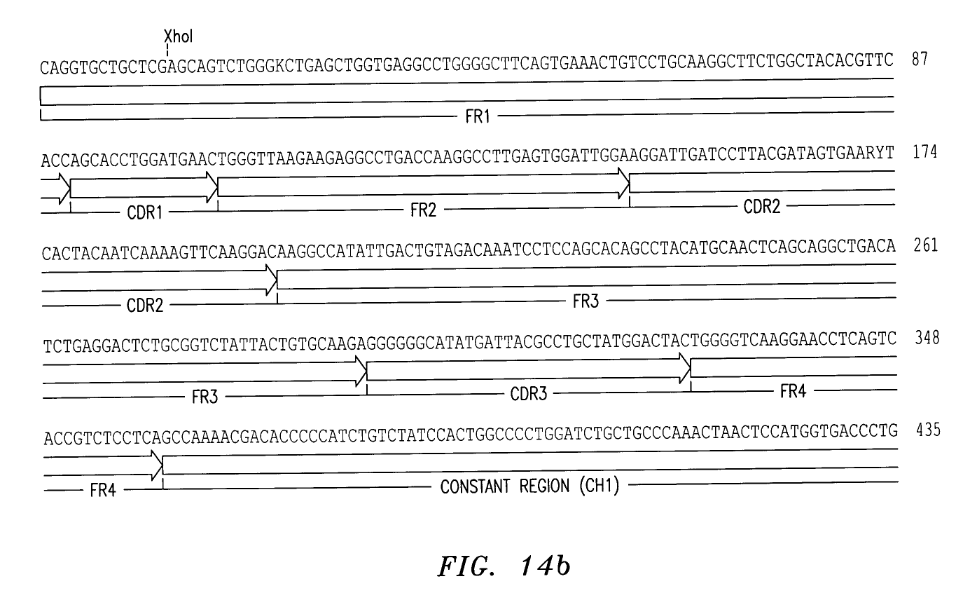

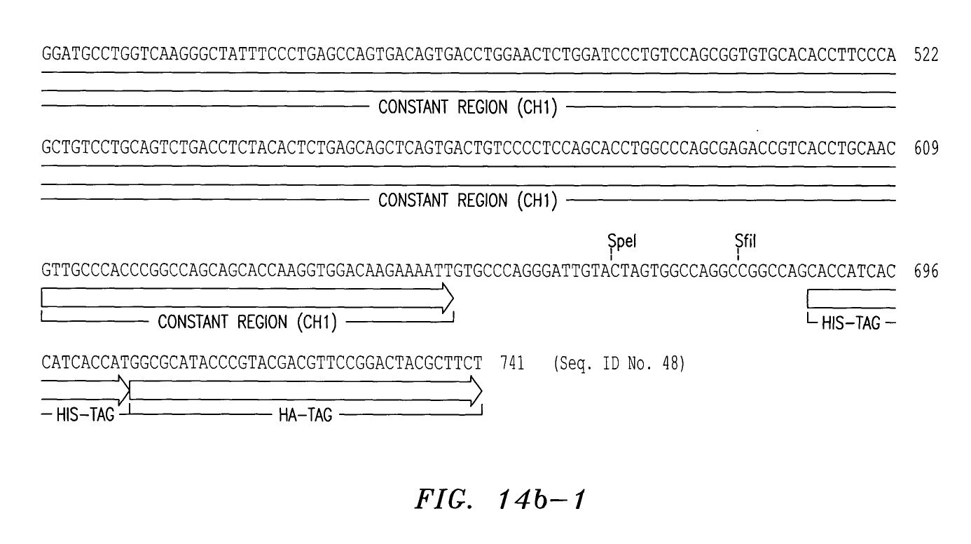

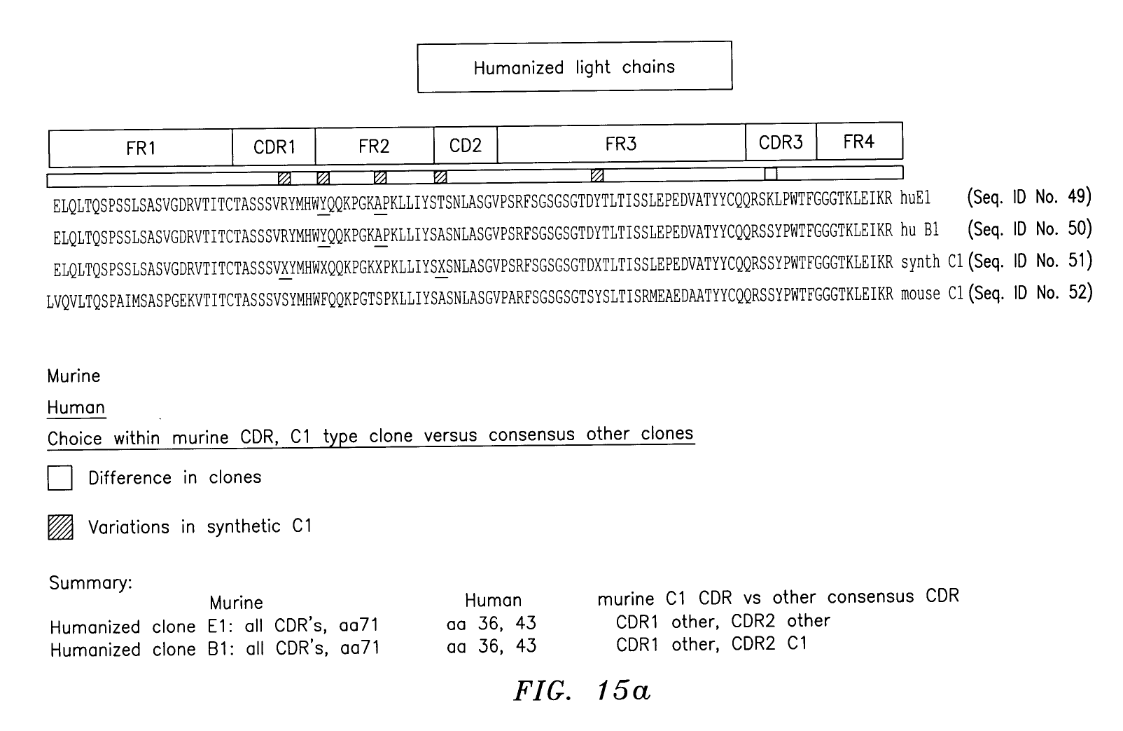

Human germ-line VL and VH sequences with the highest degree of homology to the corresponding mouse sequences were identified from the Vbase directory of human V gene sequences (http//www.mrc-cpe.cam.ac.uk/imt-doc) by nucleotide sequence alignment. Human FR4 sequences to be used for the humanization were identified based on amino acid homology of C1 sequence with the Kabat database of immunoglobulins located at http://www.immuno.bme.nwu.edu. Residues known to be important for maintaining the epitope conformation (Foote J, Winter G. J Mol Biol Mar. 20, 1992; 224(2): 487-499) were diversified to allow the usage of either the mouse or the human sequence while avoiding mouse residues in positions known to be surface-exposed (Pedersen et al, J. Mol. Biol. 1994, 235, 959-973 and Santos and Padlan, Progress in Nucleic Acid Research and Mol. Biology 60, 169-194). Also, CDR1 and CDR2 were diversified for mouse or human usage, whereas the mouse CDR3 was grafted. Overlapping oligonucleotides were synthesized as outlined in FIGS. 16a and 16b and assembled in sequential overlapping PCR reactions with 15 cycles each using the Expand High Fidelity PCR system (Roche Molecular Systems). See FIGS. 15a and 15b. The synthetic VL and VH coding sequences were fused to human Ck and CH1 coding sequences obtained by PCR of tetanus toxoid antibody cloned in pRL4. The final constructs were cloned into pRL4 containing a chloramphenicol—instead of carbecillin—resistant gene using Sac1/Xba1 for the light chain and Xho1/Spe1 for the heavy chain. The resulting library of 1×10⁶ independent clones was panned on PDGF-BB as described for the mouse library.

Antibody Purification

Antibody was purified from periplasmic preparations of transfected bacterial cultures followed by passing through a Ni-column. Periplasmic fractions were obtained by adding 20% sucrose in 30 mM Tris pH 8 to the bacterial pellets for 20 min. The suspension was spun down for 10 min at 9000 g in 4° C. The supernatant was kept on ice and the pellet was resuspended in sterile cold water and left on ice for 10 min. After a 10,000 g spin, the supernatant was combined with the sucrose supernatant and spun at 12,000 g for 20 min. 1 tablet of Roche Mini protease inhibitor cocktail without EDTA and NaCl to 0.2 M and imidazole to 10 mM was added. After filtration, the periplasmic fraction was loaded into a superloop and then run over a Ni-charged 5 ml HiTrap chelating column (Pharmacia) with a flowrate of 2 ml/min using an AktafPLC machine (Pharmacia). Washing buffer consisted of 20 mM NaH2PO4/Na2HPO4, 0.5 M NaCl and 10 mM imidazole at pH 7.4 and elution buffer was made of 20 mM NaH2PO4/Na2HPO4 0.5 M NaCl and 500 mM imidazole at pH 7.4. 1 ml fractions were collected and those exhibiting elevated OD 280 values were pooled and dialyzed against PBS. For cell based assays, endotoxin was removed from dialyzed fractions of purified antibody by running over a pyrogen free 4 ml Affi-Prep Polymyxin Matrix (Bio Rad) drip column at a flow rate of 1 ml/min using PBS as an equilibration buffer. Endotoxin levels were quantified using the LAL method (Bio Whittaker QCL-1000), and the sample rerun over the Polymyxin resin when additional endotoxin removal was necessary. An alternative method was to pass sample through a Q15 Strongly Ionic Anion Exchanger (Sartorius) at a 1 ml/min flow rate, using 50 ml 20 mM phosphate, 150 mM NaCl, pH 7.2 as an equilibration buffer.

### EXAMPLE 4

### Reactivity of Anti-PDGF Antibody

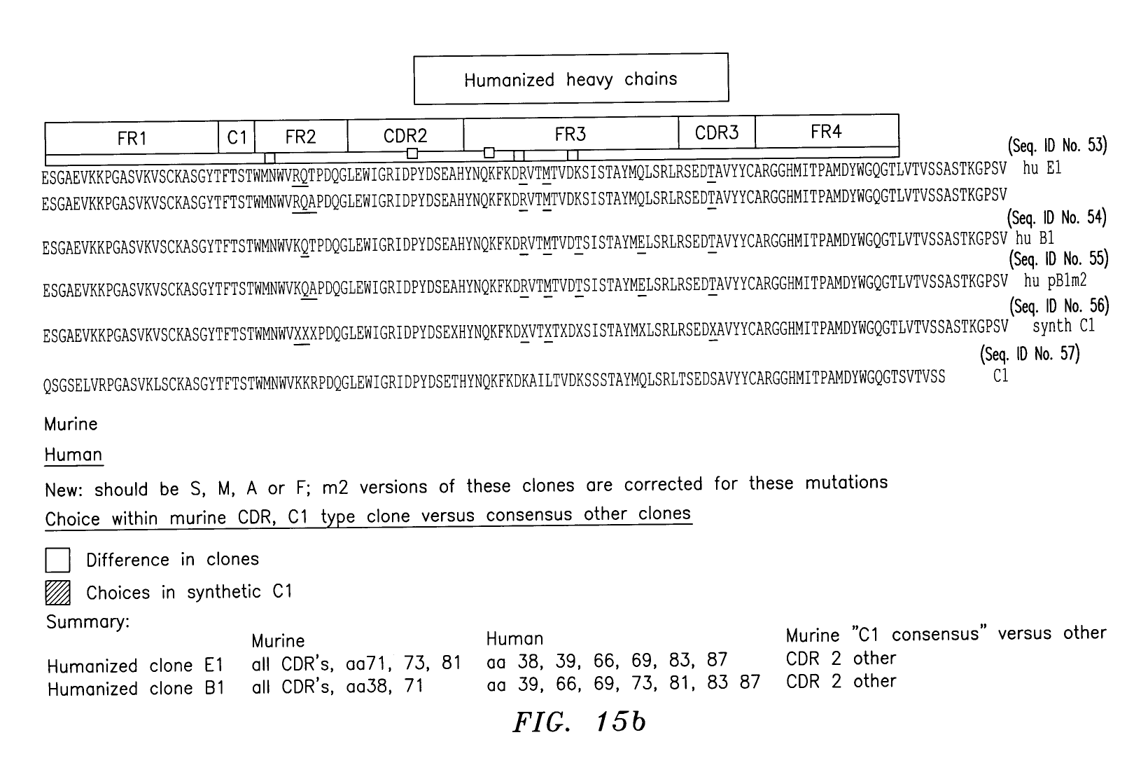

The binding of C1 anti-PDGF antibody was tested by solid phase ELISA, as described above, against various sources of PDGF. The results are set forth in FIG. 17.

C1 anti-PDGF antibody reacted strongly with human and porcine PDGF-BB, but not with rat PDGF BB. Reaction with human PDGF M was weak.

### EXAMPLE 5

### PDGF-Beta Receptor Competition Assay

96-well high protein binding plates were coated with 10 ng/well of recombinant human PDGF receptor beta/Fc chimera (R+D Systems) in PBS overnight at 4° C. The plate was blocked with 5% sucrose/1% BSA/PBS for 1 h at 37° C. Serial dilutions of purified anti-PDGF-BB antibody were incubated with 10 ng/ml recombinant human PDGF-BB at room temperature for 30 min before being added to the blocked and washed plate. After 2 h at room temperature, the plate was washed with PBS/0.05% Tween followed by incubation with biotinylated anti-PDGF-BB antibody (R+D Systems) for 2 h at room temperature. Streptavidin-alkaline phosphatase (Pierce) was added for 30 min at room temperature. After 3 washes with PBS/0.05% Tween and 3 washes with PBS, Sigma 104 substrate in PNPP buffer was added and the signal at OD405 was determined.

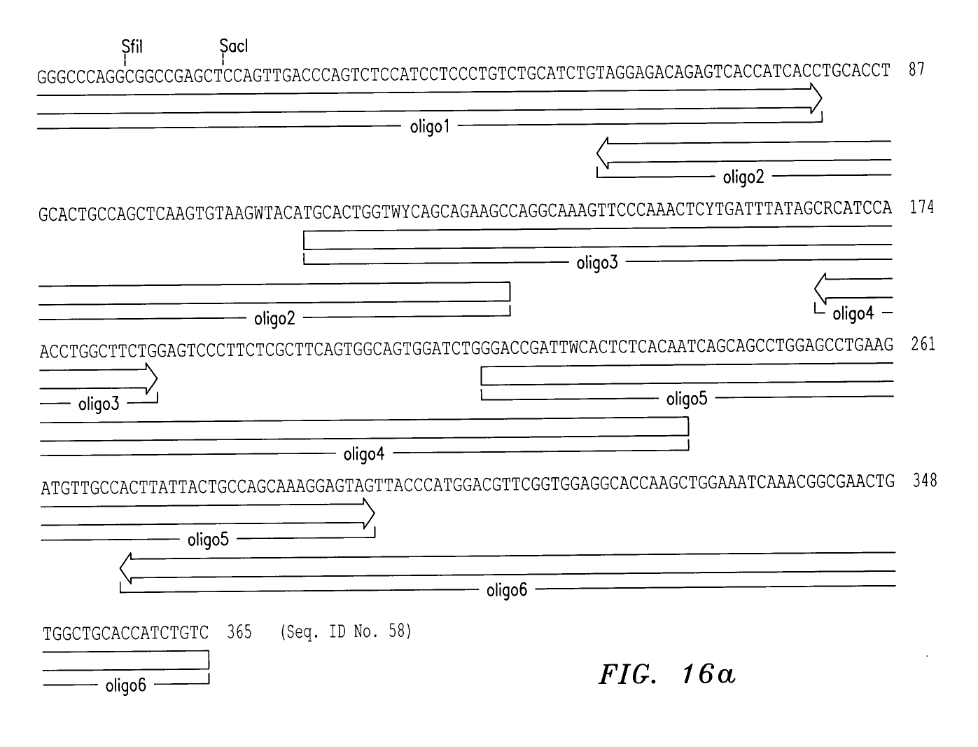

FIG. 18 shows the results obtained for the murine antibody C1, in comparison with a rabbit anti-PDGF antibody (A8).

### EXAMPLE 6

### Competition with PDGF-BB

Antibody C1 was tested for the ability to compete with PDGF BB in a signal transduction pathway. A luciferase assay was used as a readout.

For measurement of competitiveness, Fab containing bacterial supernatants (2 mls) mixed with PDGF-BB were applied to NIH3T3 cells which had been co-transfected with the Fos promoter/luciferase reporter construct. Co-transfections of 3T3 cells were performed by plating NIH 3T3 cells at 3×10⁵ cells per 6 cm dish and then transfecting the following day. NIH 3T3 cells were transfected using the Effectine lipofection reagent (Qiagen), transfecting each plate with 0.1 μm pEGFP (a tracer to measure transfection efficiency) and 0.9 μg of the Fos promoter/luciferase construct. 3T3 cells were placed in 0.5% serum 24 hours post transfection and incubated for an additional 24 hours in this low serum media to reduce the background activation of the Fos promoter. Antibody supernatants were then applied to these cells for 6 hours. Cells were harvested and luciferase assays performed using 50 μg of cell lysate.

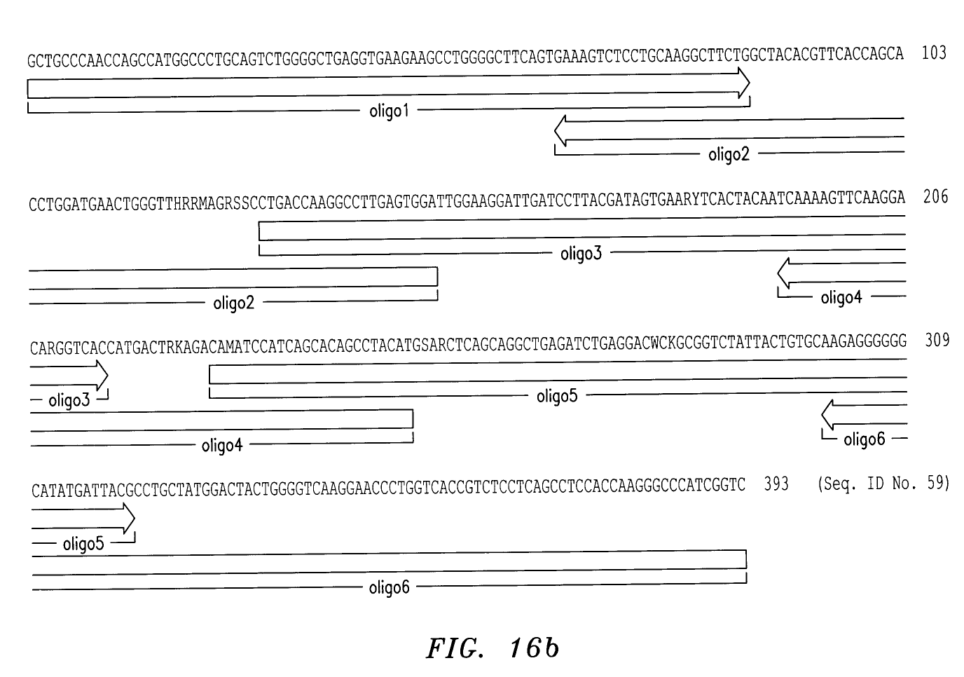

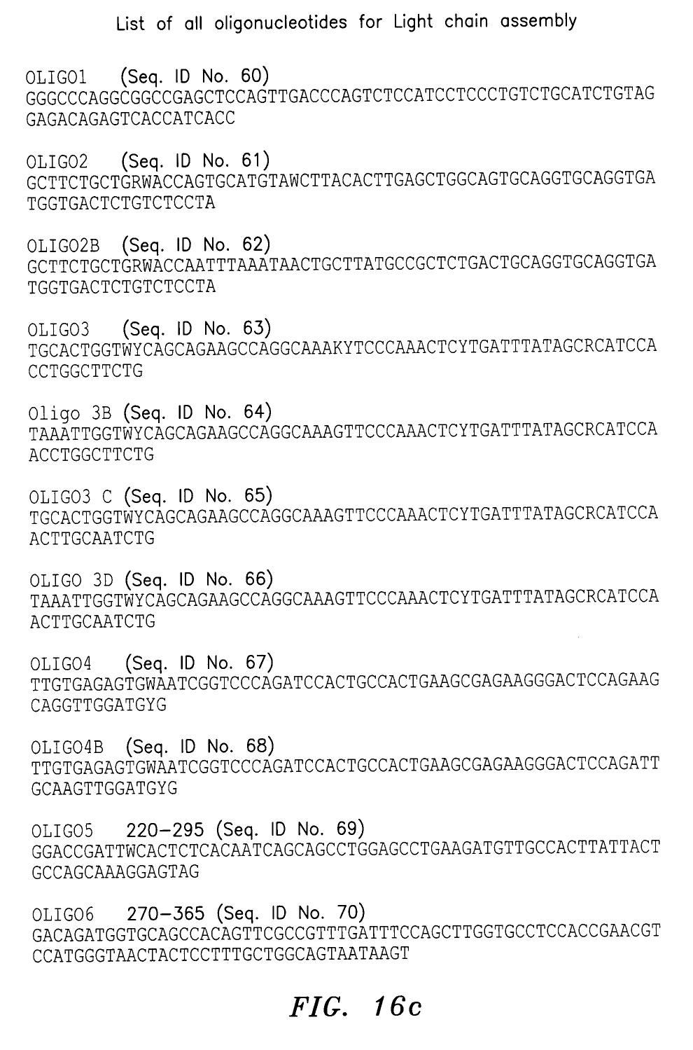

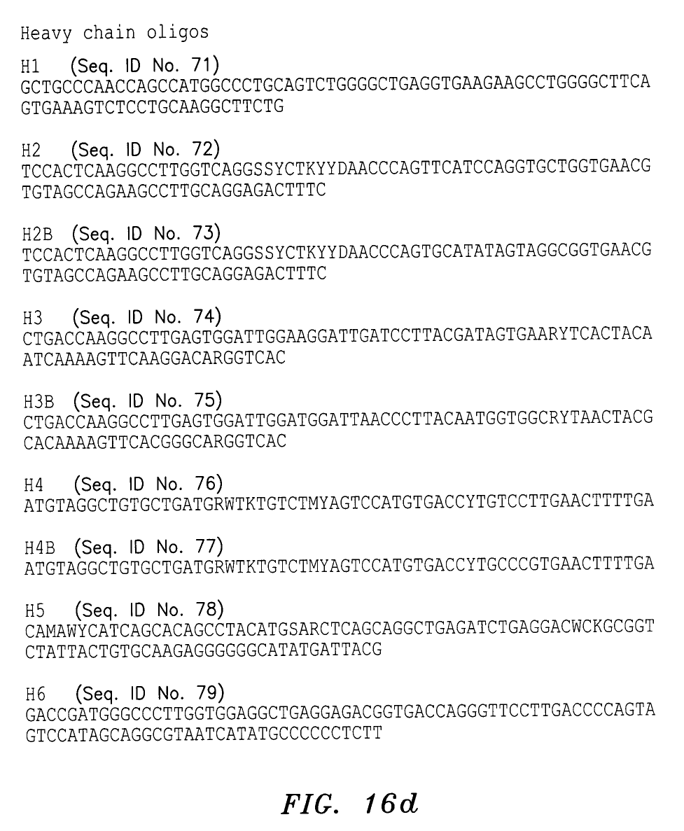

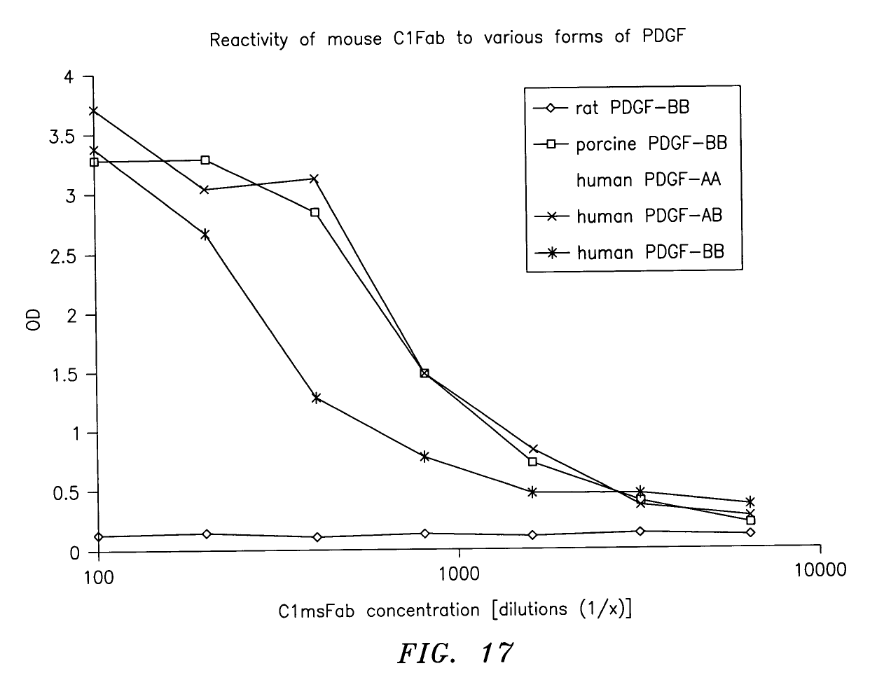

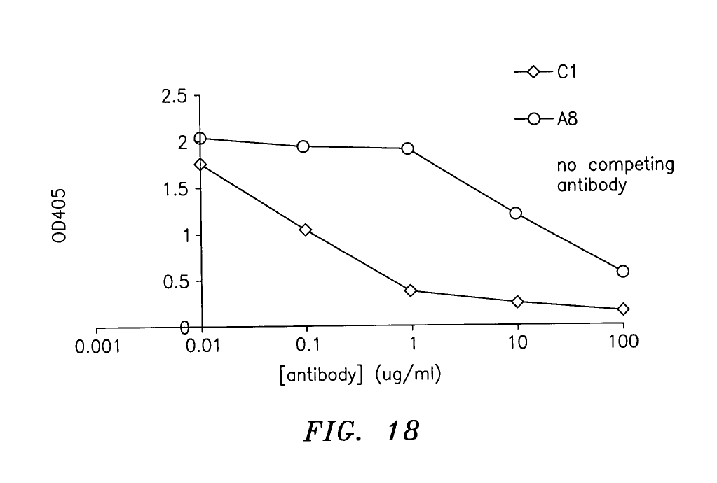

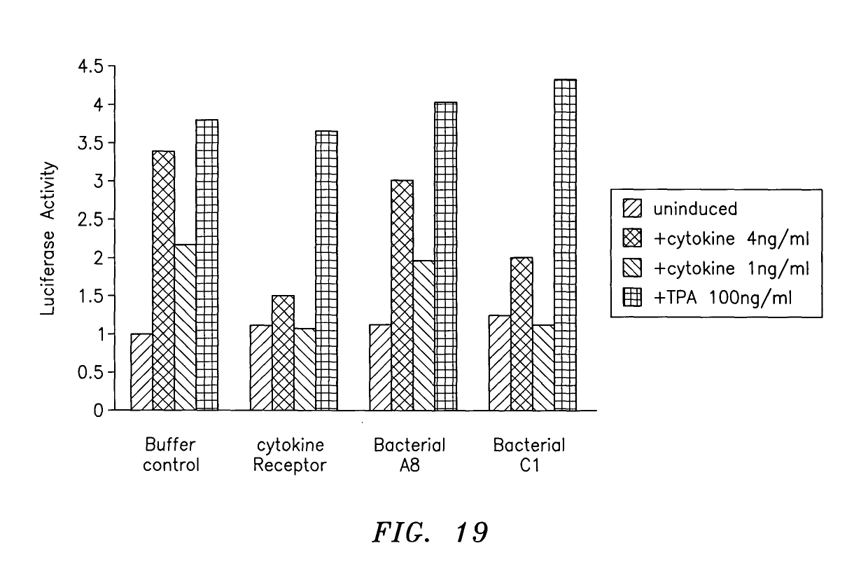

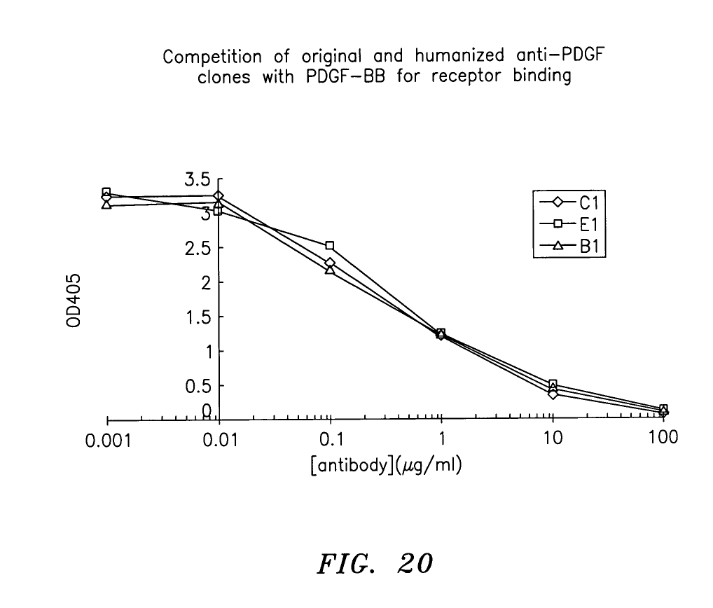

The results are shown in FIG. 19. C1 is able to inhibit signal transduction by PDGF-BB, but not tPA.

### EXAMPLE 7

### BIACore Affinity Measurements

BIAcore analysis of the original and humanized anti-PDGF clones was performed according to standard procedures. The cytokine was immobilized on the BIAchip by chemical linking via primary amines and the binding behavior of the antibodies was analyzed over 30 min. The results are shown in Table 2.

TABLE 2
B1 Binding to PDGF

| | ka | kd | Rmax | kt | RI |
|---|---|---|---|---|---|
| | (1/Ms) | (1/s) | (RU) | (RU/(M*s)) | (RU) |
| | 1.91e4 | 2.05e − 4 | 118 | 1.43e7 | |
| B1E1C1bind Fc = 2 − 2 | | | | | −2.77 |
| B1E1C1bind Fc = 2 − 3 | | | | | −1.93 |
| B1E1C1bind Fc = 2 − 4 | | | | | −2.07 |
| B1E1C1bind Fc = 2 − 5 | | | | | 1.45 |
| B1E1C1bind Ec = 2 − 6 | | | | | 3.66 |
| B1E1C1bind Fc = 2 − 7 | | | | | 5.26 |

| | Conc of | KA | KD | Req | kobs | |
|---|---|---|---|---|---|---|
| | analyte | (1/M) | (M) | (RU) | (1/s) | Chi2 |
| | | 9.29e7 | 1.08e − 8 | | | 0.784 |
| B1E1C1bind Fc = 2 − 2 | .06125 u | | | 100 | 1.37e − 3 | |
| B1E1C1bind Fc = 2 − 3 | .125 u | | | 108 | 2.59e − 3 | |
| B1E1C1bind Fc = 2 − 4 | .25 u | | | 113 | 4.97e − 3 | |
| B1E1C1bind Fc = 2 − 5 | .5 u | | | 115 | 9.74e − 3 | |
| B1E1C1bind Fc = 2 − 6 | 1 u | | | 116 | 0.0193 | |
| B1E1C1bind Fc = 2 − 7 | 2 u | | | 117 | 0.0384 | |

C1 Binding to PDGF

| | ka | kd | Rmax | kt | RI |
|---|---|---|---|---|---|
| | (1/Ms) | (1/s) | (RU) | (RU/(M*s)) | (RU) |
| | 1.07e5 | 4.62e − 5 | 153 | 5.69e7 | |
| B1E1C1bind Fc = 2 − 16 | | | | | −0.172 |
| B1E1C1bind Fc = 2 − 17 | | | | | −0.559 |
| B1E1C1bind Fc = 2 − 18 | | | | | −0.284 |
| B1E1C1bind Fc = 2 − 19 | | | | | −0.524 |
| B1E1C1bind Fc = 2 − 20 | | | | | 4.76 |
| B1E1C1bind Fc = 2 − 21 | | | | | 4.69 |

| | Conc of | KA | KD | Req | kobs | |
|---|---|---|---|---|---|---|
| | analyte | (1/M) | (M) | (RU) | (1/s) | Chi2 |
| | | 2.31e9 | 4.33e − 10 | | | 0.456 |
| B1E1C1bind Fc = 2 − 16 | 15.6 n | | | 149 | 1.71e − 3 | |
| B1E1C1bind Fc = 2 − 17 | 31.25 n | | | 151 | 3.38e − 3 | |
| B1E1C1bind Fc = 2 − 18 | 62.5 n | | | 152 | 6.71e − 3 | |
| B1E1C1bind Fc = 2 − 19 | 125 n | | | 153 | 0.0134 | |
| B1E1C1bind Fc = 2 − 20 | 250 n | | | 153 | 0.0267 | |
| B1E1C1bind Fc = 2 − 21 | 500 n | | | 153 | 0.0534 | |

E1 Binding to PDGF

| | ka | kd | Rmax | kt | RI |
|---|---|---|---|---|---|
| | (1/Ms) | (1/s) | (RU) | (RU/(M*s)) | (RU) |
| | 2.57e4 | 1.17e − 4 | 159 | 5.75e7 | |
| B1E1C1bind Fc = 2 − 9 | | | | | 0.897 |
| B1E1C1bind Fc = 2 − 10 | | | | | 1.16 |
| B1E1C1bind Fc = 2 − 11 | | | | | 3.72 |
| B1E1C1bind Fc = 2 − 12 | | | | | 3.05 |
| B1E1C1bind Fc = 2 − 13 | | | | | 7.02 |
| B1E1C1bind Fc = 2 − 14 | | | | | 2.56 |

| | Conc of | KA | KD | Req | kobs | |
|---|---|---|---|---|---|---|
| | analyte | (1/M) | (M) | (RU) | (1/s) | Chi2 |
| | | 2.19e8 | 4.56e − 9 | | | 0.73 |
| B1E1C1bind Fc = 2 − 9 | .06125 u | | | 148 | 1.69e − 3 | |
| B1E1C1bind Fc = 2 − 10 | .125 u | | | 154 | 3.33e − 3 | |
| B1E1C1bind Fc = 2 − 11 | .25 u | | | 157 | 6.53e − 3 | |
| B1E1C1bind Fc = 2 − 12 | .5 u | | | 158 | 0.013 | |
| B1E1C1bind Fc = 2 − 13 | 1 u | | | 159 | 0.0258 | |
| B1E1C1bind Fc = 2 − 14 | 2 u | | | 159 | 0.0515 | |

### EXAMPLE 8

### Inhibition of Cell Proliferation

Cell Lines

All cell lines were purchased from the American Tissue Culture Collection. They were maintained in EMEM (GibcoBRL, NY) containing 10% heat-inactivated FBS, 5 mM glutamine, 100 U/ml penicillin and 100 μg/ml streptomycin.

Cell Proliferation Assay

Cells were seeded at 2000-8000 cells/well in 96 well plates. After growing overnight in EMEM supplemented with 1% fetal bovine serum (FBS) and 5 mM glutamine, the medium was replaced and antibody was added at various concentrations in EMEM/1% FBS. Endotoxin was removed from the antibody preparations using Affi-Prep Polymyxin matrix (Bio-Rad). 3 days later, supernatants were removed and the cells were lysed with 1.5% Triton X-100. Lactate dehydrogenase (LDH) as a measure of cell number was detected in the lysates using the CytoTox 96anon-radioactive cytotoxicity assay (Promega, WI) according to the manufacturer's instructions. Values derived from samples without antibody addition were used to determine 0% inhibition and values from samples with staurosporine as positive control were used to determine 100% inhibition. The percentage of inhibition of proliferation by the antibodies was calculated based on linear regression.

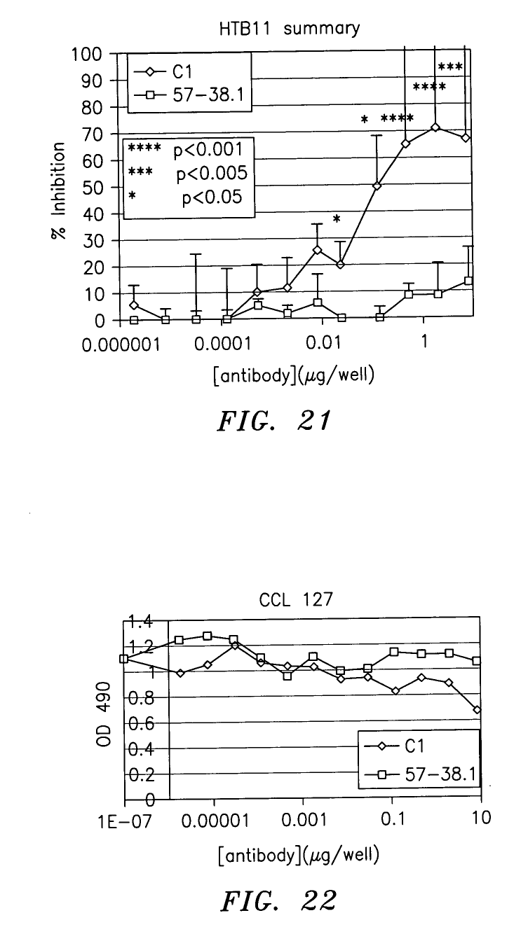

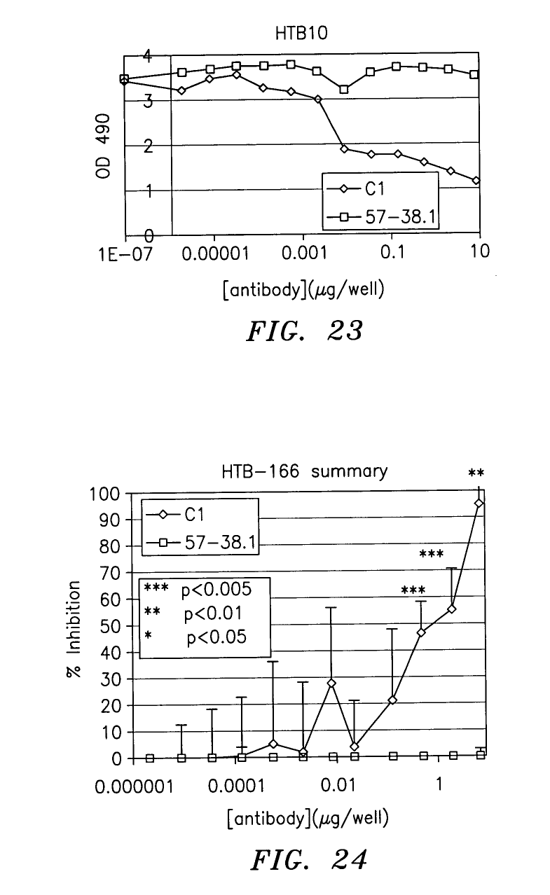

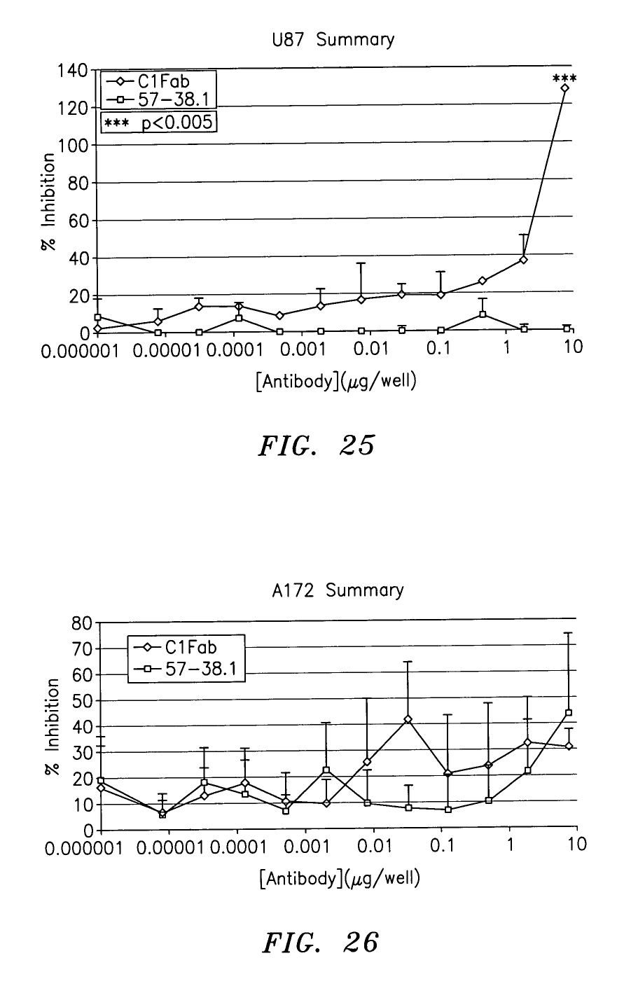

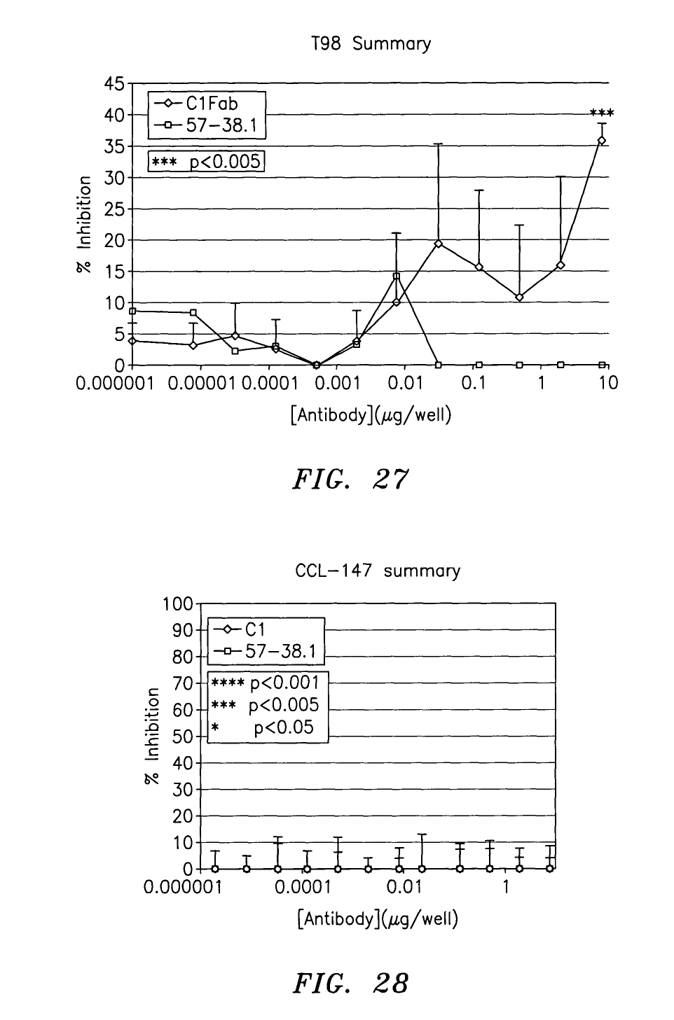

The results of the cellular proliferation assays are shown in FIGS. 21 to 28 and Table 3. Six out of 7 brain cancer cell types tested showed inhibition of proliferation when treated with anti-PDGF antibodies, but not when treated with a control antibody.

TABLE 3
Inhibition of human brain tumor cell
proliferation by anti-PDGF antibody (mouse C1)

| | | Minimum | |
|---|---|---|---|
| | | effective | |
| | % max | concentration | |
| | inhibition | (p < 0.01) | |
| HTB 11, Neuroblastoma, mets to bone | 71 | 21 | ng/well |
| CCL127, Neuroblastoma | 51 | 100 | ng/well |
| HTB 10, Neuroepithelioma, metastasis | 66 | 7.8 | ng/well |
| HTB 166, Ewing's sarcoma | 97 | 500 | ng/well |
| U87, Astrocytoma grade III | 100 | 8 | mg/well |
| A172, Glioblastoma | 0 | N/A | |
| T98 G, Glioblastoma | 36 | 8 | mg/well |

### EXAMPLE 9

### Proliferating Cell Nuclear Antigen (PCNA) Assay

Cells were seeded at 10,000 cells/well in LabTec 16-well chamber slides in EMEM supplemented with 1% FBS. The next day, medium was removed and antibody was added at various concentrations in EMEM/1 % FBS. 3 days later, the slides were washed in PBS and fixed in 70% ethanol for 20 min at 4° C. Endogenous peroxidase activity was blocked with 3% NaN3 in methanol for 10 min. PCNA expression as a marker for cell proliferation was determined using Zymed PCNA staining kit (Zymed, CA) according to the manufacturer's instruction. 5 different areas of defined size were counted in each well and the numbers were averaged. Values derived from samples without antibody addition were used to determine 0% inhibition and values from samples with staurosporine as positive control were used to determine 100% inhibition. The percentage of inhibition of proliferation by the antibodies was calculated based on linear regression.

The results are shown in Tables 4, 5 and 6.

TABLE 4
PCNA assay HTB10 cells

| | Count | Count | Count | Count | Count | | | % |
|---|---|---|---|---|---|---|---|---|
| | 1 | 2 | 3 | 4 | 5 | average | S.D. | inhibition |
| C1, 5 mg | 4 | 5 | 7 | 10 | 9 | 7 | 2.3 | 97.5 |
| C1, 5 mg | 7 | 9 | 11 | 10 | 9 | 9.2 | 1.3 | 95.5 |
| | 29 | 31 | 22 | 19 | 21 | 24.4 | 4.7 | 81.6 |
| | 21 | 39 | 18 | 20 | 17 | 23 | 8.1 | 82.9 |
| | 98 | 87 | 91 | 95 | 101 | 94.4 | 5.0 | 17.4 |
| | 92 | 89 | 91 | 97 | 92 | 92.2 | 2.6 | 19.4 |
| Control ab, 0.5 | 102 | 95 | 97 | 92 | 92 | 95.6 | 3.7 | 16.3 |
| mg | | | | | | | | |
| Control ab, 0.5 | 121 | 101 | 102 | 111 | 105 | 108 | 7.4 | 4.9 |
| mg | | | | | | | | |
| medium | 111 | 105 | 121 | 119 | 116 | 114.4 | 5.8 | 0 |
| medium | 101 | 131 | 115 | 122 | 127 | 119.2 | 10.6 | 0 |
| medium | 121 | 101 | 99 | 111 | 106 | 107.6 | 7.9 | 0 |
| medium | 105 | 111 | 102 | 123 | 121 | 112.4 | 8.4 | 0 |
| staurosporine | 5 | 2 | 3 | 4 | 3 | 3.4 | 1.0 | 100 |
| staurosporine | 7 | 2 | 5 | 5 | 4 | 4.6 | 1.6 | 100 |
| staurosporine | 5 | 6 | 2 | 4 | 4 | 4.2 | 1.3 | 100 |
| staurosporine | 7 | 5 | 6 | 3 | 4 | 5 | 1.4 | 100 |

TABLE 5
PCNA assay HTB10 cells

| | Count | Count | Count | Count | Count | | | % |
|---|---|---|---|---|---|---|---|---|
| | 1 | 2 | 3 | 4 | 5 | average | S.D. | inhibition |
| C1, 5 mg | 22 | 9 | 10 | 13 | 9 | 12.6 | 4.9 | 98.9 |
| C1, 5 mg | 11 | 8 | 7 | 14 | 16 | 11.2 | 3.4 | 99.9 |
| | 102 | 42 | 111 | 120 | 101 | 95.2 | 27.5 | 39.7 |
| | 82 | 101 | 96 | 112 | 102 | 98.6 | 9.8 | 37.3 |
| | 124 | 135 | 141 | 126 | 131 | 131.4 | 6.2 | 13.8 |
| | 150 | 61 | 142 | 139 | 156 | 129.6 | 34.8 | 15.1 |
| Control ab, 0.5 | 161 | 149 | 137 | 152 | 128 | 145.4 | 11.6 | 3.8 |
| mg | | | | | | | | |
| Control ab, 0.5 | 141 | 152 | 147 | 139 | 158 | 147.4 | 7.0 | 2.4 |
| mg | | | | | | | | |
| medium | 168 | 139 | 141 | 143 | 145 | 147.2 | 10.6 | 0 |
| medium | 139 | 157 | 154 | 138 | 142 | 146 | 7.9 | 0 |
| medium | 151 | 148 | 161 | 158 | 153 | 154.2 | 4.7 | 0 |
| medium | 155 | 171 | 139 | 152 | 161 | 155.6 | 10.5 | 0 |
| staurosporine | 5 | 11 | 4 | 10 | 7 | 7.4 | 2.7 | 100 |
| staurosporine | 17 | 11 | 13 | 11 | 11 | 12.6 | 2.3 | 100 |
| staurosporine | 8 | 16 | 12 | 11 | 12 | 11.8 | 2.6 | 100 |
| staurosporine | 15 | 9 | 17 | 10 | 10 | 12.2 | 3.2 | 100 |

TABLE 6
PCNA assay HTB166 cells

| | Count | Count | Count | Count | Count | | | % |
|---|---|---|---|---|---|---|---|---|
| | 1 | 2 | 3 | 4 | 5 | average | S.D. | inhibition |
| C1, 5 mg | 13 | 8 | 1 | 3 | 3 | 5.6 | 4.4 | 98.9 |
| C1, 5 mg | 1 | 5 | 3 | 4 | 2 | 3 | 1.4 | 100.2 |
| C1, 0.5 mg | 12 | 6 | 5 | 7 | 6 | 7.2 | 2.5 | 98.2 |
| C1, 0.5 mg | 4 | 10 | 7 | 8 | 5 | 6.8 | 2.1 | 98.4 |
| Control ab, 5 | 201 | 198 | 192 | 221 | 225 | 207.4 | 13.1 | 2.8 |
| mg | | | | | | | | |
| Control ab, 5 | 199 | 220 | 199 | 215 | 201 | 206.8 | 8.9 | 3.1 |
| mg | | | | | | | | |
| Control ab, 0.5 | 211 | 226 | 198 | 211 | 202 | 209.6 | 9.6 | 1.8 |
| mg | | | | | | | | |
| Control ab, 0.5 | 191 | 229 | 195 | 222 | 211 | 209.6 | 14.8 | 1.8 |
| mg | | | | | | | | |
| medium | 240 | 201 | 199 | 222 | 212 | 214.8 | 15.1 | 0 |
| medium | 233 | 211 | 221 | 205 | 221 | 218.2 | 9.6 | 0 |
| medium | 199 | 212 | 201 | 236 | 201 | 209.8 | 13.9 | 0 |
| medium | 197 | 205 | 213 | 222 | 215 | 210.4 | 8.6 | 0 |
| staurosporine | 5 | 2 | 0 | 2 | 3 | 2.4 | 1.6 | 100 |
| staurosporine | 6 | 1 | 3 | 3 | 4 | 3.4 | 1.6 | 100 |
| staurosporine | 7 | 5 | 4 | 2 | 3 | 4.2 | 1.7 | 100 |
| staurosporine | 6 | 2 | 3 | 4 | 2 | 3.4 | 1.5 | 100 |

### EXAMPLE 10

³H-thymidine Proliferation Assay

Cells were plated at 20,000-40,000/ml in 96 well plates in the appropriate medium containing 10% FBS. After 4 h of attachment, medium was exchanged to FBS-free medium and incubated overnight. The next day, fresh medium containing 1% FBS was added with or without anti-PDGF or control antibody. After 48 h, 1 μCi ³H-thymidine was added per well and the cells were harvested the following day using a cell harvester (Packard Instruments). Incorporated tritiated thymidine was assessed using a Topcount (Packard Instruments). Half-maximal inhibition was calculated based on linear regression. The results are shown in FIG. 10.

### EXAMPLE 11

Apoptosis Assay

To determine whether treatment of cells with the anti-PDGF antibody induces cell death, apoptosis assays were used. Apoptosis was measured using the “Cell death detection ELISA” kit from Roche (Roche Diagnostics, Germany). This assay determines cytoplasmic histone-associated DNA fragments that are only present after the induction of apoptosis. Cells were plated at 20,000 cells/ml in 96 well plates in the appropriate medium containing 10% FBS. After 4 h of attachment, medium was exchanged to FBS-free medium and incubated overnight. The next day, fresh medium containing 1% FBS was added with or without anti-PDGF or control antibody. Apoptosis was determined according to the manufacturer's instructions at various time-points and results for 2 cell lines are shown in FIG. 12. Increase in OD indicates apoptosis.

To test whether anti-PDGF induces apoptosis in a caspase-dependent way, the pan-caspase inhibitor z-vad was added. Results were compared to camptothecin-induced apoptosis and are shown in FIG. 13. As shown therein, the presence of v-zad did not greatly effect the degree of apoptosis achieved by the present antibodies. Thus, the present anti-PDGF antibodies can cause apoptosis in a largely caspase-independent manner that can be particularly useful in combination with chemotherapeutic agents conventionally used in treating various cancers.

All publications mentioned in the above specification are herein incorporated by reference. Various modifications and variations of the described embodiments, aspects and examples will be apparent to those skilled in the art without departing from the scope and spirit of the disclosure herein. Although described in connection with specific preferred embodiments, it should be understood that the subject matter should not be unduly limited to such specific embodiments. Indeed, those skilled in the art will envision various modifications which, although not specifically stated, are encompassed by the present disclosure.

What is claimed is:

1. A method for providing an engineered antibody or antibody fragment comprising:
providing a panel of antibodies having specificity for a target, the panel of antibodies including a plurality of antibody members derived from a first species;
determining the sequence of at least a portion of a first member of the panel of antibodies;
comparing sequence of said first member of the panel to a reference library of antibody sequences or antibody fragment sequences from a target species, the target species being different from the first species;
selecting at least one sequence from a first member of the reference library which demonstrates a high degree of homology to the sequence of said first member of the panel and which contains a FR3 region;
obtaining the CDR3 region from said first member of the panel; and
incorporating the CDR3 region from said first member of the panel adjacent to the FR3 region of the at least one sequence from the first member of the reference library to form an engineered antibody or antibody fragment.

2. A method for providing an engineered antibody or antibody fragment according to claim 1 further comprising the steps of:
comparing the sequence of said first member of the panel to a reference library of antibody sequences or antibody fragment sequences;
selecting at least a FR4 sequence from a second member of the reference library; and
incorporating the FR4 sequence into the engineered antibody or antibody fragment adjacent to the CDR3 region from said first member of the panel.

3. A method for providing an engineered antibody or antibody fragment according to claim 2 further comprising the step of incorporating a CDR1 region from a member of the panel into the position previously occupied by the CDR1 region of the at least one sequence from the first member of the library.

4. A method for providing an engineered antibody or antibody fragment according to claim 3 wherein the CDR3 region and the CDR1 region incorporated into the at least one sequence from the first member of the library are derived from different members of the panel.

5. A method for providing an engineered antibody or antibody fragment according to claim 3 wherein the CDR3 region and the CDR1 region incorporated into the at least one sequence from the first member of the library are derived from the same member of the panel.

6. A method for providing an engineered antibody or antibody fragment according to claim 1 further comprising the step of incorporating a CDR1 region from a second member of the panel into the position previously occupied by the CDR1 region of the at least one sequence from the first member of the library.

7. A method for providing an engineered antibody or antibody fragment according to claim 2 further comprising the step of incorporating a CDR2 region from one or more of the members of the panel into the position previously occupied by the CDR2 region of the at least one sequence from the first member of the library.

8. A method for providing an engineered antibody or antibody fragment according to claim 7 wherein the CDR3 region and the CDR2 region incorporated into the at least one sequence from the first member of the library are derived from different members of the panel.

9. A method for providing an engineered antibody or antibody fragment according to claim 7 wherein the CDR3 region and the CDR2 region incorporated into the at least one sequence from the first member of the library are derived from the same member of the panel.

10. A method for providing an engineered antibody or antibody fragment according to claim 1 further comprising the step of incorporating a CDR2 region from a second member of the panel into the position previously occupied by the CDR2 region of the at least one sequence from the first member of the library.

11. A method for providing an engineered antibody or antibody fragment according to claim 2 further comprising the step of incorporating a CDR1 and CDR2 regions from one or more of the members of the panel into the position previously occupied by the CDR1 and CDR2 regions, respectively of the at least one sequence from the first member of the library.

12. A method for providing an engineered antibody or antibody fragment according to claim 11 wherein the CDR3 region and the CDR1 region incorporated into the at least one sequence from the first member of the library are derived from different members of the panel.

13. A method for providing an engineered antibody or antibody fragment according to claim 11 wherein the CDR3 region and the CDR1 region incorporated into the at least one sequence from the first member of the library are derived from the same member of the panel.

14. A method for providing an engineered antibody or antibody fragment according to claim 11 wherein the CDR3 region and the CDR2 region incorporated into the at least one sequence from the first member of the library are derived from different members of the panel.

15. A method for providing an engineered antibody or antibody fragment according to claim 11 wherein the CDR3 region and the CDR2 region incorporated into the at least one sequence from the first member of the library are derived from the same member of the panel.

16. A method for providing an engineered antibody or antibody fragment according to claim 11 wherein the CDR3 region, the CDR2 region and the CDR1 region incorporated into the at least one sequence from the first member of the library are each derived from different members of the panel.

17. A method for providing an engineered antibody or antibody fragment according to claim 11 wherein the CDR3 region, the CDR2 region and the CDR1 region incorporated into the at least one sequence from the first member of the library are all derived from the same member of the panel.

18. A method for providing an engineered antibody or antibody fragment according to claim 1 further comprising the step of incorporating CDR1 and CDR2 regions from one or more members of the panel into the positions previously occupied by the CDR1 and CDR2 regions respectively, of the at least one sequence from the first member of the library, at least one of the CDR1 and CDR2 regions incorporated being from a second member of the panel.

19. A method for providing an engineered antibody or antibody fragment according to claim 1 wherein the target is PDGF.

20. A method for providing an engineered antibody or antibody fragment according to claim 1 wherein the antibody fragment is selected from the group consisting of scFv, Fab, F(ab′)2, Fd, diabodies, antibody light chains and antibody heavy chains.

21. A method for providing an engineered antibody or antibody fragment according to claim 1 wherein the target species is human.

22. A method for providing an engineered antibody or antibody fragment according to claim 1 wherein the reference library contains human rearranged antibody sequences.

23. A method for providing an engineered antibody library comprising the steps of preparing a plurality of engineered antibodies or antibody fragments according to the method of claim 1.

24. A method for providing an antibody library according to claim 23 wherein the library further comprises variants of the engineered antibodies or antibody fragments which include a combination of amino acids in the VHNL interface and or Vernier zone that are derived from one or more sequences selected from the group consisting of the sequences of the members of the panel, the sequence of the first member of the reference library.

25. A method for providing an antibody library according to claim 23 further comprising generating a phagemid or phage library displaying the engineered antibodies or antibody fragments.

26. A method for providing an antibody library according to claim 23 further comprising the step of panning the phagemid or phage library for activity against the target and isolating the phage or phagemid particles which preferentially bind to the target.

27. A method for choosing an antibody or antibody fragment comprising the steps of:
preparing a plurality of engineered antibodies in accordance with a method as in claim 1;
determining the binding affinity of a plurality of antibodies to a target; and
selecting an antibody from the library based on binding affinity.

28. A method for choosing an antibody or antibody fragment as in claim 27 further comprising the steps of:
determining the immunogenicity of the plurality of antibodies; and
selecting an antibody based on binding affinity and immunogenicity.

29. A method for providing an engineered antibody or antibody fragment comprising:
providing a panel of antibodies having specificity for a target, the panel of antibodies including a plurality of antibody members derived from a first species;
selecting a first member of the panel having desired binding properties with respect to said target;
determining the sequence of at least a portion of a plurality of members of the panel of antibodies to determine a consensus sequence for the plurality of members;
comparing the consensus sequence to a reference library of antibody sequences or antibody fragment sequences from a target species, the target species being different from the first species;
selecting at least one sequence from a first member of the reference library which demonstrates a high degree of homology to the consensus sequence and which contains a FR3 region;
obtaining the CDR3 region from said first member of the panel; and
incorporating the CDR3 region from the first member of the panel adjacent to the FR3 region of the at least one sequence from the first member of the library to form an engineered antibody or antibody fragment.

30. A method for providing an engineered antibody or antibody fragment according to claim 29 further comprising the steps of:
comparing the consensus sequence to a reference library of antibody sequences or antibody fragment sequences;
selecting at least a FR4 sequence from a second member of the reference library; and
incorporating the FR4 sequence into the engineered antibody or antibody fragment adjacent to the CDR3 region from said first member of the panel.

31. A method for providing an engineered antibody or antibody fragment according to claim 30 further comprising the step of incorporating a CDR1 region from a member of the panel into the position previously occupied by the CDR1 region of the at least one sequence from the first member of the library.

32. A method for providing an engineered antibody or antibody fragment according to claim 31 wherein the CDR3 region and the CDR1 region incorporated into the at least one sequence from the first member of the library are derived from different members of the panel.

33. A method for providing an engineered antibody or antibody fragment according to claim 31 wherein the CDR3 region and the CDR1 region incorporated into the at least one sequence from the first member of the library are derived from the same member of the panel.

34. A method for providing an engineered antibody or antibody fragment according to claim 29 further comprising the step of incorporating a CDR1 region from a second member of the panel into the position previously occupied by the CDR1 region of the at least one sequence from the first member of the library.

35. A method for providing an engineered antibody or antibody fragment according to claim 30 further comprising the step of incorporating a CDR2 region from one or more of the members of the panel into the position previously occupied by the CDR2 region of the at least one sequence from the first member of the library.

36. A method for providing an engineered antibody or antibody fragment according to claim 35 wherein the CDR3 region and the CDR2 region incorporated into the at least one sequence from the first member of the library are derived from different members of the panel.

37. A method for providing an engineered antibody or antibody fragment according to claim 35 wherein the CDR3 region and the CDR2 region incorporated into the at least one sequence from the first member of the library are derived from the same member of the panel.

38. A method for providing an engineered antibody or antibody fragment according to claim 29 further comprising the step of incorporating a CDR2 region from a second member of the panel into the position previously occupied by the CDR2 region of the at least one sequence from the first member of the library.

39. A method for providing an engineered antibody or antibody fragment according to claim 30 further comprising the step of incorporating a CDR1 and CDR2 regions from one or more of the members of the panel into the position previously occupied by the CDR1 and CDR2 regions, respectively of the at least one sequence from the first member of the library.

40. A method for providing an engineered antibody or antibody fragment according to claim 29 further comprising the step of incorporating CDR1 and CDR2 regions from one or more members of the panel into the positions previously occupied by the CDR1 and CDR2 regions respectively, of the at least one sequence from the first member of the library, at least one of the CDR1 and CDR2 regions incorporated being from a second member of the panel.

41. A method for providing an engineered antibody or antibody fragment according to claim 29 wherein the target is PDGF.

42. A method for providing an engineered antibody or antibody fragment according to claim 29 wherein the antibody fragment is selected from the group consisting of scFv, Fab, F(ab′)2, Fd, diabodies, antibody light chains and antibody heavy chains.

43. A method for providing an engineered antibody or antibody fragment according to claim 29 wherein the target species is human.

44. A method for providing an engineered antibody or antibody fragment according to claim 29 wherein the reference library contains human rearranged antibody sequences.

45. A method for providing an engineered antibody library comprising the steps of preparing a plurality of engineered antibodies or antibody fragments according to the method of claim 29.

46. A method for providing an antibody library according to claim 45 wherein the library further comprises variants of the engineered antibodies or antibody fragments which include a combination of amino acids in the VHNL interface and or Vernier zone that are derived from one or more sequences selected from the group consisting of the sequences of the members of the panel, the sequence of the first member of the reference library.

47. A method for providing an antibody library according to claim 45 further comprising generating a phagemid or phage library displaying the engineered antibodies or antibody fragments.

48. A method for providing an antibody library according to claim 45 further comprising the step of panning the phagemid or phage library for activity against the target and isolating the phage or phagemid particles which preferentially bind to the target.

49. A method for choosing an antibody or antibody fragment comprising the steps of:
preparing a plurality of engineered antibodies in accordance with a method as in claim 29;
determining the binding affinity of a plurality of antibodies; and
selecting an antibody based on binding affinity.

50. A method for choosing an antibody or antibody fragment as in claim 49 further comprising the steps of:
determining the immunogenicity of the plurality of antibodies; and
selecting an antibody based on binding affinity and immunogenicity.

51. A reference library of engineered antibodies or antibody fragments for selecting antibodies or antibody fragments, the library including variants of engineered antibodies or fragments, the variants having framework regions derived from an antibody native to a target species, the framework regions exhibiting a high degree of homology to the framework region of a first antibody having specificity for a predetermined target, a CDR3 region derived from the first antibody having specificity for a predetermined target, a combination of CDR1 and CDR2 regions from either the first antibody or the antibody native to the target species, and a combination of amino acids in the VHNL interface and/or Vernier zone, the combination of amino acids representing those present in the antibody native to the target species and those present in the first antibody.

52. A humanized composite antibody or functional fragment of a humanized composite antibody comprising framework regions from one or more human antibody sequences and CDR regions from two different sources, at least one of which is non-human.

53. A humanized composite antibody or functional fragment of a humanized composite antibody comprising framework regions from both germline and re-arranged human antibody sequences and CDR regions from two different sources, at least one of which is non-human.

54. A humanized composite antibody or functional fragment of a humanized composite antibody comprising framework regions from one or more human antibody sequences, a non-human CDR3 and at least one of CDR1 or CDR2 being derived from a consensus of non-human antibody sequences.

55. A method of providing a humanized composite antibody or functional fragment of a humanized composite antibody comprising combining framework regions from one or more human antibody sequences with CDR regions from two different sources, at least one of which is non-human, the framework regions being derived from human antibody sequences selected by:
(i) establishing an antibody consensus sequence for a plurality of members from a panel of non-human antibodies that bind to a target;
(ii) substituting a CDR3 from an individual member of the panel of non-human antibodies for the CDR3 of the consensus sequence to form a composite sequence; and
(iii) comparing the composite sequence to a database of human antibody sequences and selecting at least one human antibody sequence based on homology to the composite sequence.

56. A method of providing a humanized composite antibody or functional fragment of a humanized composite antibody comprising combining framework regions from two different human antibody sequences with CDR regions from two different sources, at least one of which is non-human, the framework regions being derived from two human antibody sequences selected by:
(i) establishing an antibody consensus sequence for a plurality of members from a panel of non-human antibodies that bind to a target;
(ii) substituting a CDR3 from an individual member of the panel of non-human antibodies for the CDR3 of the consensus sequence to form a composite sequence;
(iii) comparing a first portion of the composite sequence to a database of human germline antibody sequences and selecting at least one human antibody sequence based on homology to the first portion of the composite sequence; and
(iv) comparing a second portion of the composite sequence to a database of human re-arranged antibody sequences and selecting at least one human antibody sequence based on homology to the second portion of the composite sequence.

57. An engineered composite antibody sequence comprising CDR3 from an individual member of a panel of non-human antibodies that bind to a target, the balance of the engineered composite antibody sequence being derived from an antibody consensus sequence for a plurality of members from the panel of non-human antibodies.

58. In a method of preparing a humanized antibody comprising the steps of incorporating CDR regions from a non-human donor antibody into framework regions from a human acceptor antibody, the improvement comprising selecting the human acceptor antibody sequence by:
(i) establishing an antibody consensus sequence for a plurality of members from a panel of non-human antibodies that bind to a target;
(ii) substituting a CDR3 from an individual member of the panel of non-human antibodies for the CDR3 of the consensus sequence to form a composite sequence; and
(iii) comparing the composite sequence to a database of human antibody sequences and selecting at least one human acceptor antibody sequence based on homology to the composite sequence.

59. An antibody light chain comprising at least one CDR derived from a CDR selected from the group consisting of CDR1, CDR2 and CDR3 of antibody C1.

60. An antibody heavy chain comprising at least one CDR derived from a CDR selected from the group consisting of CDR1, CDR2 and CDR3 of antibody C1.

61. A method for treating a tumor comprising administering to a subject in need thereof an effective amount of an anti-PDGF antibody.

62. A method as in claim 61 wherein the tumor is selected from the group consisting of neuroblastoma, neuroepithelioma, meningiomas, Ewing's sarcoma, astrocytoma, glioblastoma, Kaposi's sarcoma, mesothelioma and mesothelioma cell lines, choriocarcinoma, pancreatic carcinoma, gastric carcinoma, osteosarcoma, esophageal cancer, fibrosarcoma, malignant epithelial cells in primary human lung carcinoma, leiomyosarcoma, liposarcoma, paraganglioma, angiosarcoma, hemangiopericytoma, sarcoma NOS, synovial sarcoma, chondrosarcoma, and uterine stromal sarcoma, mammary carcinoma, colorectal cancer, small-cell lung carcinoma, non-small cell lung cancer, malignant fibrous histiocytoma, smooth muscle cell tumor and prostrate cancer.

63. A method as in claim 61 wherein the anti-PDGF antibody is an anti-PDGF BB antibody.

64. A method as in claim 61 wherein the anti-PDGF antibody is used in combination therapy with chemotherapeutic agents.


SEQUENCE LISTING (79 sequences)

SEQ ID NO: 1 - DNA, 21 bp, artificial sequence
  linker sequence
ggtggttcctctagatcttcc

SEQ ID NO: 2 - DNA, 24 bp, artificial sequence
  primer
cggactgtggctgcaccatctgtc

SEQ ID NO: 3 - DNA, 21 bp, artificial sequence
  primer
ggccatggctggttgggcagc

SEQ ID NO: 4 - DNA, 24 bp, artificial sequence
  primer
gcctccaccaagggcccatcggtc

SEQ ID NO: 5 - DNA, 24 bp, artificial sequence
  primer
agcgtagtccggaacgtcgtacgg

SEQ ID NO: 6 - DNA, 23 bp, artificial sequence
  primer
gcggggcccaggcggccgagctc

SEQ ID NO: 7 - DNA, 21 bp, artificial sequence
  primer
gctgcccaaccagccatggcc

SEQ ID NO: 8 - DNA, 43 bp, artificial sequence
  Primer
gaggaggaggaggagtctagaattaacactcattcctgttgaa

SEQ ID NO: 9 - DNA, 39 bp, artificial sequence
  primer
gaggaggaggaggagactagtacaactcctgggcacaat

SEQ ID NO: 10 - DNA, 264 bp, NZW rabbit
gagctcgatctgacccagactccagcctccgtgtctgaacctgtgggaggcacagtcaccatcaattgccaggccagtcagagcattagtagctacttagcctggtatcagcagaaaccagggcagcctcccaagctcctgatctatgatgcatccgatctggcatctggggtcccatcgcggttcaaaggcagtggatctgggacagagtacactctcaccatcagcgacctggagtctcccgatgctgccacttactactgt

SEQ ID NO: 11 - DNA, 264 bp, human
gacatccagatgacccagtctccatcctccctgtctgcatctgtaggagacagagtcaccatcacttgccgggcgagtcagggcattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctcaagatgttgcaacttattactgt

SEQ ID NO: 12 - DNA, 264 bp, human
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctatgatgcctccagtttggaaagtggggtcccatcaaggttcagcggcagtggatctgggacagaattcactctcaccatcagcagcctgcagcctgatgattttgcaacttattactgc

SEQ ID NO: 13 - DNA, 264 bp, human
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgt

SEQ ID NO: 14 - DNA, 264 bp, human; identical to SEQ ID NO: 13

SEQ ID NO: 15 - DNA, 264 bp, human
gccatccagttgacccagtctccatcctccctgtctgcatctgtaggagacagagtcaccatcacttgccgggcaagtcagcgcattagcagtgctttagcctgatatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgt

SEQ ID NO: 16 - DNA, 288 bp, NZW rabbit
cagtcggtggaggagtccaggggaggtctcctcaagccaacggaaaccctgacactcacctgcacagtctctggattctcccccagtaaaaatgtaataagttgggtccgccaggctccagggaacgggctggaatggatcggattcattaatactggtagtagcgcatactacgcgagctgggcggaaagccgatccaccatcaccagaaacaccaacctgaacacggtggctctgaaaatgaccagtctgacagccgcggacacggccacgtatttctgtgcgaga

SEQ ID NO: 17 - DNA, 294 bp, human
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgaga

SEQ ID NO: 18 - DNA, 291 bp, human
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagttacatatactacgcagactcagtgaagggccgattcaccatctccagagacaacgccaagaactcactgtatctgcaaatgaacagcctgagagccgaggacacggctgtgtattactgtgcgaga

SEQ ID NO: 19 - DNA, 288 bp, human
gaggtgcagctggtggagtctgggggaggcttggtacagcctagggggtccctgagactctcctgtgcagcctctggattcaccgtcagtagcaatgagatgagctggatccgccaggctccagggaaggggctggagtgggtctcatccattagtggtggtagcacatactacgcagactccaggaagggcagattcaccatctccagagacaattccaagaacacgctgtatcttcaaatgaacaacctgagagctgagggcacggccgcgtattactgtgccaga

SEQ ID NO: 20 - DNA, 294 bp, human
gaggtgcaactggtggagtctgggggaggcctggtcaagcctggggggtccctgagactctcctgtccagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgaga

SEQ ID NO: 21 - DNA, 291 bp, human
gaggtgcagctggtggagtctggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgaga

SEQ ID NO: 22 - DNA, 87 bp, artificial sequence
  oligonucleotide
gaggaggaggaggagggccgagatccagatgacccagtctccatcctccctgtctgcatctgtaggagacagagtcaccatcacttg

SEQ ID NO: 23 - DNA, 95 bp, artificial sequence
  oligonucleotide
gagcttaggarstttccctggtttctgctgataccaggctaagtaattactaatgccctgactcgcccggcaagtgatggtgactctgtctccta

SEQ ID NO: 24 - DNA, 95 bp, artificial sequence
  oligonucleotide
gagcttaggarstttccctggtttctgctgataccaggctaagtagctactaatgctctgactggcctggcaagtgatggtgactctgtctccta

SEQ ID NO: 25 - DNA, 110 bp, artificial sequence
  oligonucleotide
accagggaaasytcctaagctcctgatctatgatgcatccgatctggcatctggggtcccatctcggttcartggcagtggatctgggacagattwcactctcaccatca

SEQ ID NO: 26 - DNA, 101 bp, artificial sequence
  oligonucleotide
gaaagtattatcaacattactactactataaccctgttgacagtaataagttgcaacatcttcaggctgcaggctgctgatggtgagagtgwaatctgtcc

SEQ ID NO: 27 - DNA, 100 bp, artificial sequence
  oligonucleotide
gaagtattatcaacattactactactataaccctgttgacagtaataagttgcagcatcttcacactgcaggctgctgatggtgagagtgwaatctgtcc

SEQ ID NO: 28 - DNA, 61 bp, artificial sequence
  oligonucleotide
gggttatagtagtagtaatgttgataatactttcggcggagggaccgaggtggtcgtcaaa

SEQ ID NO: 29 - DNA, 44 bp, artificial sequence
  oligonucleotide
gacagatggtgcagccacagttcgtttgacgaccacctcggtcc

SEQ ID NO: 30 - DNA, 110 bp, artificial sequence
  oligonucleotide
gctgcccaaccagccatggccgaaggtgcagctggtggagtctgggggaggcctggtcaagcctggggggtccctgagactctcctgtgcagcctctggattcwccyyca

SEQ ID NO: 31 - DNA, 69 bp, artificial sequence
  oligonucleotide
cagccccttccctggagcctggcggacccagttcatgctatagctactgrrggwgaatccagaggctgc

SEQ ID NO: 32 - DNA, 68 bp, artificial sequence
  oligonucleotide
cagccccttccctggagcctggcggacccaacttattacatttttactgrrggwgaatccagaggctg

SEQ ID NO: 33 - DNA, 111 bp, artificial sequence
  oligonucleotide
ggctccagggaaggggctggagtggatcggattcattaatactggtagtagcgcatactacgcgagctgggcggaaagccgatycaccatctccagagacamcgccaagaa

SEQ ID NO: 34 - DNA, 111 bp, artificial sequence; identical to SEQ ID NO: 33
  oligonucleotide

SEQ ID NO: 35 - DNA, 88 bp, artificial sequence
  oligonucleotide
cctctcgcacagwaatacacagccgtgtcctcggctctcaggctgttcatttgcagaaatacastgagttcttggcgktgtctctgga

SEQ ID NO: 36 - DNA, 88 bp, artificial sequence
  oligonucleotide
ggctgtgtattwctgtgcgagaggtagtcctggttacagtgatggacttaacatctggggccagggcaccctggtcaccgtctcctca

SEQ ID NO: 37 - DNA, 44 bp, artificial sequence
  oligonucleotide
gaccgatgggcccttggtggaggctgaggagacggtgaccaggg

SEQ ID NO: 38 - DNA, 1514 bp, artificial sequence
  Assembled Degenerate Oligonucleotides
gaggaggaggaggagggcccgagatccagatgacccagtctccatcctccctgtctgcatctgtaggagacagagtcaccatcacttgccrggcsagtcagrgcattagtarytacttagcctggtatcagcagaaaccagggaaasytcctaagctcctgatctatgatgcatccgatctggcatctggggtcccatctcggttcartggcagtggatctgggacagattwcactctcaccatcagcagcctgcagystgaagatgytgcaacttattactgtcaacagggttatagtagtagtaatgttgataatactttcggcggagggaccgaggtggtcgtcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagcttgcccgtcacaaagagcttcaacaggggagagtgttagttctagataattaattaggaggaatttaaaatgaaatacctattgcctacggcagccgctggattgttattactcgctgcccaaccagccatggccgaggtgcagctggtggagtctgggggaggcctggtcaagcctggggggtccctgagactctcctgtgcagcctctggattcwccyycagtarmwatrkmatrarytgggtccgccaggctccagggaaggggctggagtggrtcksattcattaatactggtagtagcgcatactacgcgagctgggcggaaagccgatycaccatctccagagacamcgccaagaactcastgtatctgcaaatgaacagcctgagagccgaggacacggctgtgtattwctgtgcgagaggtagtcctggttacagtgatggacttaacatctggggccagggcaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagtggccaggccggccagcaccatcaccatcaccatggcgcatacccgtacgacgttccggactacgct

SEQ ID NO: 39 - protein, 217 aa, artificial sequence
  Humanized Rabbit Light Chain
ELQMTQSPSSLSASVGDRVTITCXASQXISXYLAWYQQKPGKXPKLLIYDASDLASGVPSRFXGSGSGTDXTLTISSLQXEDXATYYCQQGYSSSNVDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGEC

SEQ ID NO: 40 - protein, 246 aa, artificial sequence
  Humanized Rabbit Heavy Chain
MKYLLPTAAAGLLLLAAQPAMAEVQLVESGGGLVKPGGSLRLSCAASGFXXSXXXXXWVRQAPGKGLEWXXFINTGSSAYYASWAESRXTISRDXAKNSXYLQMNSLRAEDTAVYXCARGSPGYSDGLNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 41 - protein, 110 aa, artificial sequence
  Humanized Light Chain
ELQMTQSPSSLSASVGDRVTITCRASQGISNYLAWYQQKPGKPPKLLIYDASDLASGVPSRFNGSGSGTDFTLTISSLQPEDVATYYCQQGYSSSNVDNTFGGGTEVVVK

SEQ ID NO: 42 - protein, 111 aa, artificial sequence
  Humanized Light Chain
ELQMTQSPSSLPASVGDRVTITCQASQSISNYLAWYQQKPGKAPKLLIYDASDLASGVPSRFNGSGSGTDDYTLTISSLQPEDVATYYCQQGYSSSNVDNTFGGGTEVVVK

SEQ ID NO: 43 - protein, 110 aa, artificial sequence
  Humanized Light Chain
ELQMTQSPSSLSASVGDRVTITCRASQGISNYLAWYQQKPGKAPKLLIYDASDLASGVPSRFSGSGSGTDYTLTINSLQPEDVATYYCQQGYSSSNVDNTFGGGTEVVVK

SEQ ID NO: 44 - protein, 119 aa, artificial sequence
  Humanized Heavy Chain
EVQLVESGGGLVKPGGTLRLSCAASGFTPSSYSMNWYRQAPGKGLEWVSFINTGSSAYYASWAESRFTISRDNAKNSLYLQMNSLRAEDTAVYYCARGSPGYSDGLNIWGPGTLVTVSS

SEQ ID NO: 45 - protein, 119 aa, artificial sequence
  Humanized Heavy Chain
EVQLVESGGGLVKPGGSLRLSCAASGFTPSKNVISWVRQAPGKGLEWIGFINTGSSAYYASWAESRSTISRDTAKNSLYLQMNSLRAEDTAVYYCARGSPGYSDGLNIWGPGTLVTVSS

SEQ ID NO: 46 - protein, 119 aa, artificial sequence
  Humanized Heavy Chain
EVQLVESGGGLVKPGGSLRLSCAASGFTFSKNVISWVRQAPGKGLEWIGFINTGSSAYYASWAESRFTISRDNAKNSVYLQMNSLRAEDTAVYYCARGSPGYSDGLNIWGPGTLVTVSS

SEQ ID NO: 47 - DNA, 702 bp, murine
gagctcgtccaggtgctcacccagtctccagcaatcatgtctgcatctccaggggagaaggtcaccataacctgcaactgccagctcaagtgtaagwtacatgcactggttccagcagaagccaggcacttctcccaaactcttgatttatagcrcatccaacctggcttctggagtccctgctcgcttcagtggcagtggatctgggacctcttactctctcacaatcagccgaatggaggctgaagatgctgccacttattactgccagcaaaggagtagttacccatggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaattctagataattaattaggaggaatttaaaatgaaatacctattgcgtacggc

SEQ ID NO: 48 - DNA, 741 bp, murine
caggtgctgctcgagcagtctgggkctgagctggtgaggcctggggcttcagtgaaactgtcctgcaaggcttctggctacacgttcaccagcacctggatgaactgggttaagaagaggcctgaccaaggccttgagtggattggaaggattgatccttacgatagtgaarytcactacaatcaaaagttcaaggacaaggccatattgactgtagacaaatcctccagcacagcctacatgcaactcagcaggctgacatctgaggactctgcggtctattactgtgcaagaggggggcatatgattacgcctgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtactagtggccaggccggccagcaccatcaccatcaccatggcgcatacccgtacgacgttccggactacgcttct

SEQ ID NO: 49 - protein, 107 aa, artificial sequence
  Humanized Light Chain
ELQLTQSPSSLSASVGDRVTITCTASSSVRYMHWYQQKPGKAPKLLIYSTSNLASGVPSRFSGSGSGTDYTLTISSLEPEDVATYYCQQRSKLPWTFGGGTKLEIKR

SEQ ID NO: 50 - protein, 106 aa, artificial sequence
  Humanized Light Chain
ELQLTQSPSSLSASVGDRVTITCTASSSVRYMHWYQQKPGKAPKLLIYSASNLASGVPSRFSGSGSGTDYTLTISSLEPEDVATYYCQQRSSPWTFGGGTKLEIKR

SEQ ID NO: 51 - protein, 107 aa, artificial sequence
  Synthetic Murine C1
ELQLTQSPSSLSASVGDRVTITCTASSSVXYMHWXQQKPGKKPKLLIYSXSNLASGVPSRFSGSGSGTDXTLTISSLEPEDVATYYCQQRSSYPWTFGGGTKLEIKR

SEQ ID NO: 52 - protein, 108 aa, murine
LVQVLTQSPAIMSASPGEKVTITCTASSSVSYMHWFQQKPGTSPKLLIYSASNLASGVPARFSGSGSGTSYSLTISRMEAEDAATYYCQQRSSYPWTFGGGTKLEIKR

SEQ ID NO: 53 - protein, 246 aa, artificial sequence
  Humanized Heavy Chain
ESGAEVKKPGASVKVSCKASGYTFTSTWMNWVRQTPDQGLEWIGRIDPYDSAEHYNQKFKDRVTMTVDKSISTAYMQLSRLRSEDTAVYYCARGGHMITPAMDYWGQGTLVTVSSASTKGPSVESGAEVKKPGASVKVSCKASGYTFTSTWMNWVRQAPDQGLEWIGRIDPYDSAEHYNQKFKDRVTMTVDKSISTAYMQLSRLRSEDTAVYYCARGGHMITPAMDYWGQGTLVTVSSASTKGPSV

SEQ ID NO: 54 - protein, 123 aa, artificial sequence
  Humanized Heavy Chain
ESGAEVKKPGASVKVSCKASGYTFTSTWMNWVKQTPDQGLEWIGRIDPYDSEAHYNQKFKDRVTMTVDTSISTAYMELSRLRSEDTAVYYCARGGHMITPAMDYWGQGTLVTVSSASTKGPSV

SEQ ID NO: 55 - protein, 123 aa, artificial sequence
  Humanized Heavy Chain
ESGAEVKKPGASVKVSCKASGYTFTSTWMNWVKQAPDQGLEWIGRIDPYDSEAHYNQKFKDRVTMTVDTSISTAYMELSRLRSEDTAVYYCARGGHMITPAMDYWGQGTLVTVSSASTKGPSV

SEQ ID NO: 56 - protein, 123 aa, artificial sequence
  Synthetic Murine C1
ESGAEVKKPGASVKVSCKASGYTFTSTWMNWVXXXPDQGLEWIGRIDPYDSEXHYNQKFKDXVTXTXDXSISTAYMXLSRLRSEDXAVYYCARGGHMITPAMDYWGQGTLVTVSSASTKGPSV

SEQ ID NO: 57 - protein, 114 aa, murine
QSGSELVRPGASVKLSCKASGYTFTSTWMNWVKKPDQGLEWIGRIDPYDSETHYNQKFKDKAILTVDKSSSTAYMQLSRLTSEDSAVYYCARGGHMITPAMDYWGQGTSVTVSS

SEQ ID NO: 58 - DNA, 365 bp, artificial sequence
  Assembled Oligonucleotides for Humanized Light Chain
gggcccaggcggccgagctccagttgacccagtctccatcctccctgtctgcatctgtaggagacagagtcaccatcacctgcacctgcactgccagctcaagtgtaagwtacatgcactggtwycagcagaagccaggcaaagttcccaaactcytgatttatagcrcatccaacctggcttctggagtcccttctcgcttcagtggcagtggatctgggaccgattwcactctcacaatcagcagcctggagcctgaagatgttgccacttattactgccagcaaaggagtagttacccatggacgttcggtggaggcaccaagctggaaatcaaacggcgaactgtggctgcaccatctgtc

SEQ ID NO: 59 - DNA, 394 bp, artificial sequence
  Assembled Oligonucleotides for Humanized Heavy Chain
gctgcccaaccagccatggccctgcagtctggggctgaggtgaagaagcctggggcttcagtgaaagtctcctgcaaggcttctggctacacgttcaccagcacctggatgaactgggtthrrmagrsscctgaccaaggcttgagtggattggaaggattgatccttacgatagtgaarytcactacaatcaaaagttcaaggacarggtcaccatgactrkagacamatccatcagcacagcctacatgsarctcagcaggctgagatctgaggacwckgcggtctattactgtgcaagaaggggggcatatgattacgcctgctatggactactggggtcaaggaaccctggtcaccgtctcctcagcctccaccaaggggcccatcggtc

SEQ ID NO: 60 - DNA, 80 bp, artificial sequence
  Oligonucleotide
gggcccaggcggccgagctccagttgacccagtctccatcctccctgtctgcatctgtaggagacagagtcaccatcacc

SEQ ID NO: 61 - DNA, 78 bp, artificial sequence
  Oligonucleotide
gcttctgctgrwaccagtgcatgtawcttacacttgagctggcagtgcaggtgcaggtgatggtgactctgtctccta

SEQ ID NO: 62 - DNA, 78 bp, artificial sequence
  Oligonucleotide
gcttctgctgrwaccaatttaaataactgcttatgccgctctgactgcaggtgcaggtgatggtgactctgtctccta

SEQ ID NO: 63 - DNA, 71 bp, artificial sequence
  Oligonucleotide
tgcactggtwycagcagaagccaggcaaakytcccaaactcytgatttatagcrcatccacctggcttctg

SEQ ID NO: 64 - DNA, 72 bp, artificial sequence
  Oligonucleotide
taaattggtwycagcagaagccaggcaaagttcccaaactcytgatttatagcrcatccaacctggcttctg

SEQ ID NO: 65 - DNA, 72 bp, artificial sequence
  Oligonucleotide
tgcactggtwycagcagaagccaggcaaagttcccaaactcytgatttatagcrcatccaacttgcaatctg

SEQ ID NO: 66 - DNA, 72 bp, artificial sequence
  Oligonucleotide
taaattggtwycagcagaagccaggcaaagttcccaaactcytgatttatagcrcatccaacttgcaatctg

SEQ ID NO: 67 - DNA, 73 bp, artificial sequence
  Oligonucleotide
ttgtgagagtgwaatcggtcccagatccactgccactgaagcgagaagggactccagaagcaggttggatgyg

SEQ ID NO: 68 - DNA, 74 bp, artificial sequence
  Oligonucleotide
ttgtgagagtgwaatcggtcccagatccactgccactgaagcgagaagggactccagattgcaagttggatgyg

SEQ ID NO: 69 - DNA, 76 bp, artificial sequence
  Oligonucleotide
ggaccgattwcactctcacaatcagcagcctggagcctgaagatgttgccacttattactgccagcaaaggagtag

SEQ ID NO: 70 - DNA, 96 bp, artificial sequence
  Oligonucleotide
gacagatggtgcagccacagttcgccgtttgatttccagcttggtgcctccaccgaacgtccatgggtaactactcctttgctggcagtaataagt

SEQ ID NO: 71 - DNA, 85 bp, artificial sequence
  Oligonucleotide
gctgcccaaccagccatggccctgcagtctggggctgaggtgaagaagcctggggcttcagtgaaagtctcctgcaaggcttctg

SEQ ID NO: 72 - DNA, 89 bp, artificial sequence
  Oligonucleotide
tccactcaaggccttggtcaggssyctkyydaacccagttcatccaggtgctggtgaacgtgtagccagaagccttgcaggagactttc

SEQ ID NO: 73 - DNA, 89 bp, artificial sequence
  Oligonucleotide
tccactcaaggccttggtcaggssyctkyydaacccagtgcatatagtaggcggtgaacgtgtagccagaagccttgcaggagactttc

SEQ ID NO: 74 - DNA, 85 bp, artificial sequence
  Oligonucleotide
ctgaccaaggccttgagtggattggaaggattgatccttacgatagtgaarytcactacaatcaaaagttcaaggacarggtcac

SEQ ID NO: 75 - DNA, 85 bp, artificial sequence
  Oligonucleotide
ctgaccaaggccttgagtggattggatggattaacccttacaatggtggcrytaactacgcacaaaagttcacgggcarggtcac

SEQ ID NO: 76 - DNA, 60 bp, artificial sequence
  Oligonucleotide
atgtaggctgtgctgatgrwtktgtctmyagtccatgtgaccytgtccttgaacttttga

SEQ ID NO: 77 - DNA, 60 bp, artificial sequence
  Oligonucleotide
atgtaggctgtgctgatgrwtktgtctmyagtccatgtgaccytgcccgtgaacttttga

SEQ ID NO: 78 - DNA, 94 bp, artificial sequence
  Oligonucleotide
camawycatcagcacagcctacatgsarctcagcaggctgagatctgaggacwckgcggtctattactgtgcaagaggggggcatatgattacg

SEQ ID NO: 79 - DNA, 94 bp, artificial sequence
  Oligonucleotide
gaccgatgggcccttggtggaggctgaggagacggtgaccagggttccttgaccccagtagtccatagcaggcgtaatcatatgcccccctctt